(19) United States
(12) Patent Application Publication
(10) Pub. No.: US 2026/0199249 A1
(43) Pub. Date: Jul. 16, 2026

(54) SOLID ELACESTRANT DIHYDROCHLORIDE COMPOSITIONS, METHODS OF MAKING THE SAME, AND METHODS OF TREATMENT USING THE SAME

(71) Applicant: BERLIN-CHEMIE AG, Berlin (DE)

(72) Inventor: Matthew D. BURKE, Wayne, PA (US)

(21) Appl. No.: 19/409,626

(22) Filed: Dec. 4, 2025

Related U.S. Application Data
(63) Continuation of application No. PCT/IB2024/055420, filed on Jun. 4, 2024.
(60) Provisional application No. 63/471,258, filed on Jun. 5, 2023.

(51) Int. Cl. A61K 9/20; A61K 9/28; A61K 31/136

(52) U.S. Cl. CPC A61K 9/2077; A61K 9/2009; A61K 9/2013; A61K 9/2027; A61K 9/2054; A61K 9/2095; A61K 9/284; A61K 31/136

(57) ABSTRACT

Solid pharmaceutical compositions of elacestrant dihydrochloride with high hardness values increase the dissolution time for elacestrant. Methods of making solid pharmaceutical elacestrant dihydrochloride compositions are disclosed, in which a mixture comprising elacestrant dihydrochloride and one or more pharmaceutically acceptable excipients is compressed at a pressure of at least 1 kN to produce a tablet with high hardness and a favorable dissolution profile. Methods of treatment for breast cancer include administering the solid pharmaceutical composition to a human subject.

### CROSS-REFERENCE TO RELATED APPLICATIONS

This Application is a continuation of International Application No. PCT/IB2024/055420, filed on Jun. 4, 2024, which claims the benefit of U.S. Provisional Application No. 63/471,258, filed Jun. 5, 2023, the disclosures of which are hereby incorporated by reference in their entirety.

### BACKGROUND

The following description of the background of the present technology is provided simply as an aid in understanding the present technology and is not admitted to describe or constitute prior art to the present technology.

Elacestrant dihydrochloride is a drug under development for the treatment of breast cancer. ORSERDU® (elacestrant) immediate release (IR) film-coated tablets, containing 86 mg and 345 mg elacestrant free-base, corresponding to 100 mg and 400 mg of elacestrant dihydrochloride, respectively (hereinafter, the free-base or salt strengths may be used interchangeably), were approved in 2023 by the FDA under NDA 217639 for the treatment of postmenopausal women or adult men with estrogen receptor (ER) positive, human epidermal growth factor receptor 2 (HER2)-negative, and estrogen receptor 1 (ESR1)-mutated advanced or metastatic breast cancer with disease progression following at least one line of endocrinological therapy. Thus, formulations for safe, effective delivery of elacestrant dihydrochloride are highly advantageous.

### SUMMARY

In one aspect, which may be combined with any other aspect or embodiment, the present disclosure relates to a solid pharmaceutical composition comprising: a core comprising: elacestrant dihydrochloride, present at a concentration of 30 wt. % to 60 wt. %, relative to the total weight of the composition; and one or more pharmaceutically acceptable excipients, wherein the pharmaceutical composition is a tablet having a hardness of at least 5 kP. In some embodiments, the tablet has a hardness of at least 7 kP. In some embodiments, the tablet has a hardness of at least 13 kP.

In some embodiments, the elacestrant dihydrochloride is present in an amount of 50 mg to 500 mg. In some embodiments, the elacestrant dihydrochloride is present in an amount of about 100 mg, about 172 mg, about 258 mg, or about 400 mg. In some embodiments, the elacestrant dihydrochloride is present at a concentration of 40 wt. % to 60 wt. %, relative to the total weight of the composition. In some embodiments, the elacestrant dihydrochloride is present at a concentration of 44 wt. % to 47 wt. %, relative to the total weight of the composition.

In some embodiments, at least 75% of the elacestrant dihydrochloride is dissolved at 45 minutes in water at pH 4.5 or less. In some embodiments, at least 80% of the elacestrant dihydrochloride is dissolved after 45 minutes in water at pH 4.5 or less.

In some embodiments, the pharmaceutical excipients comprise one or more of microcrystalline cellulose, crospovidone, silicified microcrystalline cellulose (SMCC), colloidal silicon dioxide, or magnesium stearate. In some embodiments, the composition further comprises a film coating the core. In some embodiments, the film is present at a concentration of about 1 wt. % to about 5 wt. %, relative to the total weight of the composition.

In some embodiments, the elacestrant dihydrochloride is present in granules. In some embodiments, the composition comprises SMCC, present at a concentration of about 5.0 wt. % to about 20.0 wt. %, relative to the total weight of the composition. In some embodiments, the composition comprises SMCC, present at a concentration of about 10.0 wt. % to about 20.0 wt. %, relative to the total weight of the composition.

In some embodiments, the composition is effective to achieve a plasma elacestrant free base Cₘᵢₙ at steady state of greater than or equal to 20 ng/mL in a human subject at 30 hours after a single administration. In some embodiments, the composition is effective to achieve a plasma elacestrant free base Cₘᵢₙ at steady state of greater than or equal to 30 ng/ml in a human subject at 30 hours after a single administration.

In another aspect, which may be combined with any other aspect or embodiment, the present disclosure relates to a method of manufacturing a solid pharmaceutical composition, the method comprising: compressing a mixture comprising elacestrant dihydrochloride and one or more pharmaceutically acceptable excipients to produce a tablet, wherein: the mixture comprises 30 wt. % to 60 wt. % of elacestrant dihydrochloride, relative to the total weight of the mixture; and the compressing is performed at a pressure of at least 1 kN; and the tablet has a hardness of at least 5 kP. In some embodiments, the method further comprises coating the tablet with a film.

In some embodiments, the compressing performed at a pressure of at least 9 kN. In some embodiments, the tablet has a hardness of at least 7 kP. In some embodiments, the tablet has a hardness of at least 13 kP.

In some embodiments, the elacestrant dihydrochloride is present in an amount of 50 mg to 500 mg in the tablet. In some embodiments, the elacestrant dihydrochloride is present in the tablet in an amount of about 100 mg, about 172 mg, about 258 mg, or about 400 mg. In some embodiments, the elacestrant dihydrochloride is present in the mixture at a concentration of 40 wt. % to 60 wt. %, relative to the total weight of the mixture. In some embodiments, the elacestrant dihydrochloride is present in the mixture at a concentration of 44 wt. % to 47 wt. %, relative to the total weight of the mixture.

In some embodiments, at least 75% of the elacestrant dihydrochloride in the tablet is dissolved at 45 minutes in water at pH 4.5 or less. In some embodiments, at least 80% of the elacestrant dihydrochloride in the tablet is dissolved after 45 minutes in water at pH 4.5 or less.

In some embodiments, the mixture further comprises one or more of microcrystalline cellulose, crospovidone, silicified microcrystalline cellulose (SMCC), colloidal silicon dioxide, or magnesium stearate.

In some embodiments, the elacestrant dihydrochloride is present in granules. In some embodiments, the mixture comprises SMCC, present at a concentration of about 5.0 wt. % to about 20.0 wt. %, relative to the total weight of the mixture. In some embodiments, the mixture comprises SMCC, present at a concentration of about 10.0 wt. % to about 20.0 wt. %, relative to the total weight of the mixture.

In some embodiments, the tablet is effective to achieve a plasma elacestrant free base Cₘᵢₙ at steady state of greater than or equal to 20 ng/mL in a human subject (e.g., at 30 hours after a single administration). In some embodiments, the tablet is effective to achieve a plasma elacestrant free base Cₘᵢₙ at steady state of greater than or equal to 30 ng/ml in a human subject (e.g., at 30 hours after a single administration).

In another aspect, which may be combined with any other aspect or embodiment, the present disclosure relates to a method for treating breast cancer in a subject, the method comprising administering to the subject an effective amount of the solid pharmaceutical composition according to any of the embodiments disclosed herein.

In some embodiments, the breast cancer is an estrogen receptor positive (ER+) breast cancer and/or a human epidermal growth factor receptor 2 (HER2)-negative (HER2−) breast cancer. In some embodiments, the breast cancer is an ER+ and estrogen receptor 1 (ESR1)-mutated breast cancer. In some embodiments, the breast cancer is ER+/HER2− and estrogen receptor 1 (ESR1)-mutated breast cancer. In some embodiments, the breast cancer is an advanced or metastatic breast cancer. In some embodiments, the ER+ breast cancer is an estrogen receptor alpha positive (ERα+) breast cancer.

In some embodiments, the breast cancer has progressed after endocrinological treatment. In some embodiments, the breast cancer has progressed following at least one line of endocrine therapy. In some embodiments, the endocrinological therapy comprises administration of one or more drugs selected from: a selective estrogen receptor degrader (SERD), an aromatase inhibitor, a selective estrogen receptor modulator (SERM), a human epidermal growth factor receptor 2 (HER2) inhibitor, a chemo therapeutic agent, a cdk4/6 inhibitor, an m-TOR inhibitor, a phosphoinositide 3-kinase inhibitors (PI3K inhibitor), or rituximab.

In some embodiments, the solid pharmaceutical composition is administered at least once per day. In some embodiments, the solid pharmaceutical composition is administered once per day. In some embodiments, the solid pharmaceutical composition is administered by oral administration. In some embodiments, the subject is a postmenopausal woman or adult man.

Additional aspects and/or embodiments of the invention will be provided, without limitation, in the detailed description of the present technology set forth below. The following detailed description is exemplary and explanatory, but it is not intended to be limiting.

### BRIEF DESCRIPTION OF THE DRAWINGS

Various objects, aspects, features, and advantages of the disclosure will become more apparent and better understood by referring to the detailed description taken in conjunction with the accompanying figures.

FIG. 1 is a flowchart that represents the process of preparing the solid pharmaceutical compositions of some embodiments.

FIG. 2 is a flowchart that represents the process of preparing the solid pharmaceutical compositions of some embodiments.

FIG. 3 is a plot of strain rate sensitivity versus yield pressure for elacestrant dihydrochloride.

FIG. 4 shows dissolution of elacestrant dihydrochloride tablets (400 mg), compressed at different hardness values.

FIG. 5 shows dissolution of elacestrant dihydrochloride tablets (400 mg) for low- and high-hardness prototypes of Formulation 2 and a Phase 3 comparator (Formulation 1).

FIG. 6 is a plot comparing the dissolution of 100 mg tablets of Formulation 2 prepared with different compression forces and having different tablet hardness.

FIG. 7 is a plot comparing the dissolution (using QC release method (0.01 N HCl)) of 100 mg tablets of Formulation 2 and the Phase 3 comparator (Formulation 1) prepared with different compression forces and having different tablet hardness.

FIG. 8 is a plot comparing the dissolution (using QC release method (0.01 N HCl)) of 400 mg tablets of Formulation 2 and the Phase 3 Comparator (Formulation 1) prepared with different compression forces and having different tablet hardness.

FIG. 9 shows dissolution data for 100 mg tablets of Formulation 2 at target hardness compared to the Phase 3 comparator (Formulation 1) in QC medium (0.01 N HCl).

FIG. 10 shows dissolution data for 400 mg tablets of Formulation 2 at target hardness compared to the Phase 3 comparator (Formulation 1) in QC medium (0.01 N HCl).

FIGS. 11A-D show a dissolution profile comparison of 100-mg dosage strength Formulation 2 at pH 1.2 (FIG. 11A), 2.0 (QC) (FIG. 11B), 4.5 (FIG. 11C), and 6.8 (FIG. 11D).

FIGS. 12A-D show a dissolution profile comparison of 400-mg dosage strength Formulation 2 at pH 1.2 (FIG. 12A), 2.0 (QC) (FIG. 12B), 4.5 (FIG. 12C), and 6.8 (FIG. 12D).

FIGS. 13A-D show a dissolution profile comparison of 100-mg dosage strength Formulation 2 and the Phase 3 comparator (Formulation 1) at pH 1.2 (FIG. 13A), 2.0 (QC) (FIG. 13B), 4.5 (FIG. 13C), and 6.8 (FIG. 13D).

FIGS. 14A-D show a dissolution profile comparison of 400-mg dosage strength Formulation 2 and the Phase 3 comparator (Formulation 1) at pH 1.2 (FIG. 14A), 2.0 (QC) (FIG. 14B), 4.5 (FIG. 14C), and 6.8 (FIG. 14D).

FIG. 15 shows an elacestrant dihydrochloride tablet removed from the dissolution medium and cut in half after 9 min.

FIG. 16 is a plot of tablet disintegration times versus tablet hardness for 100 mg and 400 mg elacestrant dihydrochloride tablets.

FIG. 17 is a plot of percent dissolved at 5 minutes versus inverse of tablet hardness (corrected by a factor of 1.68 for 400 mg) for 100 mg and 400 mg elacestrant dihydrochloride tablets in the QC medium.

FIG. 18 is a plot of percent dissolved at 15 minutes versus inverse of tablet hardness (corrected by a factor of 1.68 for 400 mg) for 100 mg and 400 mg elacestrant dihydrochloride tablets in the QC medium.

FIGS. 19A-D show dissolution profiles of elacestrant dihydrochloride tablets (100 mg), Formulation 1, hardness 9.4 kP at multiple pHs (FIG. 19A: pH 1.2, FIG. 19B: pH 2.0 (QC), FIG. 14C: pH 4.5, and FIG. 14D: pH 6.8), as measured and as predicted by a P-PSD HD model. The measured dissolution percentage is represented by the square plots, and the calculated dissolution is represented by the line.

FIGS. 20A-D show dissolution profiles of elacestrant dihydrochloride tablets (400 mg), Formulation 2, hardness 16 kP at multiple pH pHs (FIG. 20A: pH 1.2, FIG. 20B: pH 2.0 (QC), FIG. 20C: pH 4.5, and FIG. 20D: pH 6.8), as measured and as predicted by a P-PSD HD model. The measured dissolution percentage is represented by the square plots, and the calculated dissolution is represented by the line.

FIG. 21 shows dissolution profiles of 100 mg elacestrant dihydrochloride tablets, including VBA and VBB, compared to clinical and technical tablet batches.

FIG. 22 shows dissolution profiles of 400 mg elacestrant dihydrochloride tablets, including VBA and VBB, compared to clinical and technical tablet batches.

FIG. 23 is the predicted average PK profile following OD repeat dosing of 2×100 mg batch VBB in the fed state (low-fat, low-calorie meal).

FIG. 24 is predicted average PK profile following OD repeat dosing of 2×100 mg batch Formulation 1 (Phase 3 comparator) in the fed state (low-fat, low-calorie meal).

FIG. 25 is the predicted average PK profile following OD repeat dosing of 400 mg batch VBB in the fed state (low-fat, low-calorie meal).

FIG. 26 is predicted average PK profile following OD repeat dosing of 400 mg batch Formulation 1 (Phase 3 comparator) in the fed state (low-fat, low-calorie meal).

FIGS. 27A-C show predicted bioavailability (FIG. 27A), gut extraction (FIG. 27B), and liver extraction (FIG. 27C) over 31 clinical scenarios.

FIGS. 28A-C show the effect of food on in vivo dissolution, absorption, and systemic availability for 400 mg tablets of Formulation 1 (Phase 3 comparator) in the fasted state (FIG. 28A), and in the fed state after a low-fat, low-calorie meal (LL) (FIG. 28B) or after a high-fat, high-calorie meal (HH) (FIG. 28C).

FIGS. 29A-B show the effect of food on in vivo dissolution, absorption, and systemic availability for 400 mg tablets of Formulation 2 (16 kP) in the fasted state (FIG. 29A), and in the fed state after a low-fat, low-calorie meal (LL) (FIG. 29B).

FIGS. 30A-B show the effect of food on in vivo dissolution, absorption, and systemic availability for 400 mg tablets of Formulation 2 (23 kP) in the fasted state (FIG. 30A), and in the fed state after a low-fat, low-calorie meal (LL) (FIG. 30B).

FIGS. 31A-B show the effect of food on in vivo dissolution, absorption, and systemic availability for 400 mg tablets of Formulation 2 (16 kP) in the fasted state (FIG. 31A), and in the fed state after a high-fat, high-calorie meal (HH) (FIG. 31B).

FIG. 32 shows predicted bioavailability in the fasted state versus the product of dose and fraction dissolved at 15 min across all formulations.

FIG. 33 shows predicted bioavailability in the fasted state versus the product of dose and fraction dissolved at 15 min across tablets only.

FIG. 34 shows safe space determination for 100 mg elacestrant dihydrochloride tablets.

FIG. 35 shows safe space determination for 400 mg elacestrant dihydrochloride tablets.

### DETAILED DESCRIPTION

In the following description, numerous specific details are set forth in order to provide a thorough understanding of the present technology. Particular exemplary embodiments of the present technology may be implemented without some or all of these specific details. In other instances, well known process operations have not been described in detail in order not to unnecessarily obscure the present technologies.

The present disclosure relates to solid pharmaceutical compositions comprising elacestrant dihydrochloride in tablet form, having a high hardness value and a favorable dissolution profile. The present disclosure also relates to methods of making the solid pharmaceutical compositions and methods of treating breast cancer using the solid pharmaceutical compositions.

The solid pharmaceutical compositions may comprise a core, which is a compressed mixture of the API (elacestrant dihydrochloride) and one or more pharmaceutically acceptable excipients. In some embodiments, the solid pharmaceutical composition comprises a core and a film coating the core. In some embodiments, the core comprises elacestrant dihydrochloride and one or more pharmaceutically acceptable excipients. In some embodiments, all or a portion of the elacestrant dihydrochloride is present in granules (i.e., is intragranular). In some embodiments, the granules further comprise one or more pharmaceutically acceptable excipients. In some embodiments, the core further comprises one or more pharmaceutically acceptable excipients that is not present in the granules (i.e., is extragranular). In some embodiments, the solid pharmaceutical composition does not comprise a film coating the core. In some embodiments, the film is an immediate release (IR) coating film.

### Elacestrant Dihydrochloride (API)

Elacestrant dihydrochloride is an estrogen receptor antagonist, and has the chemical name: (6R)-6-(2-(N-(4-(2-(ethylamino)ethyl)benzyl)-N-ethylamino)-4-methoxyphenyl)-5,6,7,8-tetrahydronaphthalen-2-ol dihydrochloride. The elacestrant dihydrochloride molecular formula is C₃₀H₄₀C₁₂N₂O₂, with a molecular mass of 531.56 g/mol. Elacestrant dihydrochloride is a white to off-white to grey solid and is feely soluble in 0.01 N HCl. The chemical structure of elacestrant dihydrochloride is shown below:

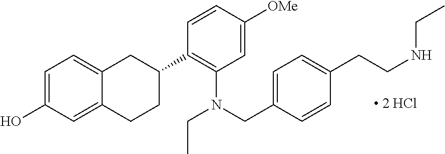 [SMILES: CCNCCc1ccc(CN(CC)c2cc(OC)ccc2[C@@H]2CCc3cc(O)ccc3C2)cc1]

### Structure of Elacestrant Dihydrochloride

Elacestrant (free base) (MW 458.64 g/mol) is an estrogen receptor antagonist that binds to estrogen receptor-alpha (ERα). In ER-positive (ER+) HER2-negative (HER2−) breast cancer cells, elacestrant inhibited 17β-estradiol mediated cell proliferation at concentrations inducing degradation of ERα protein mediated through proteasomal pathway. Elacestrant demonstrated in vitro and in vivo antitumor activity including in ER+HER2-breast cancer models resistant to fulvestrant and cyclin-dependent kinase 4/6 inhibitors and those harboring estrogen receptor 1 gene (ESR1) mutations.

Pharmaceutical compositions according to the present disclosure comprise elacestrant dihydrochloride at a concentration effective to achieve a therapeutic benefit in a human subject. In some embodiments, the pharmaceutical composition comprises elacestrant dihydrochloride at a concentration of greater than or equal to about 30 wt. %, greater than or equal to about 35 wt. %, greater than or equal to about 40 wt. %, greater than or equal to about 41 wt. %, greater than or equal to about 42 wt. %, greater than or equal to about 43 wt. %, greater than or equal to about 44 wt. %, greater than or equal to about 45 wt. %, greater than or equal to about 46 wt. %, greater than or equal to about 47 wt. %, greater than or equal to about 48 wt. %, greater than or equal to about 49 wt. %, greater than or equal to about 50 wt. %, greater than or equal to about 55 wt. %, greater than or equal to about 60 wt. %, or any range or value therein between.

In some embodiments, the pharmaceutical composition comprises elacestrant dihydrochloride at a concentration of less than or equal to about 60 wt. %, less than or equal to about 55 wt. %, less than or equal to about 50 wt. %, less than or equal to about 49 wt. %, less than or equal to about 48 wt. %, less than or equal to about 47 wt. %, less than or equal to about 46 wt. %, less than or equal to about 45 wt. %, less than or equal to about 44 wt. %, less than or equal to about 43 wt. %, less than or equal to about 42 wt. %, less than or equal to about 41 wt. %, less than or equal to about 40 wt. %, less than or equal to about 35 wt. %, less than or equal to about 30 wt. %, or any range or value therein between.

In some embodiments, the pharmaceutical composition comprises elacestrant dihydrochloride at a concentration of about 30 wt. %, about 30.5 wt. %, about 31 wt. %, about 31.5 wt. %, about 32 wt. %, about 32.5 wt. %, about 33 wt. %, about 33.5 wt. %, about 34 wt. %, about 34.5 wt. %, about 35 wt. %, about 35.5 wt. %, about 36 wt. %, about 36.5 wt. %, about 37 wt. %, about 37.5 wt. %, about 38 wt. %, about 38.5 wt. %, about 39 wt. %, about 39.5 wt. %, about 40 wt. %, about 40.5 wt. %, about 41 wt. %, about 41.5 wt. %, about 42 wt. %, about 42.5 wt. %, about 43 wt. %, about 43.5 wt. %, about 44 wt. %, about 44.5 wt. %, about 45 wt. %, about 45.1 wt. %, about 45.2 wt. %, about 45.3 wt. %, about 45.4 wt. %, about 45.5 wt. %, about 45.6 wt. %, about 45.7 wt. %, about 45.8 wt. %, about 45.9 wt. %, about 46 wt. %, about 46.1 wt. %, about 46.2 wt. %, about 46.3 wt. %, about 46.4 wt. %, about 46.5 wt. %, about 46.6 wt. %, about 46.7 wt. %, about 46.8 wt. %, about 46.9 wt. %, about 47 wt. %, about 47.5 wt. %, about 48 wt. %, about 48.5 wt. %, about 49 wt. %, about 49.5 wt. %, about 50 wt. %, about 50.5 wt. %, about 51 wt. %, about 51.5 wt. %, about 52 wt. %, about 52.5 wt. %, about 53 wt. %, about 53.5 wt. %, about 54 wt. %, about 54.5 wt. %, about 55 wt. %, about 55.5 wt. %, about 56 wt. %, about 56.5 wt. %, about 57 wt. %, about 57.5 wt. %, about 58 wt. %, about 58.5 wt. %, about 59 wt. %, about 59.5 wt. %, about 60 wt. %, or any range or value therein between.

In some embodiments, the pharmaceutical composition comprises elacestrant dihydrochloride at a concentration of about 30 wt. % to about 60 wt. %, about 35 wt. % to about 55 wt. %, about 40 wt. % to about 50 wt. %, about 41 wt. % to about 49 wt. %, about 42 wt. % to about 48 wt. %, about 43 wt. % to about 47 wt. %, about 44 wt. % to about 46 wt. %, about 44 wt. % to about 47 wt. %, about 44 wt. % to about 48 wt. %, about 45 wt. % to about 46 wt. %, or any range or value therein.

The elacestrant dihydrochloride may be present at any dose effective to achieve a therapeutic benefit in a human subject. In some embodiments, the elacestrant dihydrochloride is present in an amount of greater than or equal to about 50 mg, greater than or equal to about 55 mg, greater than or equal to about 60 mg, greater than or equal to about 65 mg, greater than or equal to about 70 mg, greater than or equal to about 75 mg, greater than or equal to about 80 mg, greater than or equal to about 85 mg, greater than or equal to about 90 mg, greater than or equal to about 95 mg, greater than or equal to about 100 mg, greater than or equal to about 110 mg, greater than or equal to about 120 mg, greater than or equal to about 130 mg, greater than or equal to about 140 mg, greater than or equal to about 150 mg, greater than or equal to about 160 mg, greater than or equal to about 170 mg, greater than or equal to about 180 mg, greater than or equal to about 190 mg, greater than or equal to about 200 mg, greater than or equal to about 210 mg, greater than or equal to about 220 mg, greater than or equal to about 230 mg, greater than or equal to about 240 mg, greater than or equal to about 250 mg, greater than or equal to about 260 mg, greater than or equal to about 270 mg, greater than or equal to about 280 mg, greater than or equal to about 290 mg, greater than or equal to about 300 mg, greater than or equal to about 310 mg, greater than or equal to about 320 mg, greater than or equal to about 330 mg, greater than or equal to about 340 mg, greater than or equal to about 350 mg, greater than or equal to about 360 mg, greater than or equal to about 370 mg, greater than or equal to about 380 mg, greater than or equal to about 390 mg, greater than or equal to about 400 mg, greater than or equal to about 410 mg, greater than or equal to about 420 mg, greater than or equal to about 430 mg, greater than or equal to about 440 mg, greater than or equal to about 450 mg, greater than or equal to about 460 mg, greater than or equal to about 470 mg, greater than or equal to about 480 mg, greater than or equal to about 490 mg, greater than or equal to about 500 mg, greater than or equal to about 550 mg, greater than or equal to about 600 mg, greater than or equal to about 650 mg, greater than or equal to about 700 mg, greater than or equal to about 750 mg, greater than or equal to about 800 mg, or any range or value therein between.

In some embodiments, the elacestrant dihydrochloride is present in an amount of less than or equal to about 800 mg, less than or equal to about 750 mg, less than or equal to about 700 mg, less than or equal to about 650 mg, less than or equal to about 600 mg, less than or equal to about 550 mg, less than or equal to about 500 mg, less than or equal to about 490 mg, less than or equal to about 480 mg, less than or equal to about 470 mg, less than or equal to about 460 mg, less than or equal to about 450 mg, less than or equal to about 440 mg, less than or equal to about 430 mg, less than or equal to about 420 mg, less than or equal to about 410 mg, less than or equal to about 400 mg, less than or equal to about 390 mg, less than or equal to about 380 mg, less than or equal to about 370 mg, less than or equal to about 360 mg, less than or equal to about 350 mg, less than or equal to about 340 mg, less than or equal to about 330 mg, less than or equal to about 320 mg, less than or equal to about 310 mg, less than or equal to about 300 mg, less than or equal to about 290 mg, less than or equal to about 280 mg, less than or equal to about 270 mg, less than or equal to about 260 mg, less than or equal to about 250 mg, less than or equal to about 240 mg, less than or equal to about 230 mg, less than or equal to about 220 mg, less than or equal to about 210 mg, less than or equal to about 200 mg, less than or equal to about 190 mg, less than or equal to about 180 mg, less than or equal to about 170 mg, less than or equal to about 160 mg, less than or equal to about 150 mg, less than or equal to about 140 mg, less than or equal to about 130 mg, less than or equal to about 120 mg, less than or equal to about 110 mg, less than or equal to about 100 mg, less than or equal to about 95 mg, less than or equal to about 90 mg, less than or equal to about 85 mg, less than or equal to about 80 mg, less than or equal to about 75 mg, less than or equal to about 70 mg, less than or equal to about 65 mg, less than or equal to about 60 mg, less than or equal to about 55 mg, less than or equal to about 50 mg, or any range or value therein between.

In some embodiments, the elacestrant dihydrochloride is present in an amount of about 50 mg to about 800 mg, about 50 mg to about 700 mg, about 50 mg to about 600 mg, about 50 mg to about 500 mg, about 50 mg to about 400 mg, about 50 mg to about 300 mg, about 50 mg to about 200 mg, about 50 mg to about 100 mg, about 100 mg to about 500 mg, about 100 mg to about 400 mg, about 100 mg to about 300 mg, bout 100 mg to about 200 mg, about 150 mg to about 500 mg, about 150 mg to about 400 mg, about 150 mg to about 300 mg, about 150 mg to about 200 mg, about 200 mg to about 500 mg, about 200 mg to about 400 mg, about 200 mg to about 300 mg, about 250 mg to about 500 mg, about 250 mg to about 400 mg, about 250 mg to about 300 mg, about 300 mg to about 500 mg, about 300 mg to about 400 mg, about 60 mg to about 140 mg, about 70 mg to about 130 mg, about 80 mg to about 120 mg, about 90 mg to about 110 mg, about 95 mg to about 105 mg, about 150 mg to about 200 mg, about 160 mg to about 190 mg, about 170 mg to about 180 mg, about 210 mg to about 290 mg, about 220 mg to about 280 mg, about 230 mg to about 270 mg, about 240 mg to about 260 mg, about 250 mg to about 260 mg, about 350 mg to about 450 mg, about 360 mg to about 440 mg, about 370 mg to about 430 mg, about 380 mg to about 420 mg, about 390 mg to about 410 mg, about 395 mg to about 405 mg, or any range or value therein.

In some embodiment, the elacestrant dihydrochloride is present in an amount of about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 140 mg, about 150 mg, about 160 mg, about 170 mg, about 172 mg, about 175 mg, about 180 mg, about 190 mg, about 200 mg, about 210 mg, about 220 mg, about 225 mg, about 230 mg, about 240 mg, about 250 mg, about 258 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, or any range or value therein between.

In some embodiments, the solid pharmaceutical composition comprises a core and a film coating the core. In some embodiments, the core comprises elacestrant dihydrochloride and one or more pharmaceutically acceptable excipients. In some embodiments, all or a portion of the elacestrant dihydrochloride is present in granules (i.e., is intragranular). In some embodiments, the granules further comprise one or more pharmaceutically acceptable excipients. In some embodiments, the core further comprises one or more pharmaceutically acceptable excipients that is not present in the granules (i.e., is extragranular). In some embodiments, the solid pharmaceutical composition does not comprise a film coating the core.

### Excipients

Pharmaceutical compositions according to the present disclosure comprise one or more pharmaceutically acceptable excipients. The pharmaceutically acceptable excipients may include, but are not limited to, binders, disintegrants, fillers, lubricants, glidants, lusters, preservatives, antioxidants, surfactants, coloring agents, and sweeteners.

In some embodiments, the binders may include, but are not limited to: natural polymers (e.g., gum arabic, gelatin, sodium alginate, pullulan, starch, pregelatinized starch, gum tragacanth, etc.); semi-synthetic polymers (e.g., carboxymethylcellulose sodium, dextrin, hydroxyethylcellulose, hydroxypropylcellulose, hypromellose, maltodextrin, methylcellulose, etc.); synthetic polymers (e.g., macrogols (polyethylene glycol), polyvinyl alcohols (PVA), povidone (PVP), copovidone (copolymer of 1-vinyl-2-pyrrolidone and vinyl acetate), etc.); microcrystalline cellulose; and any other suitable binder known in the art.

In some embodiments, the fillers may include, but are not limited to, SMCC (silicified microcrystalline cellulose, composed of microcrystalline cellulose and colloidal silica), starch, pre-gelatinized starch, calcium phosphate, calcium carbonate, sucrose, lactose, maltodextrin, mannitol, sorbitol, sodium chloride, microcrystalline cellulose, and any other suitable filler known in the art. In some embodiments, the filler is a blend of two or more pharmaceutically acceptable excipients. In some embodiments, the filler is a blend comprising microcrystalline cellulose and colloidal silicon dioxide. In some embodiments the filler is a blend consisting essentially of microcrystalline cellulose and colloidal silicon dioxide. In some embodiments the filler is a blend consisting of microcrystalline cellulose and colloidal silicon dioxide. In some embodiments, the filler is SMCC or a blend comprising microcrystalline cellulose and colloidal silicon dioxide. In some embodiments, the filler is a blend comprising about 98 wt. % microcrystalline cellulose and about 2 wt. % colloidal silicon dioxide. In some embodiments, the blend is a granulated form comprising granules that comprise the microcrystalline cellulose and the colloidal silicon dioxide. In some embodiments, the granules comprise about 98 wt. % microcrystalline cellulose and about 2 wt. % colloidal silicon dioxide.

In some embodiments, the filler is SMCC. In some embodiments, the SMCC may be a commercially available SMCC. In some embodiments, the SMCC has an average particle size of about 25 μm to about 500 μm, about 25 μm to about 250 μm, about 25 μm to about 200 μm, about 25 μm to about 150 μm, about 25 μm to about 125 μm, about 25 μm to about 100 μm, about 25 μm to about 65 μm, about 25 μm to about 50 μm, about 50 μm to about 500 μm, about 50 μm to about 250 μm, about 50 μm to about 200 μm, about 50 μm to about 150 μm, about 50 μm to about 125 μm, about 50 μm to about 100 μm, about 50 μm to about 65 μm, about 65 μm to about 500 μm, about 65 μm to about 250 μm, about 65 μm to about 200 μm, about 65 μm to about 150 μm, about 65 μm to about 125 μm, about 65 μm to about 100 μm, about 100 μm to about 500 μm, about 100 μm to about 250 μm, about 100 μm to about 200 μm, about 100 μm to about 150 μm, about 100 μm to about 125 μm, about 125 μm to about 500 μm, about 125 μm to about 250 μm, about 125 μm to about 200 μm, about 125 μm to about 150 μm, about 150 μm to about 500 μm, about 150 μm to about 250 μm, about 150 μm to about 200 μm, about 200 μm to about 500 μm, about 200 μm to about 250 μm, or about 250 μm to about 500 μm. In some embodiments, the SMCC has an average particle size of about 25 μm, about 50 μm, about 100 μm, about 65 μm, about 75 μm about 100 μm, about 125 μm, about 150 μm, about 165 μm, about 200 μm, about 225 μm, about 250 μm, about 300 μm, about 350 μm, about 400 μm, about 450 μm, or about 500 μm.

In some embodiments, the SMCC has a particle size of about 25 μm to about 500 μm, about 25 μm to about 250 μm, about 25 μm to about 200 μm, about 25 μm to about 150 μm, about 25 μm to about 125 μm, about 25 μm to about 100 μm, about 25 μm to about 65 μm, about 25 μm to about 50 μm, about 50 μm to about 500 μm, about 50 μm to about 250 μm, about 50 μm to about 200 μm, about 50 μm to about 150 μm, about 50 μm to about 125 μm, about 50 μm to about 100 μm, about 50 μm to about 65 μm, about 65 μm to about 500 μm, about 65 μm to about 250 μm, about 65 μm to about 200 μm, about 65 μm to about 150 μm, about 65 μm to about 125 μm, about 65 μm to about 100 μm, about 100 μm to about 500 μm, about 100 μm to about 250 μm, about 100 μm to about 200 μm, about 100 μm to about 150 μm, about 100 μm to about 125 μm, about 125 μm to about 500 μm, about 125 μm to about 250 μm, about 125 μm to about 200 μm, about 125 μm to about 150 μm, about 150 μm to about 500 μm, about 150 μm to about 250 μm, about 150 μm to about 200 μm, about 200 μm to about 500 μm, about 200 μm to about 250 μm, or about 250 μm to about 500 μm. In some embodiments, the SMCC has a particle size of about 25 μm, about 50 μm, about 100 μm, about 65 μm, about 75 μm, about 100 μm, about 125 μm, about 150 μm, about 165 μm, about 200 μm, about 225 μm, about 250 μm, about 300 μm, about 350 μm, about 400 μm, about 450 μm, or about 500 μm.

In some embodiments, the SMCC has a bulk density of about 0.05 g/mL to about 1.5 g/mL, about 0.05 g/mL to about 1 g/mL, about 0.05 g/mL to about 0.75 g/mL, about 0.05 g/mL to about 0.6 g/mL, about 0.05 g/mL to about 0.55 g/mL, about 0.05 g/mL to about 0.5 g/mL, about 0.05 g/mL to about 0.39 g/mL, about 0.05 g/mL to about 0.38 g/mL, about 0.05 g/mL to about 0.37 g/mL, about 0.05 g/mL to about 0.27 g/mL, about 0.05 g/mL to about 0.25 g/mL, about 0.05 g/mL to about 0.2 g/mL, about 0.05 g/mL to about 0.15 g/mL, about 0.05 g/mL to about 0.1 g/mL, about 0.1 g/mL to about 1.5 g/mL, about 0.1 g/mL to about 1 g/mL, about 0.1 g/mL to about 0.75 g/mL, about 0.1 g/mL to about 0.6 g/mL, about 0.1 g/mL to about 0.55 g/mL, about 0.1 g/mL to about 0.5 g/mL, about 0.1 g/mL to about 0.39 g/mL, about 0.1 g/mL to about 0.38 g/mL, about 0.1 g/mL to about 0.37 g/mL, about 0.1 g/mL to about 0.27 g/mL, about 0.1 g/mL to about 0.25 g/mL, about 0.1 g/mL to about 0.2 g/mL, about 0.1 g/mL to about 0.15 g/mL, about 0.15 g/mL to about 1.5 g/mL, about 0.15 g/mL to about 1 g/mL, about 0.15 g/mL to about 0.75 g/mL, about 0.15 g/mL to about 0.6 g/mL, about 0.15 g/mL to about 0.55 g/mL, about 0.15 g/mL to about 0.5 g/mL, about 0.15 g/mL to about 0.39 g/mL, 0.15 g/mL to about 0.38 g/mL, about 0.15 g/mL to about 0.37 g/mL, about 0.15 g/mL to about 0.27 g/mL, about 0.15 g/mL to about 0.25 g/mL, about 0.15 g/mL to about 0.2 g/mL, about 0.2 g/mL to about 1.5 g/mL, about 0.2 g/mL to about 1 g/mL, about 0.2 g/mL to about 0.75 g/mL, about 0.2 g/mL to about 0.6 g/mL, about 0.2 g/mL to about 0.55 g/mL, about 0.2 g/mL to about 0.5 g/mL, 0.2 g/mL to about 0.39 g/mL, about 0.2 g/mL to about 0.38 g/mL, about 0.2 g/mL to about 0.37 g/mL, about 0.2 g/mL to about 0.27 g/mL, about 0.2 g/mL to about 0.25 g/mL, about 0.25 g/mL to about 1.5 g/mL, about 0.25 g/mL to about 1 g/mL, about 0.25 g/mL to about 0.75 g/mL, about 0.25 g/mL to about 0.6 g/mL, about 0.25 g/mL to about 0.55 g/mL, about 0.25 g/mL to about 0.5 g/mL, 0.25 g/mL to about 0.39 g/mL, about 0.25 g/mL to about 0.38 g/mL, about 0.25 g/mL to about 0.37 g/mL, about 0.25 g/mL to about 0.27 g/mL, about 0.27 g/mL to about 1.5 g/mL, about 0.27 g/mL to about 1 g/mL, about 0.27 g/mL to about 0.75 g/mL, about 0.27 g/mL to about 0.6 g/mL, about 0.27 g/mL to about 0.55 g/mL, about 0.27 g/mL to about 0.5 g/mL, 0.27 g/mL to about 0.39 g/mL, about 0.27 g/mL to about 0.38 g/mL, about 0.27 g/mL to about 0.37 g/mL, about 0.37 g/mL to about 1.5 g/mL, about 0.37 g/mL to about 1 g/mL, about 0.37 g/mL to about 0.75 g/mL, about 0.37 g/mL to about 0.6 g/mL, about 0.37 g/mL to about 0.55 g/mL, about 0.37 g/mL to about 0.5 g/mL, 0.37 g/mL to about 0.39 g/mL, 0.37 g/mL to about 0.38 g/mL, about 0.38 g/mL to about 1.5 g/mL, about 0.38 g/mL to about 1 g/mL, about 0.38 g/mL to about 0.75 g/mL, about 0.38 g/mL to about 0.6 g/mL, about 0.38 g/mL to about 0.55 g/mL, about 0.38 g/mL to about 0.5 g/mL, about 0.39 g/mL to about 1.5 g/mL, about 0.39 g/mL to about 1 g/mL, about 0.39 g/mL to about 0.75 g/mL, about 0.39 g/mL to about 0.6 g/mL, about 0.39 g/mL to about 0.55 g/mL, about 0.39 g/mL to about 0.5 g/mL, 0.38 g/mL to about 0.39 g/mL, about 0.5 g/mL to about 1.5 g/mL, about 0.5 g/mL to about 1 g/mL, about 0.5 g/mL to about 0.75 g/mL, about 0.5 g/mL to about 0.6 g/mL, about 0.5 g/mL to about 0.55 g/mL, about 0.55 g/mL to about 1.5 g/mL, about 0.55 g/mL to about 1 g/mL, about 0.55 g/mL to about 0.75 g/mL, about 0.55 g/mL to about 0.6 g/mL, about 0.6 g/mL to about 1.5 g/mL, about 0.6 g/mL to about 1 g/mL, about 0.6 g/mL to about 0.75 g/mL, about 0.75 g/mL to about 1.5 g/mL, about 0.75 g/mL to about 1 g/mL, or about 1 g/mL to about 1.5 g/mL. In some embodiments, the SMCC has a bulk density of about 0.1 g/mL, about 0.2 g/mL, about 0.25 g/mL, about 0.27 g/mL, about 0.3 g/mL, about 0.35 g/mL, about 0.37 g/mL, about 0.38 g/mL, about 0.4 g/mL, about 0.45 g/mL, about 0.5 g/mL, about 0.55 g/mL, about 0.6 g/mL, about 0.75 g/mL, about 0.8 g/mL, about 0.85 g/mL, about 0.9 g/mL, about 0.95 g/mL, about 1 g/mL, or about 1.5 g/mL.

In some embodiments, the disintegrants may include, but are not limited to, starch, microcrystalline cellulose, sodium alginate, croscarmellose sodium, crospovidone (crosslinked povidone), sodium starch glycolate, partially pregelatinized starch, and any other suitable disintegrant known in the art.

In some embodiments, the lubricants may include, but are not limited to, magnesium stearate, stearic acid, vegetable stearin, sodium stearyl fumarate, glyceryl di-behenate, talc, silica, polyethylene glycol (e.g., PEG-4000 or PEG-6000), sodium lauryl sulfate (SLS), and any other suitable lubricant known in the art.

In some embodiments, the glidants may include, but are not limited to, colloidal silicon dioxide, talc, starch, ascorbyl palmitate, calcium palmitate, magnesium stearate, and any other suitable glidant known in the art.

In some embodiments, the pharmaceutical composition comprises a film coating the core. In some embodiments, the film is an immediate release (IR) film. The film may comprise any suitable material known in the art, including but not limited to: cellulosic polymers (e.g., hydroxypropyl methyl cellulose (HPMC), hydroxypropyl cellulose (HPC), hydroxyethyl cellulose (HEC), methyl cellulose (MC), sodium carboxymethyl cellulose (NaCMC), etc.); vinyl derivatives (e.g., polyvinyl pyrrolidone (PVP), polyvinylpyrrolidone-polyvinyl acetate copolymers, polyvinyl alcohol (PVA), polyvinyl alcohol-polyethylene glycol copolymers, etc.); acrylic polymers and copolymers (e.g., EUDRAGIT® E, etc.); and glycols (e.g., polyethylene glycols). In some embodiments, the film comprises a commercially-available mixture of polyethylene glycol PVA, and inorganic substances (e.g., OPADRY® II (which includes talc, polyethylene glycol, polyvinyl alcohol, and titanium dioxide)). In some embodiments, the film may be present at a concentration of about 1 wt. % to about 5 wt. % (e.g., about 1 wt. %, about 1.5 wt. %, about 2 wt. %, about 2.5 wt. %, about 3 wt. %, about 3.5 wt. %, about 4 wt. %, about 4 wt. %, or about 5 wt. %), relative to the total weight of the composition. In some embodiments, the pharmaceutical composition does not comprise a film coating the core.

The excipients may be intragranular (e.g., present in granules along with elacestrant dihydrochloride and one or more pharmaceutically acceptable excipients), extragranular (e.g., present external to granules comprising elacestrant dihydrochloride and one or more pharmaceutically acceptable excipients), or a combination of intragranular and extragranular.

Any of the pharmaceutically acceptable excipients may be present in the solid pharmaceutical composition at any suitable concentration for achieving advantageous physical properties (e.g., tablet hardness), manufacturability, or storage stability. In some embodiments, any of the pharmaceutically acceptable excipients (e.g., fillers, binders, disintegrants, glidants, lubricants, etc.) may be present at a concentration of greater than or equal to about 0.01 wt. %, greater than or equal to about 0.05 wt. %, greater than or equal to about 0.1 wt. %, greater than or equal to about 0.2 wt. %, greater than or equal to about 0.3 wt. %, greater than or equal to about 0.4 wt. %, greater than or equal to about 0.5 wt. %, greater than or equal to about 0.6 wt. %, greater than or equal to about 0.7 wt. %, greater than or equal to about 0.8 wt. %, greater than or equal to about 0.9 wt. %, greater than or equal to about 1.0 wt. %, greater than or equal to about 1.2 wt. %, greater than or equal to about 1.4 wt. %, greater than or equal to about 1.5 wt. %, greater than or equal to about 1.6 wt. %, greater than or equal to about 1.8 wt. %, greater than or equal to about 2.0 wt. %, greater than or equal to about 2.2 wt. %, greater than or equal to about 2.4 wt. %, greater than or equal to about 2.5 wt. %, greater than or equal to about 2.6 wt. %, greater than or equal to about 2.8 wt. %, greater than or equal to about 3.0 wt. %, greater than or equal to about 3.2 wt. %, greater than or equal to about 3.4 wt. %, greater than or equal to about 3.5 wt. %, greater than or equal to about 3.6 wt. %, greater than or equal to about 3.8 wt. %, greater than or equal to about 4.0 wt. %, greater than or equal to about 4.2 wt. %, greater than or equal to about 4.4 wt. %, greater than or equal to about 4.5 wt. %, greater than or equal to about 4.6 wt. %, greater than or equal to about 4.8 wt. %, greater than or equal to about 5.0 wt. %, greater than or equal to about 5.5 wt. %, greater than or equal to about 6.0 wt. %, greater than or equal to about 6.5 wt. %, greater than or equal to about 7.0 wt. %, greater than or equal to about 7.5 wt. %, greater than or equal to about 8.0 wt. %, greater than or equal to about 8.5 wt. %, greater than or equal to about 9.0 wt. %, greater than or equal to about 9.5 wt. %, greater than or equal to about 10.0 wt. %, greater than or equal to about 10.5 wt. %, greater than or equal to about 11.0 wt. %, greater than or equal to about 11.5 wt. %, greater than or equal to about 12.0 wt. %, greater than or equal to about 12.5 wt. %, greater than or equal to about 13.0 wt. %, greater than or equal to about 13.5 wt. %, greater than or equal to about 14.0 wt. %, greater than or equal to about 14.5 wt. %, greater than or equal to about 15.0 wt. %, greater than or equal to about 15.5 wt. %, greater than or equal to about 16.0 wt. %, greater than or equal to about 16.5 wt. %, greater than or equal to about 17.0 wt. %, greater than or equal to about 17.5 wt. %, greater than or equal to about 18.0 wt. %, greater than or equal to about 18.5 wt. %, greater than or equal to about 19.0 wt. %, greater than or equal to about 19.5 wt. %, greater than or equal to about 20.0 wt. %, greater than or equal to about 25 wt. %, greater than or equal to about 30 wt. %, greater than or equal to about 35 wt. %, greater than or equal to about 40 wt. %, greater than or equal to about 45 wt. %, greater than or equal to about 50 wt. %, greater than or equal to about 55 wt. %, greater than or equal to about 60 wt. %, greater than or equal to about 65 wt. %, greater than or equal to about 70 wt. %, or any range or value therein between.

In some embodiments, any of the pharmaceutically acceptable excipients may be present at a concentration of less than or equal to about 70 wt. %, less than or equal to about 65 wt. %, less than or equal to about 60 wt. %, less than or equal to about 55 wt. %, less than or equal to about 50 wt. %, less than or equal to about 45 wt. %, less than or equal to about 40 wt. %, less than or equal to about 35 wt. %, less than or equal to about 30 wt. %, less than or equal to about 25 wt. %, less than or equal to about 20 wt. %, less than or equal to about 19.5 wt. %, less than or equal to about 19.0 wt. %, less than or equal to about 18.5 wt. %, less than or equal to about 18.0 wt. %, less than or equal to about 17.5 wt. %, less than or equal to about 17.0 wt. %, less than or equal to about 16.5 wt. %, less than or equal to about 16.0 wt. %, less than or equal to about 15.5 wt. %, less than or equal to about 15.0 wt. %, less than or equal to about 14.5 wt. %, less than or equal to about 14.0 wt. %, less than or equal to about 13.5 wt. %, less than or equal to about 13.0 wt. %, less than or equal to about 12.5 wt. %, less than or equal to about 12.0 wt. %, less than or equal to about 11.5 wt. %, less than or equal to about 11.0 wt. %, less than or equal to about 10.5 wt. %, less than or equal to about 10.0 wt. %, less than or equal to about 9.5 wt. %, less than or equal to about 9.0 wt. %, less than or equal to about 8.5 wt. %, less than or equal to about 8.0 wt. %, less than or equal to about 7.5 wt. %, less than or equal to about 7.0 wt. %, less than or equal to about 6.5 wt. %, less than or equal to about 6.0 wt. %, less than or equal to about 5.5 wt. %, less than or equal to about 5.0 wt. %, less than or equal to about 4.8 wt. %, less than or equal to about 4.6 wt. %, less than or equal to about 4.5 wt. %, less than or equal to about 4.4 wt. %, less than or equal to about 4.2 wt. %, less than or equal to about 4.0 wt. %, less than or equal to about 3.8 wt. %, less than or equal to about 3.6 wt. %, less than or equal to about 3.5 wt. %, less than or equal to about 3.4 wt. %, less than or equal to about 3.2 wt. %, less than or equal to about 3.0 wt. %, less than or equal to about 2.8 wt. %, less than or equal to about 2.6 wt. %, less than or equal to about 2.5 wt. %, less than or equal to about 2.4 wt. %, less than or equal to about 2.2 wt. %, less than or equal to about 2.0 wt. %, less than or equal to about 1.8 wt. %, less than or equal to about 1.6 wt. %, less than or equal to about 1.5 wt. %, less than or equal to about 1.4 wt. %, less than or equal to about 1.2 wt. %, less than or equal to about 1.0 wt. %, less than or equal to about 0.9 wt. %, less than or equal to about 0.8 wt. %, less than or equal to about 0.7 wt. %, less than or equal to about 0.6 wt. %, less than or equal to about 0.5 wt. %, less than or equal to about 0.4 wt. %, less than or equal to about 0.3 wt. %, less than or equal to about 0.2 wt. %, less than or equal to about 0.1 wt. %, less than or equal to about 0.05 wt. %, less than or equal to about 0.01 wt. %, or any range or value therein between.

In some embodiments, any of the pharmaceutically acceptable excipients may be present at a concentration of about 0.01 wt. %, about 0.05 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.2 wt. %, about 1.4 wt. %, about 1.5 wt. %, about 1.6 wt. %, about 1.8 wt. %, about 2.0 wt. %, about 2.2 wt. %, about 2.4 wt. %, about 2.5 wt. %, about 2.6 wt. %, about 2.8 wt. %, about 3.0 wt. %, about 3.2 wt. %, about 3.4 wt. %, about 3.5 wt. %, about 3.6 wt. %, about 3.8 wt. %, about 4.0 wt. %, about 4.2 wt. %, about 4.4 wt. %, about 4.5 wt. %, about 4.6 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, or any range or value therein between.

In some embodiments, any of the pharmaceutically acceptable excipients may be present at a concentration of about 0.01 wt. % to about 70.0 wt. %, about 0.01 wt. % to about 60.0 wt. %, about 0.01 wt. % to about 50.0 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 5 wt. %, about 0.01 wt. % to about 1 wt. %, about 0.1 wt. % to about 50.0 wt. %, about 0.1 wt. % to about 40 wt. %, about 0.1 wt. % to about 30 wt. %, about 0.1 wt. % to about 20 wt. %, about 0.1 wt. % to about 15 wt. %, about 0.1 wt. % to about 10 wt. %, about 0.1 wt. % to about 5 wt. %, about 0.1 wt. % to about 1 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 15 wt. %, about 1 wt. % to about 10 wt. %, about 1 wt. % to about 5 wt. %, about 1 wt. % to about 2 wt. %, about 5 wt. % to about 70 wt. %, about 5 wt. % to about 60 wt. %, about 5 wt. % to about 50 wt. %, about 5 wt. % to about 40 wt. %, about 5 wt. % to about 30 wt. %, about 5 wt. % to about 20 wt. %, about 5 wt. % to about 15 wt. %, about 5 wt. % to about 10 wt. %, about 10 wt. % to about 70 wt. %, about 10 wt. % to about 60 wt. %, about 10 wt. % to about 50 wt. %, about 10 wt. % to about 40 wt. %, about 10 wt. % to about 30 wt. %, about 10 wt. % to about 20 wt. %, about 10 wt. % to about 15 wt. %, or any range or value therein between.

In some embodiments, the composition comprises one or more of a filler, a binder, a disintegrant, a glidant, or a lubricant. In some embodiments, the composition comprises two or more of a filler, a binder, a disintegrant, a glidant, or a lubricant. In some embodiments, the composition comprises three or more of a filler, a binder, a disintegrant, a glidant, or a lubricant. In some embodiments, the composition comprises four or more of a filler, a binder, a disintegrant, a glidant, or a lubricant. In some embodiments, the composition comprises a filler, a binder, a disintegrant, a glidant, and a lubricant.

In some embodiments, the composition comprises SMCC and one or more pharmaceutically acceptable excipients. In some embodiments, the composition comprises SMCC and one or more pharmaceutically acceptable excipients selected from microcrystalline cellulose, crospovidone, colloidal silicon dioxide, or magnesium stearate. In some embodiments, the composition comprises SMCC and two or more pharmaceutically acceptable excipients selected from microcrystalline cellulose, crospovidone, colloidal silicon dioxide, or magnesium stearate. In some embodiments, the composition comprises SMCC and three or more pharmaceutically acceptable excipients selected from microcrystalline cellulose, crospovidone, colloidal silicon dioxide, or magnesium stearate. In some embodiments, the composition comprises SMCC, microcrystalline cellulose, crospovidone, colloidal silicon dioxide, and magnesium stearate.

In some embodiments, the composition comprises a blend comprising microcrystalline cellulose and colloidal silicon dioxide and one or more pharmaceutically acceptable excipients. In some embodiments, the composition comprises a blend comprising microcrystalline cellulose and colloidal silicon dioxide and one or more pharmaceutically acceptable excipients selected from microcrystalline cellulose, crospovidone, colloidal silicon dioxide, or magnesium stearate. In some embodiments, the composition comprises a blend comprising microcrystalline cellulose and colloidal silicon dioxide and two or more pharmaceutically acceptable excipients selected from microcrystalline cellulose, crospovidone, colloidal silicon dioxide, or magnesium stearate. In some embodiments, the composition comprises a blend comprising microcrystalline cellulose and colloidal silicon dioxide and three or more pharmaceutically acceptable excipients selected from microcrystalline cellulose, crospovidone, colloidal silicon dioxide, or magnesium stearate. In some embodiments, the composition comprises a blend comprising microcrystalline cellulose and colloidal silicon dioxide, microcrystalline cellulose, crospovidone, colloidal silicon dioxide, and magnesium stearate. In some embodiments, the blend is a granulated form comprising granules that comprise microcrystalline cellulose and colloidal silicon dioxide. In some embodiments, the blend is a granulated form comprising granules that comprise microcrystalline cellulose and colloidal silicon dioxide and one or more pharmaceutically acceptable excipients selected from crospovidone, colloidal silicon dioxide, or magnesium stearate.

In some embodiments, all or a portion of any pharmaceutically acceptable excipient may be present in granules (i.e., is intragranular). In some embodiments, any of the one or more pharmaceutically acceptable excipients may be present in the core but not in granules (i.e., is extragranular). In some embodiments, any of the one or more pharmaceutically acceptable excipients may be intragranular or extragranular.

In some embodiments, an intragranular portion of any pharmaceutically acceptable excipient may be present at a concentration of about 0.01 wt. % to about 70.0 wt. %, about 0.01 wt. % to about 60.0 wt. %, about 0.01 wt. % to about 50.0 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 5 wt. %, about 0.01 wt. % to about 1 wt. %, about 0.1 wt. % to about 50.0 wt. %, about 0.1 wt. % to about 40 wt. %, about 0.1 wt. % to about 30 wt. %, about 0.1 wt. % to about 20 wt. %, about 0.1 wt. % to about 15 wt. %, about 0.1 wt. % to about 10 wt. %, about 0.1 wt. % to about 5 wt. %, about 0.1 wt. % to about 1 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 15 wt. %, about 1 wt. % to about 10 wt. %, about 1 wt. % to about 5 wt. %, about 1 wt. % to about 2 wt. %, about 5 wt. % to about 70 wt. %, about 5 wt. % to about 60 wt. %, about 5 wt. % to about 50 wt. %, about 5 wt. % to about 40 wt. %, about 5 wt. % to about 30 wt. %, about 5 wt. % to about 20 wt. %, about 5 wt. % to about 15 wt. %, about 5 wt. % to about 10 wt. %, about 10 wt. % to about 70 wt. %, about 10 wt. % to about 60 wt. %, about 10 wt. % to about 50 wt. %, about 10 wt. % to about 40 wt. %, about 10 wt. % to about 30 wt. %, about 10 wt. % to about 20 wt. %, about 10 wt. % to about 15 wt. %, or any range or value therein between. In some embodiments, the intragranular portion of any pharmaceutically acceptable excipient may be present at a concentration of about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, about 20 wt. %, about 25 wt. %, or about 30 wt. %.

In some embodiments, an extragranular portion of any pharmaceutically acceptable excipient may be present at a concentration of about 0.01 wt. % to about 70.0 wt. %, about 0.01 wt. % to about 60.0 wt. %, about 0.01 wt. % to about 50.0 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 5 wt. %, about 0.01 wt. % to about 1 wt. %, about 0.1 wt. % to about 50.0 wt. %, about 0.1 wt. % to about 40 wt. %, about 0.1 wt. % to about 30 wt. %, about 0.1 wt. % to about 20 wt. %, about 0.1 wt. % to about 15 wt. %, about 0.1 wt. % to about 10 wt. %, about 0.1 wt. % to about 5 wt. %, about 0.1 wt. % to about 1 wt. %, about 1 wt. % to about 70 wt. %, about 1 wt. % to about 60 wt. %, about 1 wt. % to about 50 wt. %, about 1 wt. % to about 40 wt. %, about 1 wt. % to about 30 wt. %, about 1 wt. % to about 20 wt. %, about 1 wt. % to about 15 wt. %, about 1 wt. % to about 10 wt. %, about 1 wt. % to about 5 wt. %, about 1 wt. % to about 2 wt. %, about 5 wt. % to about 70 wt. %, about 5 wt. % to about 60 wt. %, about 5 wt. % to about 50 wt. %, about 5 wt. % to about 40 wt. %, about 5 wt. % to about 30 wt. %, about 5 wt. % to about 20 wt. %, about 5 wt. % to about 15 wt. %, about 5 wt. % to about 10 wt. %, about 10 wt. % to about 70 wt. %, about 10 wt. % to about 60 wt. %, about 10 wt. % to about 50 wt. %, about 10 wt. % to about 40 wt. %, about 10 wt. % to about 30 wt. %, about 10 wt. % to about 20 wt. %, about 10 wt. % to about 15 wt. %, or any range or value therein between. In some embodiments, the extragranular portion of any pharmaceutically acceptable excipient may be present at a concentration of about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, about 20 wt. %, about 25 wt. %, or about 30 wt. %.

In some embodiments, the composition comprises SMCC and one or more additional pharmaceutically acceptable excipients. In some embodiments, the composition comprises SMCC and one or more pharmaceutically acceptable excipients selected from microcrystalline cellulose, crospovidone, colloidal silicon dioxide, or magnesium stearate. In some embodiments, the composition comprises SMCC, microcrystalline cellulose, crospovidone, colloidal silicon dioxide, and magnesium stearate.

In some embodiments, the composition comprises about 30 wt. % to about 60 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises about 40 wt. % to about 50 wt. % elacestrant dihydrochloride; about 23 wt. % to about 28 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises about 40 wt. % to about 50 wt. % elacestrant dihydrochloride; about 20 wt. % to about 30 wt. % microcrystalline cellulose; about 5 wt. % to about 8 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises about 40 wt. % to about 50 wt. % elacestrant dihydrochloride; about 23 wt. % to about 28 wt. % microcrystalline cellulose; about 5 wt. % to about 10 wt. % crospovidone; about 1 wt. % to about 2 wt. % magnesium stearate; about 15 wt. % to about 25 wt. % SMCC; and about 0.1 wt. % to about 0.3 wt. % colloidal silicon dioxide.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 16 wt. % to about 22 wt. % SMCC; and about 0.1 wt. % to about 0.3 wt. % colloidal silicon dioxide.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises about 42 wt. % to about 47 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises about 42 wt. % to about 47 wt. % elacestrant dihydrochloride; about 20 wt. % to about 30 wt. % microcrystalline cellulose; about 5 wt. % to about 9 wt. % crospovidone; about 0.5 wt. % to about 2.5 wt. % magnesium stearate; about 15 wt. % to about 22 wt. % SMCC; and about 0.05 wt. % to about 0.5 wt. % colloidal silicon dioxide.

In some embodiments, the composition comprises about 42 wt. % to about 47 wt. % elacestrant dihydrochloride; about 23 wt. % to about 28 wt. % microcrystalline cellulose; about 5 wt. % to about 8 wt. % crospovidone; about 1 wt. % to about 2 wt. % magnesium stearate; about 16 wt. % to about 22 wt. % SMCC; and about 0.1 wt. % to about 0.3 wt. % colloidal silicon dioxide.

In some embodiments, the composition comprises about 45.9 wt. % elacestrant dihydrochloride, about 26.4 wt. % microcrystalline cellulose, about 6.4 wt. % crospovidone, about 1.6 wt. % magnesium stearate, about 19.5 wt. % SMCC, and about 0.2 wt. % colloidal silicon dioxide.

In some embodiments, the composition comprises about 30 wt. % to about 60 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; about 0.01 wt. % to about 1 wt. % colloidal silicon dioxide, and about 1 wt. % to about 5 wt. % of a film.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; about 0.01 wt. % to about 1 wt. % colloidal silicon dioxide, and about 1 wt. % to about 5 wt. % of a film.

In some embodiments, the composition comprises about 40 wt. % to about 50 wt. % elacestrant dihydrochloride; about 23 wt. % to about 28 wt. % microcrystalline cellulose; about 5 wt. % to about 10 wt. % crospovidone; about 0.5 wt. % to about 3 wt. % magnesium stearate; about 15 wt. % to about 25 wt. % SMCC; about 0.1 wt. % to about 0.3 wt. % colloidal silicon dioxide, and about 1 wt. % to about 5 wt. % of a film.

In some embodiments, the composition comprises about 40 wt. % to about 50 wt. % elacestrant dihydrochloride; about 20 wt. % to about 30 wt. % microcrystalline cellulose; about 5 wt. % to about 8 wt. % crospovidone; about 0.5 wt. % to about 3 wt. % magnesium stearate; about 15 wt. % to about 25 wt. % SMCC; about 0.1 wt. % to about 0.3 wt. % colloidal silicon dioxide, and about 1 wt. % to about 5 wt. % of a film.

In some embodiments, the composition comprises about 40 wt. % to about 50 wt. % elacestrant dihydrochloride; about 23 wt. % to about 28 wt. % microcrystalline cellulose; about 5 wt. % to about 10 wt. % crospovidone; about 1 wt. % to about 2 wt. % magnesium stearate; about 15 wt. % to about 25 wt. % SMCC; about 0.1 wt. % to about 0.3 wt. % colloidal silicon dioxide, and about 1 wt. % to about 5 wt. % of a film.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 16 wt. % to about 22 wt. % SMCC; about 0.1 wt. % to about 0.3 wt. % colloidal silicon dioxide, and about 1 wt. % to about 5 wt. % of a film.

In some embodiments, the composition comprises about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; about 0.1 wt. % to about 0.3 wt. % colloidal silicon dioxide, and about 1 wt. % to about 5 wt. % of a film.

In some embodiments, the composition comprises about 42 wt. % to about 47 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; about 0.01 wt. % to about 1 wt. % colloidal silicon dioxide, and about 1 wt. % to about 5 wt. % of a film.

In some embodiments, the composition comprises about 42 wt. % to about 47 wt. % elacestrant dihydrochloride; about 20 wt. % to about 30 wt. % microcrystalline cellulose; about 5 wt. % to about 9 wt. % crospovidone; about 0.5 wt. % to about 2.5 wt. % magnesium stearate; about 15 wt. % to about 22 wt. % SMCC; about 0.05 wt. % to about 0.5 wt. % colloidal silicon dioxide, and about 1 wt. % to about 5 wt. % of a film.

In some embodiments, the composition comprises about 42 wt. % to about 47 wt. % elacestrant dihydrochloride; about 23 wt. % to about 28 wt. % microcrystalline cellulose; about 5 wt. % to about 8 wt. % crospovidone; about 1 wt. % to about 2 wt. % magnesium stearate; about 16 wt. % to about 22 wt. % SMCC; about 0.1 wt. % to about 0.3 wt. % colloidal silicon dioxide, and about 1 wt. % to about 5 wt. % of a film.

In some embodiments, the composition comprises about 45.9 wt. % elacestrant dihydrochloride, about 26.4 wt. % microcrystalline cellulose, about 6.4 wt. % crospovidone, about 1.6 wt. % magnesium stearate, about 19.5 wt. % SMCC, about 0.2 wt. % colloidal silicon dioxide and about 3 wt. % of a film.

In some embodiments, the composition comprises a core comprising about 30 wt. % to about 60 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises a core comprising about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises a core comprising about 40 wt. % to about 50 wt. % elacestrant dihydrochloride; about 23 wt. % to about 28 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises a core comprising about 40 wt. % to about 50 wt. % elacestrant dihydrochloride; about 20 wt. % to about 30 wt. % microcrystalline cellulose; about 5 wt. % to about 8 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises a core comprising about 40 wt. % to about 50 wt. % elacestrant dihydrochloride; about 23 wt. % to about 28 wt. % microcrystalline cellulose; about 5 wt. % to about 10 wt. % crospovidone; about 1 wt. % to about 2 wt. % magnesium stearate; about 15 wt. % to about 25 wt. % SMCC; and about 0.1 wt. % to about 0.3 wt. % colloidal silicon dioxide.

In some embodiments, the composition comprises a core comprising about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 16 wt. % to about 22 wt. % SMCC; and about 0.1 wt. % to about 0.3 wt. % colloidal silicon dioxide.

In some embodiments, the composition comprises a core comprising about 40 wt. % to about 50 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.1 wt. % to about 0.3 wt. % colloidal silicon dioxide.

In some embodiments, the composition comprises a core comprising about 42 wt. % to about 47 wt. % elacestrant dihydrochloride; about 20 wt. % to about 30 wt. % microcrystalline cellulose; about 5 wt. % to about 10 wt. % crospovidone; about 0.5 wt. % to about 3 wt. % magnesium stearate; about 15 wt. % to about 25 wt. % SMCC; and about 0.01 wt. % to about 1 wt. % colloidal silicon dioxide.

In some embodiments, the composition comprises a core comprising about 42 wt. % to about 47 wt. % elacestrant dihydrochloride; about 20 wt. % to about 30 wt. % microcrystalline cellulose; about 5 wt. % to about 9 wt. % crospovidone; about 0.5 wt. % to about 2.5 wt. % magnesium stearate; about 15 wt. % to about 22 wt. % SMCC; and about 0.05 wt. % to about 0.5 wt. % colloidal silicon dioxide.

In some embodiments, the composition comprises a core comprising about 42 wt. % to about 47 wt. % elacestrant dihydrochloride; about 23 wt. % to about 28 wt. % microcrystalline cellulose; about 5 wt. % to about 8 wt. % crospovidone; about 1 wt. % to about 2 wt. % magnesium stearate; about 16 wt. % to about 22 wt. % SMCC; and about 0.1 wt. % to about 0.3 wt. % colloidal silicon dioxide.

In some embodiments, the composition comprises a core comprising about 45.9 wt. % elacestrant dihydrochloride, about 26.4 wt. % microcrystalline cellulose, about 6.4 wt. % crospovidone, about 1.6 wt. % magnesium stearate, about 19.5 wt. % SMCC, and about 0.2 wt. % colloidal silicon dioxide.

In some embodiments, the composition comprises: a core comprising about 30 wt. % to about 60 wt. % elacestrant dihydrochloride, about 20 wt. % to about 30 wt. % microcrystalline cellulose, about 5 wt. % to about 10 wt. % crospovidone, about 0.5 wt. % to about 3 wt. % magnesium stearate, about 15 wt. % to about 25 wt. % SMCC, and about 0.01 wt. % to about 1 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 20 wt. % to about 30 wt. % microcrystalline cellulose, about 5 wt. % to about 10 wt. % crospovidone, about 0.5 wt. % to about 3 wt. % magnesium stearate, about 15 wt. % to about 25 wt. % SMCC, and about 0.01 wt. % to about 1 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 23 wt. % to about 28 wt. % microcrystalline cellulose, about 5 wt. % to about 10 wt. % crospovidone, about 0.5 wt. % to about 3 wt. % magnesium stearate, about 15 wt. % to about 25 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 20 wt. % to about 30 wt. % microcrystalline cellulose, about 5 wt. % to about 8 wt. % crospovidone, about 0.5 wt. % to about 3 wt. % magnesium stearate, about 15 wt. % to about 25 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 23 wt. % to about 28 wt. % microcrystalline cellulose, about 5 wt. % to about 10 wt. % crospovidone, about 1 wt. % to about 2 wt. % magnesium stearate, about 15 wt. % to about 25 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 20 wt. % to about 30 wt. % microcrystalline cellulose, about 5 wt. % to about 10 wt. % crospovidone, about 0.5 wt. % to about 3 wt. % magnesium stearate, about 16 wt. % to about 22 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 40 wt. % to about 50 wt. % elacestrant dihydrochloride, about 20 wt. % to about 30 wt. % microcrystalline cellulose, about 5 wt. % to about 10 wt. % crospovidone, about 0.5 wt. % to about 3 wt. % magnesium stearate, about 15 wt. % to about 25 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 42 wt. % to about 47 wt. % elacestrant dihydrochloride, about 20 wt. % to about 30 wt. % microcrystalline cellulose, about 5 wt. % to about 10 wt. % crospovidone, about 0.5 wt. % to about 3 wt. % magnesium stearate, about 15 wt. % to about 25 wt. % SMCC, and about 0.01 wt. % to about 1 wt. % colloidal silicon dioxide; and a film in an amount of about 1 wt. % to about 5 wt. % that coats the core.

In some embodiments, the composition comprises: a core comprising about 42 wt. % to about 47 wt. % elacestrant dihydrochloride, about 20 wt. % to about 30 wt. % microcrystalline cellulose, about 5 wt. % to about 9 wt. % crospovidone, about 0.5 wt. % to about 2.5 wt. % magnesium stearate, about 15 wt. % to about 22 wt. % SMCC, and about 0.05 wt. % to about 0.5 wt. % colloidal silicon dioxide; and a film in an amount of about 1 wt. % to about 5 wt. % that coats the core.

In some embodiments, the composition comprises: a core comprising about 42 wt. % to about 47 wt. % elacestrant dihydrochloride, about 23 wt. % to about 28 wt. % microcrystalline cellulose, about 5 wt. % to about 8 wt. % crospovidone, about 1 wt. % to about 2 wt. % magnesium stearate, about 16 wt. % to about 22 wt. % SMCC, and about 0.1 wt. % to about 0.3 wt. % colloidal silicon dioxide; and a film in an amount of about 1 wt. % to about 5 wt. % that coats the core.

In some embodiments, the composition comprises: a core comprising about 45.9 wt. % elacestrant dihydrochloride, about 26.4 wt. % microcrystalline cellulose, about 6.4 wt. % crospovidone, about 1.6 wt. % magnesium stearate, about 19.5 wt. % SMCC, and about 0.2 wt. % colloidal silicon dioxide; and a film in an amount of about 3 wt. % that coats the core.

In some embodiments, the composition comprises one or more of intragranular SMCC, intragranular microcrystalline cellulose, intragranular crospovidone, and intragranular magnesium stearate. In some embodiments, the composition comprises one or more of extragranular SMCC, extragranular crospovidone, extragranular colloidal silicon dioxide, and extragranular magnesium stearate. In some embodiments, the composition comprises both intragranular SMCC and extragranular SMCC. In some embodiments, the composition comprises both intragranular crospovidone and extragranular crospovidone. In some embodiments, the composition comprises both intragranular magnesium stearate and extragranular magnesium stearate. In some embodiments, the composition comprises intragranular SMCC, intragranular microcrystalline cellulose, intragranular crospovidone, intragranular magnesium stearate, extragranular SMCC, extragranular crospovidone, extragranular colloidal silicon dioxide, and extragranular magnesium stearate.

In some embodiments, the composition comprises about 30 wt. % to about 60 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate.

In some embodiments, the composition comprises about 40 wt. % to about 50 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate.

In some embodiments, the composition comprises about 40 wt. % to about 50 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate.

In some embodiments, the composition comprises about 42 wt. % to about 47 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate.

In some embodiments, the composition comprises about 45.9 wt. % intragranular elacestrant dihydrochloride, about 26.4 wt. % intragranular microcrystalline cellulose, about 4.1 wt. % intragranular crospovidone, about 8 wt. % intragranular SMCC, about 0.5 wt. % intragranular magnesium stearate, about 2.3 wt. % extragranular crospovidone, about 11.5 wt. % extragranular SMCC, about 0.2 wt. % extragranular colloidal silicon dioxide, and about 1.1 wt. % extragranular magnesium stearate.

In some embodiments, the composition comprises about 30 wt. % to about 60 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, about 0.1 wt. % to about 2 wt. % extragranular magnesium stearate, and about 1 wt. % to about 5 wt. % of a film.

In some embodiments, the composition comprises about 40 wt. % to about 50 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, about 0.1 wt. % to about 2 wt. % extragranular magnesium stearate, and about 1 wt. % to about 5 wt. % of a film.

In some embodiments, the composition comprises about 40 wt. % to about 50 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, about 0.5 wt. % to about 2 wt. % extragranular magnesium stearate, and about 1 wt. % to about 5 wt. % of a film.

In some embodiments, the composition comprises about 42 wt. % to about 47 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, about 0.5 wt. % to about 2 wt. % extragranular magnesium stearate, and about 1 wt. % to about 5 wt. % of a film.

In some embodiments, the composition comprises about 45.9 wt. % intragranular elacestrant dihydrochloride, about 26.4 wt. % intragranular microcrystalline cellulose, about 4.1 wt. % intragranular crospovidone, about 8 wt. % intragranular SMCC, about 0.5 wt. % intragranular magnesium stearate, about 2.3 wt. % extragranular crospovidone, about 11.5 wt. % extragranular SMCC, about 0.2 wt. % extragranular colloidal silicon dioxide, about 1.1 wt. % extragranular magnesium stearate, and about 3 wt. % of a film.

In some embodiments, the composition comprises: a core comprising about 30 wt. % to about 60 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate.

In some embodiments, the composition comprises: a core comprising about 40 wt. % to about 50 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate.

In some embodiments, the composition comprises: a core comprising about 40 wt. % to about 50 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate.

In some embodiments, the composition comprises: a core comprising about 42 wt. % to about 47 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate.

In some embodiments, the composition comprises: a core comprising about 45.9 wt. % intragranular elacestrant dihydrochloride, about 26.4 wt. % intragranular microcrystalline cellulose, about 4.1 wt. % intragranular crospovidone, about 8 wt. % intragranular SMCC, about 0.5 wt. % intragranular magnesium stearate, about 2.3 wt. % extragranular crospovidone, about 11.5 wt. % extragranular SMCC, about 0.2 wt. % extragranular colloidal silicon dioxide, and about 1.1 wt. % extragranular magnesium stearate.

In some embodiments, the composition comprises: a core comprising about 30 wt. % to about 60 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate; and a film in an amount of about 1 wt. % to about 5 wt. % that coats the core.

In some embodiments, the composition comprises: a core comprising about 40 wt. % to about 50 wt. % intragranular elacestrant dihydrochloride, about 20 wt. % to about 35 wt. % intragranular microcrystalline cellulose, about 1 wt. % to about 8 wt. % intragranular crospovidone, about 2 wt. % to about 12 wt. % intragranular SMCC, about 0.1 wt. % to about 1.5 wt. % intragranular magnesium stearate, about 1 wt. % to about 10 wt. % extragranular crospovidone, about 5 wt. % to about 20 wt. % extragranular SMCC, about 0.05 wt. % to about 1 wt. % extragranular colloidal silicon dioxide, and about 0.1 wt. % to about 2 wt. % extragranular magnesium stearate; and a film in an amount of about 1 wt. % to about 5 wt. % that coats the core.

In some embodiments, the composition comprises: a core comprising about 40 wt. % to about 50 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate; and a film in an amount of about 1 wt. % to about 5 wt. % that coats the core.

In some embodiments, the composition comprises: a core comprising about 42 wt. % to about 47 wt. % intragranular elacestrant dihydrochloride, about 20 wt. % to about 30 wt. % intragranular microcrystalline cellulose, about 2 wt. % to about 6 wt. % intragranular crospovidone, about 5 wt. % to about 10 wt. % intragranular SMCC, about 0.1 wt. % to about 1 wt. % intragranular magnesium stearate, about 1 wt. % to about 5 wt. % extragranular crospovidone, about 5 wt. % to about 15 wt. % extragranular SMCC, about 0.1 wt. % to about 1 wt. % extragranular colloidal silicon dioxide, and about 0.5 wt. % to about 2 wt. % extragranular magnesium stearate; and a film in an amount of about 1 wt. % to about 5 wt. % that coats the core.

In some embodiments, the composition comprises: a core comprising about 45.9 wt. % intragranular elacestrant dihydrochloride, about 26.4 wt. % intragranular microcrystalline cellulose, about 4.1 wt. % intragranular crospovidone, about 8 wt. % intragranular SMCC, about 0.5 wt. % intragranular magnesium stearate, about 2.3 wt. % extragranular crospovidone, about 11.5 wt. % extragranular SMCC, about 0.2 wt. % extragranular colloidal silicon dioxide, and about 1.1 wt. % extragranular magnesium stearate; and a film in an amount of about 3 wt. % that coats the core.

In some embodiments, the composition may be a composition prepared by any of the methods of manufacturing solid pharmaceutical compositions disclosed herein.

### Tablet Hardness

Pharmaceutical compositions according to the present disclosure may have any suitable hardness for achieving a favorable dissolution profile and target bioavailability. Methods of measuring tablet hardness will be known to those of ordinary skill in the art.

In some embodiments, the solid pharmaceutical composition is a tablet that is compressed to a hardness of greater than or equal to about 5.0 kP, greater than or equal to about 5.1 kP, greater than or equal to about 5.2 kP, greater than or equal to about 5.3 kP, greater than or equal to about 5.4 kP, greater than or equal to about 5.5 kP, greater than or equal to about 5.6 kP, greater than or equal to about 5.7 kP, greater than or equal to about 5.8 kP, greater than or equal to about 5.9 kP, greater than or equal to about 6.0 kP, greater than or equal to about 6.1 kP, greater than or equal to about 6.2 kP, greater than or equal to about 6.3 kP, greater than or equal to about 6.4 kP, greater than or equal to about 6.5 kP, greater than or equal to about 6.6 kP, greater than or equal to about 6.7 kP, greater than or equal to about 6.8 kP, greater than or equal to about 6.9 kP, greater than or equal to about 7.0 kP, greater than or equal to about 7.1 kP, greater than or equal to about 7.2 kP, greater than or equal to about 7.3 kP, greater than or equal to about 7.4 kP, greater than or equal to about 7.5 kP, greater than or equal to about 7.6 kP, greater than or equal to about 7.7 kP, greater than or equal to about 7.8 kP, greater than or equal to about 7.9 kP, greater than or equal to about 8.0 kP, greater than or equal to about 8.1 kP, greater than or equal to about 8.2 kP, greater than or equal to about 8.3 kP, greater than or equal to about 8.4 kP, greater than or equal to about 8.5 kP, greater than or equal to about 8.6 kP, greater than or equal to about 8.7 kP, greater than or equal to about 8.8 kP, greater than or equal to about 8.9 kP, greater than or equal to about 9.0 kP, greater than or equal to about 9.1 kP, greater than or equal to about 9.2 kP, greater than or equal to about 9.3 kP, greater than or equal to about 9.4 kP, greater than or equal to about 9.5 kP, greater than or equal to about 9.6 kP, greater than or equal to about 9.7 kP, greater than or equal to about 9.8 kP, greater than or equal to about 9.9 kP, greater than or equal to about 10.0 kP, greater than or equal to about 10.5 kP, greater than or equal to about 11.0 kP, greater than or equal to about 11.5 kP, greater than or equal to about 12.0 kP, greater than or equal to about 12.5 kP, greater than or equal to about 13.0 kP, greater than or equal to about 13.5 kP, greater than or equal to about 14.0 kP, greater than or equal to about 14.5 kP, greater than or equal to about 15.0 kP, greater than or equal to about 15.5 kP, greater than or equal to about 16.0 kP, greater than or equal to about 16.5 kP, greater than or equal to about 17.0 kP, greater than or equal to about 17.5 kP, greater than or equal to about 18.0 kP, greater than or equal to about 18.5 kP, greater than or equal to about 19.0 kP, greater than or equal to about 19.5 kP, greater than or equal to about 20.0 kP, greater than or equal to about 20.5 kP, greater than or equal to about 21.0 kP, greater than or equal to about 21.5 kP, greater than or equal to about 22.0 kP, greater than or equal to about 22.5 kP, greater than or equal to about 23.0 kP, greater than or equal to about 23.5 kP, greater than or equal to about 24.0 kP, greater than or equal to about 24.5 kP, greater than or equal to about 25.0 kP, greater than or equal to about 25.5 kP, greater than or equal to about 26.0 kP, greater than or equal to about 26.5 kP, greater than or equal to about 27.0 kP, greater than or equal to about 27.5 kP, greater than or equal to about 28.0 kP, greater than or equal to about 28.5 kP, greater than or equal to about 29.0 kP, greater than or equal to about 29.5 kP, greater than or equal to about 30.0 kP, greater than or equal to about 30.5 kP, greater than or equal to about 31.0 kP, greater than or equal to about 31.5 kP, greater than or equal to about 32.0 kP, greater than or equal to about 32.5 kP, greater than or equal to about 33.0 kP, greater than or equal to about 33.5 kP, greater than or equal to about 34.0 kP, greater than or equal to about 34.5 kP, greater than or equal to about 35.0 kP, greater than or equal to about 36.0 kP, greater than or equal to about 37.0 kP, greater than or equal to about 38.0 kP, greater than or equal to about 39.0 kP, greater than or equal to about 40.0 kP, or any range or value therein between.

In some embodiments, the solid pharmaceutical composition is a tablet that is compressed to a hardness of less than or equal to about 5.5 kP, less than or equal to about 5.6 kP, less than or equal to about 5.7 kP, less than or equal to about 5.8 kP, less than or equal to about 5.9 kP, less than or equal to about 6.0 kP, less than or equal to about 6.1 kP, less than or equal to about 6.2 kP, less than or equal to about 6.3 kP, less than or equal to about 6.4 kP, less than or equal to about 6.5 kP, less than or equal to about 6.6 kP, less than or equal to about 6.7 kP, less than or equal to about 6.8 kP, less than or equal to about 6.9 kP, less than or equal to about 7.0 kP, less than or equal to about 7.1 kP, less than or equal to about 7.2 kP, less than or equal to about 7.3 kP, less than or equal to about 7.4 kP, less than or equal to about 7.5 kP, less than or equal to about 7.6 kP, less than or equal to about 7.7 kP, less than or equal to about 7.8 kP, less than or equal to about 7.9 kP, less than or equal to about 8.0 kP, less than or equal to about 8.1 kP, less than or equal to about 8.2 kP, less than or equal to about 8.3 kP, less than or equal to about 8.4 kP, less than or equal to about 8.5 kP, less than or equal to about 8.6 kP, less than or equal to about 8.7 kP, less than or equal to about 8.8 kP, less than or equal to about 8.9 kP, less than or equal to about 9.0 kP, less than or equal to about 9.1 kP, less than or equal to about 9.2 kP, less than or equal to about 9.3 kP, less than or equal to about 9.4 kP, less than or equal to about 9.5 kP, less than or equal to about 9.6 kP, less than or equal to about 9.7 kP, less than or equal to about 9.8 kP, less than or equal to about 9.9 kP, less than or equal to about 10.0 kP, less than or equal to about 10.5 kP, less than or equal to about 11.0 kP, less than or equal to about 11.5 kP, less than or equal to about 12.0 kP, less than or equal to about 12.5 kP, less than or equal to about 13.0 kP, less than or equal to about 13.5 kP, less than or equal to about 14.0 kP, less than or equal to about 14.5 kP, less than or equal to about 15.0 kP, less than or equal to about 15.5 kP, less than or equal to about 16.0 kP, less than or equal to about 16.5 kP, less than or equal to about 17.0 kP, less than or equal to about 17.5 kP, less than or equal to about 18.0 kP, less than or equal to about 18.5 kP, less than or equal to about 19.0 kP, less than or equal to about 19.5 kP, less than or equal to about 20.0 kP, less than or equal to about 20.5 kP, less than or equal to about 21.0 kP, less than or equal to about 21.5 kP, less than or equal to about 22.0 kP, less than or equal to about 22.5 kP, less than or equal to about 23.0 kP, less than or equal to about 23.5 kP, less than or equal to about 24.0 kP, less than or equal to about 24.5 kP, less than or equal to about 25.0 kP, less than or equal to about 25.5 kP, less than or equal to about 26.0 kP, less than or equal to about 26.5 kP, less than or equal to about 27.0 kP, less than or equal to about 27.5 kP, less than or equal to about 28.0 kP, less than or equal to about 28.5 kP, less than or equal to about 29.0 kP, less than or equal to about 29.5 kP, less than or equal to about 30.0 kP, less than or equal to about 30.5 kP, less than or equal to about 31.0 kP, less than or equal to about 31.5 kP, less than or equal to about 32.0 kP, less than or equal to about 32.5 kP, less than or equal to about 33.0 kP, less than or equal to about 33.5 kP, less than or equal to about 34.0 kP, less than or equal to about 34.5 kP, less than or equal to about 35.0 kP, less than or equal to about 36.0 kP, less than or equal to about 37.0 kP, less than or equal to about 38.0 kP, less than or equal to about 39.0 kP, less than or equal to about 40.0 kP, or any range or value therein between.

Tablet hardness may be determined using any suitable method known to those in the art. For instance, in some embodiments, tablet hardness may be determined using the methods described in Resistance to Crushing of Tablets, EUROPEAN PHARMACOPOEIA 2.9.8 or Tablet Breaking Force, USP <1217>.

In some embodiments, the tablet hardness may be different for different compositions. For instance, in some embodiments, tablets comprising 100 mg of elacestrant dihydrochloride may have a hardness of about 5.4 to about 16 kP, or of about 9 kP, as measured in accordance with USP <1217>. In some embodiments, tablets comprising 400 mg of elacestrant dihydrochloride may have a hardness of about 12 to about 23 kP, or of about 17 kP, as measured in accordance with USP <1217>.

### Dissolution Profile

Some solid elacestrant dihydrochloride compositions have been observed as forming a gelling layer on their surface upon contact with an aqueous medium. Without being bound by any theory, this phenomenon may slow the permeation of water into the solid composition-slowing the dissolution of the composition and altering drug release. Solid pharmaceutical compositions according to the present disclosure have a suitable dissolution profile to ensure bioavailability of elacestrant, to achieve a desired therapeutic effect. The release of elacestrant dihydrochloride from the solid pharmaceutical compositions may be measured according to any suitable method known in the art. In some embodiments, a suitable dissolution test apparatus and methods is described in current editions of USP <711> and USP <1092>, using apparatus 2 (paddles) and the HPLC procedure described therein. In some embodiments, the dissolution profile of the composition is measured with the USP2 apparatus (paddles), at a stirring speed of 75 rpm, a temperature of 37° C., in 500 mL (for 100 mg tablets) or 1000 mL (for 400 mg tablets) of a medium of 0.01N HCl, with 5 mL samples (no medium replacement) taken through a 10-micron porous filter and characterized by HPLC. In some embodiments, the dissolution profile of the composition is measured for six single tablets with USP2 apparatus, at a stirring speed of 75 rpm from 0 to 45 minutes and a stirring speed of 250 rpm at 45 minutes onwards, a temperature of 37° C., in 500 mL (for 100 mg tablets) or 1000 mL (for 400 mg tablets) of a medium of 0.01N HCl, with 5 mL samples (no medium replacement) taken through a 10-micron porous filter and characterized by HPLC.

In some embodiments, the solid pharmaceutical composition exhibits an elacestrant dihydrochloride dissolution profile in which at least about 70%, at least about 75%, at least about 80%, at least about 85%, or at least about 90% of the elacestrant dihydrochloride is released after 45 min. in a dissolution medium having a pH of 6.8 or less (such as about 6.5 or less, about 6.0 or less, about 5.5 or less, about 5.0 or less, about 4.5 or less, about 4.0 or less, about 3.5 or less, about 3.0 or less, about 2.5 or less, about 2.0 or less, about 1.5 or less, or about 1.2 or less).

In some embodiments, the solid pharmaceutical composition exhibits an elacestrant dihydrochloride dissolution profile in which at least about 25%, at least about 30%, at least about 35%, at least about 40%, at least about 45%, at least about 50%, at least about 55%, at least about 60%, at least about 65%, at least about 70%, at least about 75%, at least about 80%, or at least about 85% of the elacestrant dihydrochloride is released after 15 min. in a dissolution medium having a pH of 6.8 or less (such as about 6.5 or less, about 6.0 or less, about 5.5 or less, about 5.0 or less, about 4.5 or less, about 4.0 or less, about 3.5 or less, about 3.0 or less, about 2.5 or less, about 2.0 or less, about 1.5 or less, or about 1.2 or less).

In some embodiments, the solid pharmaceutical composition exhibits an elacestrant dihydrochloride dissolution profile in which at least about 10%, at least about 11%, at least about 12%, at least about 13%, at least about 14%, at least about 15%, at least about 20%, at least about 25%, at least about 30%, or at least about 35% of the elacestrant dihydrochloride is released after 5 min. in a dissolution medium having a pH of 6.8 or less (such as about 6.5 or less, about 6.0 or less, about 5.5 or less, about 5.0 or less, about 4.5 or less, about 4.0 or less, about 3.5 or less, about 3.0 or less, about 2.5 or less, about 2.0 or less, about 1.5 or less, or about 1.2 or less).

### Methods of Making Solid Pharmaceutical Compositions

Some solid elacestrant dihydrochloride solid compositions have been observed as being difficult to manufacture. For instance, some compositions may have issues at the roller compaction stage, whereby the composition accumulates on the roller, increasing the torque beyond a safe operating limit. Further, some compositions may have issues during tablet compression, whereby material accumulates on the dies and tablet tooling-resulting in increased friction and increasing the compression forces beyond a safe operating limit. The compositions disclosed herein may reduce such manufacturing issues and allow for consistent commercial scale manufacture.

In another aspect, which may be combined with any other aspect or embodiment, the present disclosure relates to methods of making solid pharmaceutical compositions comprising elacestrant dihydrochloride, the methods comprising: compressing a mixture comprising elacestrant dihydrochloride and one or more pharmaceutically acceptable excipients to produce a tablet, wherein the mixture comprises 30 wt. % to 60 wt. % of elacestrant dihydrochloride, relative to the total weight of the mixture; and the compressing is performed at a pressure of at least 1 kN, and the tablet has a hardness of at least 5 kP.

The compressing may be carried out at any suitable pressure to achieve any tablet hardness and/or any dissolution profile discussed above. In some embodiments the compressing is performed at a pressure of greater than or equal to about 1 kN, greater than or equal to about 1.5 kN, greater than or equal to about 2.0 kN, greater than or equal to about 2.5 kN, greater than or equal to about 3.0 kN, greater than or equal to about 3.5 kN, greater than or equal to about 4.0 kN, greater than or equal to about 4.5 kN, greater than or equal to about 5.0 kN, greater than or equal to about 5.5 kN, greater than or equal to about 6.0 kN, greater than or equal to about 6.5 kN, greater than or equal to about 7.0 kN, greater than or equal to about 7.5 kN, greater than or equal to about 8.0 kN, greater than or equal to about 8.5 kN, greater than or equal to about 9.0 kN, greater than or equal to about 9.5 kN, greater than or equal to about 10.0 kN, greater than or equal to about 10.5 kN, greater than or equal to about 11.0 kN, greater than or equal to about 11.5 kN, greater than or equal to about 12.0 kN, greater than or equal to about 12.5 kN, greater than or equal to about 13.0 kN, greater than or equal to about 13.5 kN, greater than or equal to about 14.0 kN, greater than or equal to about 14.5 kN, greater than or equal to about 15.0 kN, greater than or equal to about 15.5 kN, greater than or equal to about 16.0 kN, greater than or equal to about 16.5 kN, greater than or equal to about 17.0 kN, greater than or equal to about 17.5 kN, greater than or equal to about 18.0 kN, greater than or equal to about 18.5 kN, greater than or equal to about 19.0 kN, greater than or equal to about 19.5 kN, greater than or equal to about 20.0 kN, greater than or equal to about 20.5 kN, greater than or equal to about 21.0 kN, greater than or equal to about 21.5 kN, greater than or equal to about 22.0 kN, greater than or equal to about 22.5 kN, greater than or equal to about 23.0 kN, greater than or equal to about 23.5 kN, greater than or equal to about 24.0 kN, greater than or equal to about 24.5 kN, greater than or equal to about 25.0 kN, greater than or equal to about 25.5 kN, greater than or equal to about 26.0 kN, greater than or equal to about 26.5 kN, greater than or equal to about 27.0 kN, greater than or equal to about 27.5 kN, greater than or equal to about 28.0 kN, greater than or equal to about 28.5 kN, greater than or equal to about 29.0 kN, greater than or equal to about 29.5 kN, greater than or equal to about 30.0 kN, greater than or equal to about 30.5 kN, greater than or equal to about 31.0 kN, greater than or equal to about 31.5 kN, greater than or equal to about 32.0 kN, greater than or equal to about 32.5 kN, greater than or equal to about 33.0 kN, greater than or equal to about 33.5 kN, greater than or equal to about 34.0 kN, greater than or equal to about 35.0 kN, greater than or equal to about 36 kN, greater than or equal to about 37 kN, greater than or equal to about 38 kN, greater than or equal to about 39 kN, greater than or equal to about 40 kN, greater than or equal to about 45 kN, or any range or value therein between.

In some embodiments the compressing is performed at a pressure of less than or equal to about 1 kN, less than or equal to about 1.5 kN, less than or equal to about 2.0 kN, less than or equal to about 2.5 kN, less than or equal to about 3.0 kN, less than or equal to about 3.5 kN, less than or equal to about 4.0 kN, less than or equal to about 4.5 kN, less than or equal to about 5.0 kN, less than or equal to about 5.5 kN, less than or equal to about 6.0 kN, less than or equal to about 6.5 kN, less than or equal to about 7.0 kN, less than or equal to about 7.5 kN, less than or equal to about 8.0 kN, less than or equal to about 8.5 kN, less than or equal to about 9.0 kN, less than or equal to about 9.5 kN, less than or equal to about 10.0 kN, less than or equal to about 10.5 kN, less than or equal to about 11.0 kN, less than or equal to about 11.5 kN, less than or equal to about 12.0 kN, less than or equal to about 12.5 kN, less than or equal to about 13.0 kN, less than or equal to about 13.5 kN, less than or equal to about 14.0 kN, less than or equal to about 14.5 kN, less than or equal to about 15.0 kN, less than or equal to about 15.5 kN, less than or equal to about 16.0 kN, less than or equal to about 16.5 kN, less than or equal to about 17.0 kN, less than or equal to about 17.5 kN, less than or equal to about 18.0 kN, less than or equal to about 18.5 kN, less than or equal to about 19.0 kN, less than or equal to about 19.5 kN, less than or equal to about 20.0 kN, less than or equal to about 20.5 kN, less than or equal to about 21.0 kN, less than or equal to about 21.5 kN, less than or equal to about 22.0 kN, less than or equal to about 22.5 kN, less than or equal to about 23.0 kN, less than or equal to about 23.5 kN, less than or equal to about 24.0 kN, less than or equal to about 24.5 kN, less than or equal to about 25.0 kN, less than or equal to about 25.5 kN, less than or equal to about 26.0 kN, less than or equal to about 26.5 kN, less than or equal to about 27.0 kN, less than or equal to about 27.5 kN, less than or equal to about 28.0 kN, less than or equal to about 28.5 kN, less than or equal to about 29.0 kN, less than or equal to about 29.5 kN, less than or equal to about 30.0 kN, less than or equal to about 30.5 kN, less than or equal to about 31.0 kN, less than or equal to about 31.5 kN, less than or equal to about 32.0 kN, less than or equal to about 32.5 kN, less than or equal to about 33.0 kN, less than or equal to about 33.5 kN, less than or equal to about 34.0 kN, less than or equal to about 35.0 kN, less than or equal to about 36 kN, less than or equal to about 37 kN, less than or equal to about 38 kN, less than or equal to about 39 kN, less than or equal to about 40 kN, less than or equal to about 45 kN, or any range or value therein between.

In some embodiments, the compressing may comprise both a pre-compression and a main compression. In some embodiments, pre-compression is performed prior to the main compression. In some embodiments, pre-compression is performed immediately prior to the main compression.

In some embodiments the main compression is performed at a pressure of greater than or equal to about 1 kN, greater than or equal to about 1.5 kN, greater than or equal to about 2.0 kN, greater than or equal to about 2.5 kN, greater than or equal to about 3.0 kN, greater than or equal to about 3.5 kN, greater than or equal to about 4.0 kN, greater than or equal to about 4.5 kN, greater than or equal to about 5.0 kN, greater than or equal to about 5.5 kN, greater than or equal to about 6.0 kN, greater than or equal to about 6.5 kN, greater than or equal to about 7.0 kN, greater than or equal to about 7.5 kN, greater than or equal to about 8.0 kN, greater than or equal to about 8.5 kN, greater than or equal to about 9.0 kN, greater than or equal to about 9.5 kN, greater than or equal to about 10.0 kN, greater than or equal to about 10.5 kN, greater than or equal to about 11.0 kN, greater than or equal to about 11.5 kN, greater than or equal to about 12.0 kN, greater than or equal to about 12.5 kN, greater than or equal to about 13.0 kN, greater than or equal to about 13.5 kN, greater than or equal to about 14.0 kN, greater than or equal to about 14.5 kN, greater than or equal to about 15.0 kN, greater than or equal to about 15.5 kN, greater than or equal to about 16.0 kN, greater than or equal to about 16.5 kN, greater than or equal to about 17.0 kN, greater than or equal to about 17.5 kN, greater than or equal to about 18.0 kN, greater than or equal to about 18.5 kN, greater than or equal to about 19.0 kN, greater than or equal to about 19.5 kN, greater than or equal to about 20.0 kN, greater than or equal to about 20.5 kN, greater than or equal to about 21.0 kN, greater than or equal to about 21.5 kN, greater than or equal to about 22.0 kN, greater than or equal to about 22.5 kN, greater than or equal to about 23.0 kN, greater than or equal to about 23.5 kN, greater than or equal to about 24.0 kN, greater than or equal to about 24.5 kN, greater than or equal to about 25.0 kN, greater than or equal to about 25.5 kN, greater than or equal to about 26.0 kN, greater than or equal to about 26.5 kN, greater than or equal to about 27.0 kN, greater than or equal to about 27.5 kN, greater than or equal to about 28.0 kN, greater than or equal to about 28.5 kN, greater than or equal to about 29.0 kN, greater than or equal to about 29.5 kN, greater than or equal to about 30.0 kN, greater than or equal to about 30.5 kN, greater than or equal to about 31.0 kN, greater than or equal to about 31.5 kN, greater than or equal to about 32.0 kN, greater than or equal to about 32.5 kN, greater than or equal to about 33.0 kN, greater than or equal to about 33.5 kN, greater than or equal to about 34.0 kN, greater than or equal to about 35.0 kN, greater than or equal to about 36 kN, greater than or equal to about 37 kN, greater than or equal to about 38 kN, greater than or equal to about 39 kN, greater than or equal to about 40 kN, greater than or equal to about 45 kN, or any range or value therein between.

In some embodiments the main compression is performed at a pressure of less than or equal to about 1 kN, less than or equal to about 1.5 kN, less than or equal to about 2.0 kN, less than or equal to about 2.5 kN, less than or equal to about 3.0 kN, less than or equal to about 3.5 kN, less than or equal to about 4.0 kN, less than or equal to about 4.5 kN, less than or equal to about 5.0 kN, less than or equal to about 5.5 kN, less than or equal to about 6.0 kN, less than or equal to about 6.5 kN, less than or equal to about 7.0 kN, less than or equal to about 7.5 kN, less than or equal to about 8.0 kN, less than or equal to about 8.5 kN, less than or equal to about 9.0 kN, less than or equal to about 9.5 kN, less than or equal to about 10.0 kN, less than or equal to about 10.5 kN, less than or equal to about 11.0 kN, less than or equal to about 11.5 kN, less than or equal to about 12.0 kN, less than or equal to about 12.5 kN, less than or equal to about 13.0 kN, less than or equal to about 13.5 kN, less than or equal to about 14.0 kN, less than or equal to about 14.5 kN, less than or equal to about 15.0 kN, less than or equal to about 15.5 kN, less than or equal to about 16.0 kN, less than or equal to about 16.5 kN, less than or equal to about 17.0 kN, less than or equal to about 17.5 kN, less than or equal to about 18.0 kN, less than or equal to about 18.5 kN, less than or equal to about 19.0 kN, less than or equal to about 19.5 kN, less than or equal to about 20.0 kN, less than or equal to about 20.5 kN, less than or equal to about 21.0 kN, less than or equal to about 21.5 kN, less than or equal to about 22.0 kN, less than or equal to about 22.5 kN, less than or equal to about 23.0 kN, less than or equal to about 23.5 kN, less than or equal to about 24.0 kN, less than or equal to about 24.5 kN, less than or equal to about 25.0 kN, less than or equal to about 25.5 kN, less than or equal to about 26.0 kN, less than or equal to about 26.5 kN, less than or equal to about 27.0 kN, less than or equal to about 27.5 kN, less than or equal to about 28.0 kN, less than or equal to about 28.5 kN, less than or equal to about 29.0 kN, less than or equal to about 29.5 kN, less than or equal to about 30.0 kN, less than or equal to about 30.5 kN, less than or equal to about 31.0 kN, less than or equal to about 31.5 kN, less than or equal to about 32.0 kN, less than or equal to about 32.5 kN, less than or equal to about 33.0 kN, less than or equal to about 33.5 kN, less than or equal to about 34.0 kN, less than or equal to about 35.0 kN, less than or equal to about 36 kN, less than or equal to about 37 kN, less than or equal to about 38 kN, less than or equal to about 39 kN, less than or equal to about 40 kN, less than or equal to about 45 kN, or any range or value therein between.

In some embodiments the pre-compression is performed at a pressure of about 5% to about 30%, about 5% to about 25%, about 5% to about 20%, about 5% to about 15%, about 5% to about 10%, about 10% to about 30%, about 10% to about 25%, about 10% to about 20%, about 10% to about 15%, about 15% to about 30%, about 15% to about 25%, about 15% to about 20%, about 20% to about 30%, about 20% to about 25%, or about 25% to about 30% of the pressure that the main compression is performed at. In some embodiments the pre-compression is performed at a pressure of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, or about 30% of the pressure that the main compression is performed at.

In some embodiments the pre-compression is performed at a pressure of about 0.1 kN to about 10 kN, about 0.1 kN to about 9 kN, about 0.1 kN to about 8 kN, about 0.1 kN to about 7 kN, about 0.1 kN to about 6 kN, about 0.1 kN to about 5 kN, about 0.1 kN to about 4 kN, about 0.1 kN to about 3 kN, about 0.1 kN to about 2 kN, about 0.1 kN to about 1 kN, 0.1 kN to about 0.5 kN, about 0.5 kN to about 10 kN, about 0.5 kN to about 9 kN, about 0.5 kN to about 8 kN, about 0.5 kN to about 7 kN, about 0.5 kN to about 6 kN, about 0.5 kN to about 5 kN, about 0.5 kN to about 4 kN, about 0.5 kN to about 3 kN, about 0.5 kN to about 2 kN, about 0.5 kN to about 1 kN, about 1 kN to about 10 kN, about 1 kN to about 9 kN, about 1 kN to about 8 kN, about 1 kN to about 7 kN, about 1 kN to about 6 kN, about 1 kN to about 5 kN, about 1 kN to about 4 kN, about 1 kN to about 3 kN, about 1 kN to about 2 kN, 2 kN to about 10 kN, about 2 kN to about 9 kN, about 2 kN to about 8 kN, about 2 kN to about 7 kN, about 2 kN to about 6 kN, about 2 kN to about 5 kN, about 2 kN to about 4 kN, about 2 kN to about 3 kN, 3 kN to about 10 kN, about 3 kN to about 9 kN, about 3 kN to about 8 kN, about 3 kN to about 7 kN, about 3 kN to about 6 kN, about 3 kN to about 5 kN, about 3 kN to about 4 kN, 4 kN to about 10 kN, about 4 kN to about 9 kN, about 4 kN to about 8 kN, about 4 kN to about 7 kN, about 4 kN to about 6 kN, about 4 kN to about 5 kN, 5 kN to about 10 kN, about 5 kN to about 9 kN, about 5 kN to about 8 kN, about 5 kN to about 7 kN, about 5 kN to about 6 kN, 6 kN to about 10 kN, about 6 kN to about 9 kN, about 6 kN to about 8 kN, about 6 kN to about 7 kN, 7 kN to about 10 kN, about 7 kN to about 9 kN, about 7 kN to about 8 kN, about 8 kN to about 10 kN, about 8 kN to about 9 kN, or about 9 kN to about 10 kN. In some embodiments the pre-compression is performed at a pressure of about 0.1 kN, about 0.25 kN, about 0.5 kN, about 0.75 kN, about 1 kN, about 1.5 kN, about 2 kN, about 2.5 kN, about 3 kN, about 3.5 kN, about 4 kN, about 4.5 kN, about 5 kN, about 5.5 kN, about 6 kN, about 6.5 kN, about 7 kN, about 7.5 kN, about 8 kN, about 8.5 kN, about 8.5 kN, about 9 kN, about 9.5 kN, or about 10 kN.

The methods of making solid pharmaceutical compositions according to the present disclosure may include additional steps. For instance, the methods of some embodiments may further comprise granulating an intragranular blend comprising elacestrant dihydrochloride and one or more pharmaceutically acceptable excipients to provide an intragranular phase. The granulation may be performed by any such method known to a person skilled in the art. In some embodiments, the granulation may be a wet granulation process (such as a moisture activated process), a dry granulation process (such as a roller compaction process), or a melt granulation process. In some embodiments, the granulation is a roller compaction process. In some embodiments, the elacestrant dihydrochloride and one or more pharmaceutically acceptable excipients undergo a roller compaction dry granulation process to produce an intragranular phase, after which a compression mixture is formed by adding additional extragranular excipients to the extragranular phase, followed by the compression step and (optionally) coating the resulting tablet with a film (e.g., an immediate release film).

In some embodiments, the method of making a solid pharmaceutical composition comprises mixing elacestrant dihydrochloride and one or more pharmaceutically acceptable excipients to provide an intragranular blend. In some embodiments, the method of making a solid pharmaceutical composition comprises mixing elacestrant dihydrochloride and a filler to provide an intragranular blend. In some embodiments, the method of making a solid pharmaceutical composition comprises mixing an intragranular blend with a lubricant to give a lubricated intragranular blend. In some embodiments, the method of making a solid pharmaceutical composition comprises compacting a lubricated intragranular blend to provide an intragranular phase that comprises granules comprising the elacestrant dihydrochloride, the one or more pharmaceutically acceptable excipients, and the lubricant. In some embodiments, the method of making a solid pharmaceutical composition comprises mixing the intragranular phase with one or more additional pharmaceutically acceptable excipients to provide a compression blend. In some embodiments, the method of making a solid pharmaceutical composition comprises mixing the compression blend with a lubricant to provide a compression mixture. In some embodiments, the method of making a solid pharmaceutical composition comprises compressing a compression mixture to provide a tablet. In some embodiments, the method of making a solid pharmaceutical composition comprises coating a tablet with a film. In some embodiments, the mixture further comprises one or more of a binder, a disintegrant, a glidant, or a lubricant. In some embodiments, the mixture further comprises two or more of a binder, a disintegrant, a glidant, or a lubricant. In some embodiments, the mixture further comprises three or more of a binder, a disintegrant, a glidant, or a lubricant. In some embodiments, the mixture further comprises a binder, a disintegrant, a glidant, and a lubricant. In some embodiments, the method of making a solid pharmaceutical composition comprises: mixing elacestrant dihydrochloride, a first portion of a filler, microcrystalline cellulose, and a first portion of crospovidone to provide an intragranular blend; mixing the intragranular blend with a first portion of magnesium stearate to give a lubricated intragranular blend; compacting the lubricated intragranular blend to provide an intragranular phase comprising granules; mixing the intragranular phase with a second portion of the filler, a second portion of crospovidone, and colloidal silicon dioxide to provide a compression blend; mixing the compression blend with a second portion of magnesium stearate to provide the mixture; compressing the mixture to produce a tablet; and coating the tablet with a film to give the composition. In some embodiments, the granules comprise each of the elacestrant dihydrochloride, the first portion of the filler, the microcrystalline cellulose, the first portion of crospovidone, and the first portion of magnesium stearate as intra granular components. In some embodiments, the second portion of the filler, the second portion of crospovidone, the colloidal silicon dioxide, and the second portion of magnesium stearate are extragranular components. In some embodiments the filler is SMCC or a blend comprising microcrystalline cellulose and colloidal silicon dioxide.

In some embodiments, the method of making a solid pharmaceutical composition comprises combining microcrystalline cellulose and colloidal silicon dioxide to provide the filler prior to the compacting of the intragranular blend. In some embodiments, the combining comprises adding the colloidal silicon dioxide to an aqueous slurry of the microcrystalline cellulose to provide a mixed slurry; and spray drying the mixed slurry to provide the SMCC. In some embodiments, the combining comprises mixing the microcrystalline cellulose and the colloidal silicon dioxide to provide a blend comprising the microcrystalline cellulose and the colloidal silicon dioxide. In some embodiments, the combining comprises granulating the microcrystalline cellulose and the colloidal silicon dioxide to provide a blend comprising granules that comprises the microcrystalline cellulose and the colloidal silicon dioxide. The granulation may be performed by any such method known to a person skilled in the art. In some embodiments, the granulation may be a wet granulation process (such as a moisture activated process), a dry granulation process (such as a roller compaction process), or a melt granulation process. In some embodiments, the granulation is a roller compaction process.

In some embodiments, the method of making a solid pharmaceutical composition comprises: mixing elacestrant dihydrochloride, a first portion of SMCC, microcrystalline cellulose, and a first portion of crospovidone to provide an intragranular blend; mixing the intragranular blend with a first portion of magnesium stearate to give a lubricated intragranular blend; compacting the lubricated intragranular blend to provide an intragranular phase comprising granules; mixing the intragranular phase with a second portion of SMCC, a second portion of crospovidone, and colloidal silicon dioxide to provide a compression blend; mixing the compression blend with a second portion of magnesium stearate to provide the mixture; compressing the mixture to produce a tablet; and coating the tablet with a film to give the composition. In some embodiments, the granules comprise each of the elacestrant dihydrochloride, the first portion of SMCC, the microcrystalline cellulose, the first portion of crospovidone, and the first portion of magnesium stearate as intra granular components. In some embodiments, the second portion of SMCC, the second portion of crospovidone, the colloidal silicon dioxide, and the second portion of magnesium stearate are extragranular components.

In some embodiments, the method of making a solid pharmaceutical composition comprises: mixing elacestrant dihydrochloride, a first portion of a blend comprising microcrystalline cellulose and colloidal silicon dioxide, microcrystalline cellulose, and a first portion of crospovidone to provide an intragranular blend; mixing the intragranular blend with a first portion of magnesium stearate to give a lubricated intragranular blend; compacting the lubricated intragranular blend to provide an intragranular phase comprising granules; mixing the intragranular phase with a second portion of the blend comprising microcrystalline cellulose and colloidal silicon dioxide, a second portion of crospovidone, and colloidal silicon dioxide to provide a compression blend; mixing the compression blend with a second portion of magnesium stearate to provide the mixture; compressing the mixture to produce a tablet; and coating the tablet with a film to give the composition. In some embodiments, the granules comprise each of the elacestrant dihydrochloride, the first portion of the blend comprising microcrystalline cellulose and colloidal silicon dioxide, the microcrystalline cellulose, the first portion of crospovidone, and the first portion of magnesium stearate as intra granular components. In some embodiments, the second portion of the blend comprising microcrystalline cellulose and colloidal silicon dioxide, the second portion of crospovidone, the colloidal silicon dioxide, and the second portion of magnesium stearate are extragranular components.

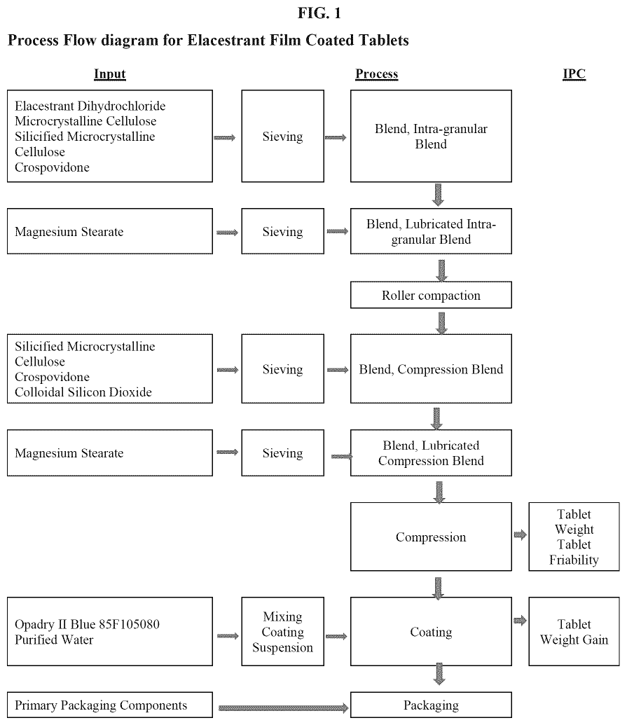

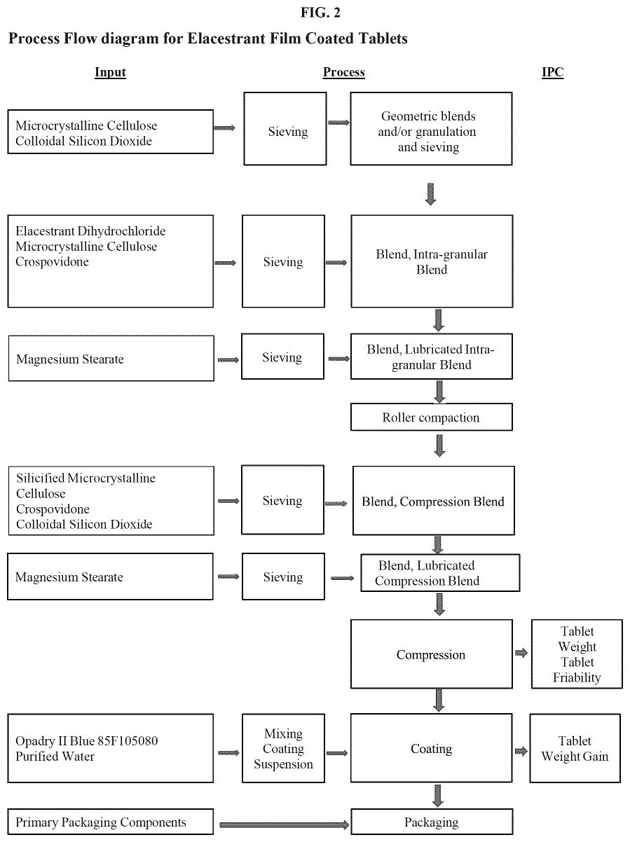

In some embodiments, the method of making a solid pharmaceutical composition comprises the steps as shown in FIG. 1. In some embodiments, the method of making a solid pharmaceutical composition comprises the steps as shown in FIG. 2.

### Methods of Treatment

In another aspect, which may be combined with any other aspect or embodiment, the present disclosure relates to methods of treating breast cancer using elacestrant dihydrochloride solid pharmaceutical compositions. The mechanisms of action for elacestrant dihydrochloride pharmaceutical compositions in treating breast cancer are described in, e.g., U.S. Pat. Nos. 10,745,343; 10,420,734; 10,071,066; and 9,421,264, the entire disclosures of which are hereby incorporated by reference herein. Methods for treating breast cancer using elacestrant dihydrochloride is used interactable, throughout the descriptions and claims, with elacestrant dihydrochloride for use in methods for treating cancer.

As used herein, the term “administration” of an agent to a subject includes any route of introducing or delivering the agent to a subject to perform its intended function. Administration can be carried out by any suitable non-oral route, including, but not limited to, intravenously, intramuscularly, intraperitoneally, subcutaneously, orally, and other suitable routes as described herein. Administration includes self-administration and the administration by another.

As used herein, the term “effective amount” or “therapeutically effective amount” refers to a quantity of an active ingredient or a salt thereof sufficient to achieve a desired effect or a desired therapeutic effect.

In some embodiments, the present disclosure relates to methods for treating breast cancer in a subject, the methods comprising administering to the subject an effective amount of the solid pharmaceutical composition according to any of the embodiments disclosed herein. In some embodiments, the breast cancer is an estrogen receptor positive (ER+) breast cancer and/or a human epidermal growth factor receptor 2 (HER2)-negative (HER2−) breast cancer. In some embodiments, the ER+ breast cancer is an estrogen receptor alpha positive (ERα+) breast cancer. In some embodiments, the breast cancer is an ER+ and estrogen receptor 1 (ESR1)-mutated breast cancer. In some embodiments, the breast cancer is an ERα+ and estrogen receptor 1 (ESR1)-mutated breast cancer. In some embodiments, the breast cancer is ER+/HER2− and estrogen receptor 1 (ESR1)-mutated breast cancer. In some embodiments, the breast cancer is ERα+/HER2− and estrogen receptor 1 (ESR1)-mutated breast cancer. In some embodiments, the breast cancer is ER+/HER2−, progesterone receptor-positive, and estrogen receptor 1 (ESR1)-mutated breast cancer. In some embodiments, the breast cancer is ERα+/HER2−, progesterone receptor-positive, and estrogen receptor 1 (ESR1)-mutated breast cancer. In some embodiments, the breast cancer is a node positive early breast cancer with a high risk of recurrence. In some embodiments, the breast cancer is an advanced or metastatic breast cancer. In some embodiments, the metastatic breast cancer is metastatic to the brain. In some embodiments, the metastatic breast cancer is naïve to CDK4/6 inhibitors (i.e., the metastatic breast cancer has not been treated with any CDK4/6 inhibitor since its metastasis). In some embodiments, the subject having metastatic breast cancer has circulating tumor DNA (ctDNA) relapse (i.e., subjects who are ctDNA-positive after definitive treatment for early breast cancer). In some embodiments, the subject is a postmenopausal woman or adult man.

In some embodiments, the breast cancer has progressed after endocrinological treatment. In some embodiments, the breast cancer progressed following at least one line of endocrinological therapy. In some embodiments, the endocrinological therapy comprises administration of one or more drugs selected from: a selective estrogen receptor degrader (SERD), an aromatase inhibitor, a selective estrogen receptor modulator (SERM), a human epidermal growth factor receptor 2 (HER2) inhibitor, a chemo therapeutic agent, a cdk4/6 inhibitor, an m-TOR inhibitor, phosphoinositide 3-kinase inhibitors (PI3K inhibitors) or rituximab.

In some embodiments, the present disclosure relates to methods for treating endometrial cancer in a subject, the methods comprising administering to the subject an effective amount of the solid pharmaceutical composition according to any of the embodiments disclosed herein. In some embodiments, the endometrial cancer is p53 wild type endometrial cancer. In some embodiments, the endometrial cancer is ER+ endometrial cancer. In some embodiments, the endometrial cancer is advanced or recurrent endometrial cancer.

The solid pharmaceutical composition is administered at least one, two, three, four, five, or six times per day, at least 1, 2, 3, 4, 5, 6, 7, 8, 9, 10, 11, 12, 13, 14, 15, 16, 17, 18, 19, 20, 21, 25, 28, 30, or 35 times per week. In some embodiments, a single administration comprises administering one, two, three, four or five tablets of the solid pharmaceutical composition. In some embodiments, the solid pharmaceutical composition is administered once per day.

An exemplary, non-limiting range for a therapeutically or prophylactically effective amount of elacestrant dihydrochloride is a dose of between 0.1 and 200 mg/kg, for example between 0.1 and 10 mg/kg, or about 20 mg/kg to about 100 mg/kg. The therapeutically or prophylactically effective amount of elacestrant dihydrochloride may be between 1 and 200 mg/kg, 10 and 200 mg/kg, 20 and 200 mg/kg, 50 and 200 mg/kg, 75 and 200 mg/kg, 100 and 200 mg/kg, 150 and 200 mg/kg, 50 and 100 mg/kg, 5 and 10 mg/kg, or 1 and 10 mg/kg. It is to be noted that dosage values may vary with the type and severity of the condition to be alleviated.

In some embodiments, the elacestrant dihydrochloride can be administered to a subject in an amount of about 10 mg/day to about 500 mg/day, about 10 mg/day to about 200 mg/day (e.g., 10, 20, 30, 40, 50, 60, 70, 80, 90, 100, 110, 120, 130, 140, 150, 160, 170, 180, 190, or 200 mg/day), 20 mg/day to about 100 mg/day, 100 mg/day to about 200 mg/day, or about 200 mg/day to about 500 mg/day (e.g., 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 mg/day), inclusive of any single or multi-dose daily administration regimen that falls within that total daily dose range. In some embodiments, the dose is from about 20 mg/day to about 100 mg/day or from about 100 mg/day to about 400 mg/day. Additionally, one of ordinary skill in the art would also know how to adjust or modify variables such as dosage, dosage schedules, and routes of administration, as appropriate, for a given subject. For instance, in some embodiments, a single administration of an elacestrant dihydrochloride composition may comprise administering 2×100 mg tablets, 3×100 mg tablets, 4×100 mg tablets, or 1×400 mg tablets.

Further, the elacestrant dihydrochloride dose may be determined by a person skilled in the art and may vary according to factors such as the disease state, age, sex, and weight of the individual, and the ability of the elacestrant dihydrochloride to elicit a desired response in the individual. The dose is also one in which toxic or detrimental effects, if any, of the elacestrant dihydrochloride are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The solid pharmaceutical composition may be formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the solid pharmaceutical composition is formulated in accordance with routine procedures as a pharmaceutical composition adapted for intravenous, subcutaneous, intramuscular, oral, intranasal, or topical administration to human beings. In some embodiments, the solid pharmaceutical composition is administered by the compound is administered by oral administration, intravenous administration, intradermal injection, intramuscular injection, or subcutaneous injection. In some embodiments, the solid pharmaceutical composition is administered by oral administration.

In some embodiments, the methods of treating breast or endometrial cancer disclosed herein further comprise the administration of an additional therapeutic agent. In some embodiments, the additional therapeutic agent is an mTOR inhibitor, a CDK4/6 inhibitor, or a threonine kinase inhibitor. In some embodiments, the mTOR inhibitor is everolimus. In some embodiments, the CDK4/6 inhibitor is palbociclib, abemaciclib, or ribociclib. In some embodiments, the threonine kinase inhibitor is capavasertib. In some embodiments, the additional therapeutic agent is fulvestrant.

An exemplary, non-limiting range for a therapeutically or prophylactically effective amount of the additional therapeutic agent is a dose of between 0.1 and 200 mg/kg, for example between 0.1 and 10 mg/kg, or about 20 mg/kg to about 100 mg/kg. The therapeutically or prophylactically effective amount of additional therapeutic agent may be between 1 and 200 mg/kg, 10 and 200 mg/kg, 20 and 200 mg/kg, 50 and 200 mg/kg, 75 and 200 mg/kg, 100 and 200 mg/kg, 150 and 200 mg/kg, 50 and 100 mg/kg, 5 and 10 mg/kg, or 1 and 10 mg/kg. It is to be noted that dosage values may vary with the type and severity of the condition to be alleviated.

In some embodiments, the additional therapeutic agent can be administered to a subject in an amount of about 10 mg/day to about 500 mg/day, about 10 mg/day to about 200 mg/day (e.g., 10, 20, 30, 40, 50, 60, 70, 80, 90, 100, 110, 120, 130, 140, 150, 160, 170, 180, 190, or 200 mg/day), 20 mg/day to about 100 mg/day, 100 mg/day to about 200 mg/day, or about 200 mg/day to about 500 mg/day (e.g., 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 mg/day), inclusive of any single or multi-dose daily administration regimen that falls within that total daily dose range. In some embodiments, the dose is from about 20 mg/day to about 100 mg/day or from about 100 mg/day to about 400 mg/day.

In some embodiments, the method further comprises administering fulvestrant to the subject. In some embodiments, the method comprises administering to the subject about 125 mg to about 750 mg, about 125 mg to about 625 mg, about 125 mg to about 500 mg, about 125 mg to about 375 mg, about 125 mg to about 250 mg, about 250 mg to about 750 mg, about 250 mg to about 625 mg, about 250 mg to about 500 mg, about 250 mg to about 375 mg, about 375 mg to about 750 mg, about 375 mg to about 625 mg, about 375 mg to about 500 mg, about 500 mg to about 750 mg, about 500 mg to about 625 mg, or about 625 mg to about 750 mg, or a corresponding amount of a pharmaceutically acceptable salt thereof. In some embodiments, the method comprises administering to the subject about 125, about 250 mg, about 375 mg, about 500 mg, about 625, or about 750 mg of fulvestrant, or a corresponding amount of a pharmaceutically acceptable salt thereof. In some embodiments, the fulvestrant is administered subcutaneously. In some embodiments, the method comprises administering fulvestrant once every two weeks for the first six weeks of the administration. In some embodiments, the method comprises administering fulvestrant once every two weeks for the first three doses of the administration, and once monthly for each dose thereafter.

### EXEMPLARY EMBODIMENTS

Embodiment I-1. A solid pharmaceutical composition comprising:
a core comprising:
elacestrant dihydrochloride, present at a concentration of 30 wt. % to 60 wt. %, relative to the total weight of the composition; and
one or more pharmaceutically acceptable excipients,
wherein the pharmaceutical composition is a tablet having a hardness of at least 5 kP.

Embodiment I-2. The composition of embodiment I-1, wherein the tablet has a hardness of at least 7 kP.

Embodiment I-3. The composition of embodiment I-1, wherein the tablet has a hardness of at least 13 kP.

Embodiment I-4. The composition of any one of embodiments I-1 to I-3, wherein the elacestrant dihydrochloride is present in an amount of 50 mg to 500 mg.

Embodiment I-5. The composition of any one of embodiments I-1 to I-4, wherein the elacestrant dihydrochloride is present in an amount of about 100 mg, about 172 mg, about 258 mg, or about 400 mg.

Embodiment I-6. The composition of any one of embodiments I-1 to I-5, wherein the elacestrant dihydrochloride is present at a concentration of 40 wt. % to 60 wt. %, relative to the total weight of the composition.

Embodiment I-7. The composition of any one of embodiments I-1 to I-6, wherein the elacestrant dihydrochloride is present at a concentration of 44 wt. % to 47 wt. %, relative to the total weight of the composition.

Embodiment I-8. The composition of any one of embodiments I-1 to I-7, wherein at least 75% of the elacestrant dihydrochloride is dissolved at 45 minutes in water at pH 4.5 or less.

Embodiment I-9. The composition of any one of embodiments I-1 to I-8, wherein at least 80% of the elacestrant dihydrochloride is dissolved after 45 minutes in water at pH 4.5 or less.

Embodiment I-10. The composition of any one of embodiments I-1 to I-9, wherein the pharmaceutical excipients comprises one or more of: microcrystalline cellulose, crospovidone, silicified microcrystalline cellulose (SMCC), colloidal silicon dioxide, or magnesium stearate.

Embodiment I-11. The composition of any one of embodiments I-1 to I-10, further comprising a film coating the core.

Embodiment I-12. The composition of embodiment I-11, wherein the film is present at a concentration of about 1 wt. % to about 5 wt. %, relative to the total weight of the composition.

Embodiment I-13. The composition of any one of embodiments I-1 to I-12, wherein the elacestrant dihydrochloride is present in granules.

Embodiment I-14. The composition of any one of embodiments I-1 to I-13, wherein the composition comprises SMCC, present at a concentration of about 10.0 wt. to about 20 wt. %, relative to the total weight of the composition.

Embodiment I-15. The composition of embodiment I-14, wherein the SMCC is present at a concentration of about 15.0 wt. % to about 20 wt. %, relative to the total weight of the composition.

Embodiment I-16. The composition of embodiment I-15, wherein the SMCC is present at a concentration of about 19.5 wt. %, relative to the total weight of the composition.

Embodiment I-17. The composition of any one of embodiments I-1 to I-16, wherein the composition is effective to achieve a plasma elacestrant free base Cₘᵢₙ at steady state of greater than or equal to 20 ng/mL in a human subject at 30 hours after a single administration.

Embodiment I-18. The composition of any one of embodiments I-1 to I-17, wherein the composition is effective to achieve a plasma elacestrant free base Cₘᵢₙ at steady state of greater than or equal to 30 ng/mL in a human subject at 30 hours after a single administration.

Embodiment I-19. A method of manufacturing a solid pharmaceutical composition, the method comprising:
compressing a mixture comprising elacestrant dihydrochloride and one or more pharmaceutically acceptable excipients to produce a tablet,
wherein:
   the mixture comprises 30 wt. % to 60 wt. % of elacestrant dihydrochloride, relative to the total weight of the mixture; and
   the compressing is performed at a pressure of at least 1 kN; and
the tablet has a hardness of at least 5 kP.

Embodiment I-20. The method of embodiment I-19, further comprising coating the tablet with a film.

Embodiment I-21. The method of embodiment I-19 or I-20, wherein the compressing performed at a pressure of at least 9 kN.

Embodiment I-22. The method of any one of embodiments I-19 to I-21, wherein the tablet has a hardness of at least 7 kP.

Embodiment I-23. The method of any one of embodiments I-19 to I-22, wherein the tablet has a hardness of at least 13 kP.

Embodiment I-24. The method of any one of embodiments I-19 to I-23, wherein the elacestrant dihydrochloride is present in an amount of 50 mg to 500 mg in the tablet.

Embodiment I-25. The method of any one of embodiments I-19 to I-24, wherein the elacestrant dihydrochloride is present in the tablet in an amount of about 100 mg, about 172 mg, about 258 mg, or about 400 mg.

Embodiment I-26. The method of any one of embodiments I-19 to I-25, wherein the elacestrant dihydrochloride is present in the mixture at a concentration of 40 wt. % to 60 wt. %, relative to the total weight of the mixture.

Embodiment I-27. The method of any one of embodiments I-19 to I-26, wherein the elacestrant dihydrochloride is present in the mixture at a concentration of 44 wt. % to 47 wt. %, relative to the total weight of the mixture.

Embodiment I-28. The method of any one of embodiments I-19 to I-27, wherein at least 75% of the elacestrant dihydrochloride in the tablet is dissolved at 45 minutes in water at pH 4.5 or less.

Embodiment I-29. The method of any one of embodiments I-19 to I-28, wherein at least 80% of the elacestrant dihydrochloride in the tablet is dissolved after 45 minutes in water at pH 4.5 or less.

Embodiment I-30. The method of any one of embodiments I-19 to I-29, wherein the mixture further comprises one or more of microcrystalline cellulose, crospovidone, silicified microcrystalline cellulose (SMCC), colloidal silicon dioxide, or magnesium stearate.

Embodiment I-31. The method of any one of embodiments I-19 to I-30, wherein the elacestrant dihydrochloride is present in granules.

Embodiment I-32. The method of any one of embodiments I-19 to I-31, wherein the mixture comprises SMCC, present at a concentration of about 10.0 wt. % to about 20.0 wt. %, relative to the total weight of the mixture.

Embodiment I-33. The method of embodiment I-32, wherein the SMCC is present at a concentration of about 15.0 wt. % to about 20.0 wt. %, relative to the total weight of mixture.

Embodiment I-34. The method of embodiment I-33, wherein the SMCC is present at a concentration of about 19.5 wt. %, relative to the total weight of the mixture.

Embodiment I-35. The method of any one of embodiments I-19 to I-34, wherein the tablet is effective to achieve a plasma elacestrant free base Cmin at steady state of greater than or equal to 20 ng/mL in a human subject at 30 hours after a single administration.

Embodiment I-36. The method of any one of embodiments I-19 to I-35, wherein the tablet is effective to achieve a plasma elacestrant free base Cmin at steady state of greater than or equal to 30 ng/ml in a human subject at 30 hours after a single administration.

Embodiment I-37. A method for treating breast cancer in a subject, the method comprising administering to the subject an effective amount of the solid pharmaceutical composition according to any one of embodiments I-1 to I-18.

Embodiment I-38. The method of embodiment I-37, wherein the breast cancer is an estrogen receptor positive breast cancer and/or a human epidermal growth factor receptor 2 (HER2)-negative breast cancer.

Embodiment I-39. The method of embodiment I-37 or I-38, wherein the breast cancer is an estrogen receptor 1 (ESR1)-mutated breast cancer.

Embodiment I-40. The method of any one of embodiments I-37 to I-39, wherein the breast cancer is ER+/HER2− and estrogen receptor 1 (ESR1)-mutated breast cancer.

Embodiment I-41. The method of any one of embodiments I-37 to I-40, wherein the breast cancer is an advanced or metastatic breast cancer.

Embodiment I-42. The method of any one of embodiments I-37 to I-41, wherein the breast cancer has progressed after at least one line of endocrinological therapy.

Embodiment I-43. The method of embodiment I-42, wherein the endocrinological therapy comprises administration of one or more drugs selected from: a selective estrogen receptor degrader (SERD), an aromatase inhibitor, a selective estrogen receptor modulator (SERM), a human epidermal growth factor receptor 2 (HER2) inhibitor, a chemo therapeutic agent, a cdk4/6 inhibitor, an m-TOR inhibitor, a phosphoinositide 3-kinase inhibitor (PI3K inhibitor), or rituximab.

Embodiment I-44. The method of any one of embodiments I-37 to I-43, wherein the solid pharmaceutical composition is administered at least once per day.

Embodiment I-45. The method of any one of embodiments I-37 to I-44, wherein the solid pharmaceutical composition is administered once per day.

Embodiment I-46. The method of any one of embodiments I-37 to I-45, wherein the solid pharmaceutical composition is administered by oral administration.

Embodiment I-47. The method of any one of embodiments I-37 to I-46, wherein the subject is a postmenopausal woman or adult man.

Enumerated Embodiment II-1. A solid pharmaceutical composition comprising:
a core comprising:
elacestrant dihydrochloride, present at a concentration of 30 wt. % to 60 wt. %, relative to the total weight of the core; and
   silicified microcrystalline cellulose (SMCC), present at a concentration of 10 wt. % to 30 wt. %, relative to the total weight of the core,
   wherein the composition is a tablet having a hardness of at least 5 kP as measured in accordance with USP <1217>.

Enumerated Embodiment II-2. The composition of Enumerated Embodiment II-1, wherein the elacestrant dihydrochloride is present at a concentration of 40 wt. % to 60 wt. %, relative to the total weight of the core.

Enumerated Embodiment II-3. The composition of Enumerated Embodiment II-1 or 2, wherein the elacestrant dihydrochloride is present at a concentration of 44 wt. % to 47 wt. %, relative to the total weight of the core.

Enumerated Embodiment II-4. The composition of any one of Enumerated Embodiments II-1 to 3, wherein the core further comprises microcrystalline cellulose, crospovidone, colloidal silicon dioxide, or magnesium stearate.

Enumerated Embodiment II-5. The composition of any one of Enumerated Embodiments II-1 to 4, wherein the SMCC is present at a concentration of about 15.0 wt. % to about 20 wt. %, relative to the total weight of the core.

Enumerated Embodiment II-6. The composition of any one of Enumerated Embodiments II-1 to 5, wherein the SMCC is present at a concentration of about 19.5 wt. %, relative to the total weight of the core.

Enumerated Embodiment II-7. The composition of any one of Enumerated Embodiments II-1 to 6, wherein the core comprises:
about 45.9 wt. % elacestrant dihydrochloride, relative to the total weight of the core;
about 26.4 wt. % microcrystalline cellulose, relative to the total weight of the core;
about 6.4 wt. % crospovidone, relative to the total weight of the core;
about 1.6 wt. % magnesium stearate, relative to the total weight of the core;
about 19.5 wt. % SMCC, relative to the total weight of the core; and
about 0.2 wt. % colloidal silicon dioxide, relative to the total weight of the core.

Enumerated Embodiment II-8. The composition of any one of Enumerated Embodiments II-1 to 7, further comprising a film coating the core.

Enumerated Embodiment II-9. The composition of Enumerated Embodiment II-8, wherein the film is present at a concentration of about 1 wt. % to about 5 wt. %, relative to the total weight of the composition.

Enumerated Embodiment II-10. A solid pharmaceutical composition comprising:
about 42 wt. % to about 47 wt. % elacestrant dihydrochloride;
about 20 wt. % to about 30 wt. % microcrystalline cellulose;
about 5 wt. % to about 10 wt. % crospovidone;
about 0.5 wt. % to about 3 wt. % magnesium stearate;
about 15 wt. % to about 25 wt. % SMCC; and
about 0.01 wt. % to about 1 wt. % colloidal silicon dioxide,
wherein the composition is a tablet.

Enumerated Embodiment II-11. The composition of any one of Enumerated Embodiments II-1 to 10, wherein the elacestrant dihydrochloride is present in an amount of 50 mg to 500 mg.

Enumerated Embodiment II-12. The composition of any one of Enumerated Embodiments II-1 to 11, wherein the elacestrant dihydrochloride is present in an amount of about 100 mg, about 172 mg, about 258 mg, or about 400 mg.

Enumerated Embodiment II-13. The composition of any one of Enumerated Embodiments II-1 to 12, wherein the elacestrant dihydrochloride is present in an amount of about 100 mg.

Enumerated Embodiment II-14. The composition of Enumerated Embodiment II-13, wherein the tablet has a hardness of about 5.4 to about 16 kP, as measured in accordance with USP <1217>.

Enumerated Embodiment II-15. The tablet of Enumerated Embodiment II-13 or 14, wherein the tablet has a hardness of about 9 kP, as measured in accordance with USP <1217>.

Enumerated Embodiment II-16. The composition of any one of Enumerated Embodiments II-1 to 12, wherein the elacestrant dihydrochloride is present in an amount of about 400 mg.

Enumerated Embodiment II-17. The composition of Enumerated Embodiment II-16, wherein the tablet has a hardness of about 12 to about 23 kP, as measured in accordance with USP <1217>.

Enumerated Embodiment II-18. The tablet of Enumerated Embodiment II-16 or 17, wherein the tablet has a hardness of about 17 kP, as measured in accordance with USP <1217>.

Enumerated Embodiment II-19. The composition of any one of Enumerated Embodiments II-1 to 18, wherein at least 85% of the elacestrant dihydrochloride is dissolved at 45 minutes in water at pH 4.5 or less, as measured in accordance with USP <711> and USP <1092> using apparatus 2.

Enumerated Embodiment II-20. The composition of Enumerated Embodiment II-19, wherein the dissolution of the elacestrant dihydrochloride is measured with the USP2 apparatus, at a stirring speed of 75 rpm, a temperature of 37° C., in 500 mL or 1000 mL of a medium of 0.01N HCl, with 5 mL samples taken through a 10-micron porous filter.

Enumerated Embodiment II-21. The composition of Enumerated Embodiment II-19, wherein the dissolution of the elacestrant dihydrochloride is measured for six single tablets with USP2 apparatus, at a stirring speed of 75 rpm from 0 to 45 minutes and a stirring speed of 250 rpm at 45 minutes onwards, a temperature of 37° C., in 500 mL or 1000 mL of a medium of 0.01N HCl, with 5 mL samples taken through a 10-micron porous filter and characterized by HPLC.

Enumerated Embodiment II-22. The composition of any one of Enumerated Embodiments II-1 to 21, wherein the elacestrant dihydrochloride is present in granules.

Enumerated Embodiment II-23. The composition of Enumerated Embodiment II-22, wherein a first portion of the SMCC is intragranular and a second portion of the SMCC is extragranular.

Enumerated Embodiment II-24. The composition of Enumerated Embodiment II-23, wherein the first portion of the SMCC is present at a concentration of about 5 wt. % to about 10 wt. %, relative to the total weight of the composition.

Enumerated Embodiment II-25. The composition of Enumerated Embodiment II-23 or 24, wherein the first portion of the SMCC is present at a concentration of about 8.0 wt. %.

Enumerated Embodiment II-26. The composition of any one of Enumerated Embodiments II-23 to 25, wherein the second portion of the SMCC is present at a concentration of about 10 wt. % to about 20 wt. %, relative to the total weight of the composition.

Enumerated Embodiment II-27. The composition of any one of Enumerated Embodiments II-23 to 26, wherein the second portion of the SMCC is present at a concentration of about 11.5 wt. %.

Enumerated Embodiment II-28. The composition of any one of Enumerated Embodiments II-1 to 27, wherein the SMCC is present in an amount of about 42.5 mg, about 170 mg, or about 184.1 mg.

Enumerated Embodiment II-29. The composition of any one of Enumerated Embodiments II-1 to 28, wherein the SMCC has an average particle size of about 125 μm, as measured by laser diffraction.

Enumerated Embodiment II-30. The composition of any one of Enumerated Embodiments II-1 to 29, wherein the SMCC has a bulk density of about 0.25 to about 0.50 g/mL.

Enumerated Embodiment II-31. The composition of any one of Enumerated Embodiments II-1 to 30, wherein the composition is effective to achieve a plasma elacestrant free base Cₘᵢₙ at steady state of greater than or equal to 20 ng/mL in a human subject at 30 hours after a single administration.

Enumerated Embodiment II-32. The composition of any one of Enumerated Embodiments II-1 to 31, wherein the composition is effective to achieve a plasma elacestrant free base Cₘᵢₙ at steady state of greater than or equal to 30 ng/ml in a human subject at 30 hours after a single administration.

Enumerated Embodiment II-33. A method of manufacturing a solid pharmaceutical composition, the method comprising:
roller compacting an intragranular blend comprising elacestrant dihydrochloride and a first portion of SMCC to provide an intragranular phase comprising granules;
mixing the intragranular phase with a second portion of SMCC to provide a mixture; and
compressing the mixture at a pressure of about 3 kN to about 40 kN to produce a tablet, wherein:
the granules comprise the elacestrant dihydrochloride and the first portion of SMCC;
the second portion of SMCC is extragranular SMCC; and
the tablet has a hardness of about 5 kP to about 23 kP as measured in accordance with USP <1217>.

Enumerated Embodiment II-34. The method of Enumerated Embodiment II-33, further comprising mixing the elacestrant dihydrochloride with the first portion of SMCC to provide the intragranular blend.

Enumerated Embodiment II-35. The method of Enumerated Embodiment II-33 or 34, further comprising coating the tablet with a film.

Enumerated Embodiment II-36. The method of any one of Enumerated Embodiments II-33 to 35, wherein the mixture further comprises microcrystalline cellulose, crospovidone, colloidal silicon dioxide, or magnesium stearate.

Enumerated Embodiment II-37. A method of manufacturing a solid pharmaceutical composition, the method comprising:
mixing elacestrant dihydrochloride, a first portion of SMCC, microcrystalline cellulose, and a first portion of crospovidone to provide an intragranular blend;
mixing the intragranular blend with a first portion of magnesium stearate to give a lubricated intragranular blend;
roller compacting the lubricated intragranular blend to provide an intragranular phase comprising granules;
mixing the intragranular phase with a second portion of SMCC, a second portion of crospovidone, and colloidal silicon dioxide to provide a compression blend;
mixing the compression blend with a second portion of magnesium stearate to provide the mixture;
compressing the mixture at a pressure of about 3 kN to about 40 kN to produce a tablet; and
coating the tablet with a film to give the composition,
wherein the granules comprise the elacestrant dihydrochloride, the first portion of SMCC, the microcrystalline cellulose, and the first portion of crospovidone, and
wherein the second portion of SMCC, the second portion of crospovidone, and the colloidal silicon dioxide are extragranular.

Enumerated Embodiment II-38. The method of any one of Enumerated Embodiments II-33 to 37, wherein the first portion of the SMCC is present in the mixture at a concentration of about 5 wt. % to about 10 wt. %, relative to the total weight of the mixture.

Enumerated Embodiment II-39. The method of any one of Enumerated Embodiments II-33 to 38, wherein the first portion of the SMCC is present in the mixture at a concentration of about 8.0 wt. %, relative to the total weight of the mixture.

Enumerated Embodiment II-40. The method of any one of Enumerated Embodiments II-33 to 39, wherein the second portion of the SMCC is present at a concentration of about 10 wt. % to about 20 wt. %, relative to the total weight of the mixture.

Enumerated Embodiment II-41. The method of any one of Enumerated Embodiments II-33 to 40, wherein the second portion of the SMCC is present at a concentration of about 11.5 wt. %, relative to the total weight of the mixture.

Enumerated Embodiment II-42. The method of any one of Enumerated Embodiments II-33 to 41, wherein the compressing is performed at a pressure of about 9 kN to about 16 kN.

Enumerated Embodiment II-43. The method of any one of Enumerated Embodiments II-33 to 42, wherein the elacestrant dihydrochloride is present in the tablet in an amount of 50 mg to 500 mg.

Enumerated Embodiment II-44. The method of any one of Enumerated Embodiments II-33 to 43, wherein the elacestrant dihydrochloride is present in the tablet in an amount of about 100 mg, about 172 mg, about 258 mg, or about 400 mg.

Enumerated Embodiment II-45. The method of any one of Enumerated Embodiments II-33 to 44, wherein the elacestrant dihydrochloride is present in the tablet in an amount of about 100 mg.

Enumerated Embodiment II-46. The method of Enumerated Embodiment II-45, wherein the tablet has a hardness of about 5.4 to about 16.0 kP, as measured in accordance with USP <1217>.

Enumerated Embodiment II-47. The method of any one of Enumerated Embodiments II-33 to 44, wherein the elacestrant dihydrochloride is present in the tablet in an amount of about 400 mg.

Enumerated Embodiment II-48. The method of Enumerated Embodiment II-47, wherein the tablet has a hardness of 12.0 to 23.0 kP, as measured in accordance with USP <1217>.

Enumerated Embodiment II-49. The method of any one of Enumerated Embodiments II-33 to 48, wherein at least 85% of the elacestrant dihydrochloride in the tablet is dissolved after 45 minutes in water at pH 4.5 or less, as measured in accordance with USP <711> and USP <1092> using apparatus 2.

Enumerated Embodiment II-50. The method of Enumerated Embodiment II-49, wherein the dissolution of the elacestrant dihydrochloride is measured with the USP2 apparatus, at a stirring speed of 75 rpm, a temperature of 37° C., in 500 mL or 1000 mL of a medium of 0.01N HCl, with 5 mL samples taken through a 10-micron porous filter.

Enumerated Embodiment II-51. The method of Enumerated Embodiment II-49, wherein the dissolution of the elacestrant dihydrochloride is measured for six single tablets with USP2 apparatus, at a stirring speed of 75 rpm from 0 to 45 minutes and a stirring speed of 250 rpm at 45 minutes onwards, a temperature of 37° C., in 500 mL or 1000 mL of a medium of 0.01N HCl, with 5 mL samples taken through a 10-micron porous filter and characterized by HPLC.

Enumerated Embodiment II-52. The method of any one of Enumerated Embodiments II-33 to 51, wherein the elacestrant dihydrochloride is present in the mixture at a concentration of 40 wt. % to 60 wt. %, relative to the total weight of the mixture.

Enumerated Embodiment II-53. The method of any one of Enumerated Embodiments II-33 to 52, wherein the elacestrant dihydrochloride is present in the mixture at a concentration of 44 wt. % to 47 wt. %, relative to the total weight of the mixture.

Enumerated Embodiment II-54. The method of any one of Enumerated Embodiments II-33 to 53, wherein the mixture comprises SMCC, present at a concentration of about 10.0 wt. % to about 20.0 wt. %, relative to the total weight of the mixture.

Enumerated Embodiment II-55. The method of any one of Enumerated Embodiments II-33 to 54, wherein the SMCC is present at a concentration of about 15.0 wt. % to about 20.0 wt. %, relative to the total weight of mixture.

Enumerated Embodiment II-56. The method of any one of Enumerated Embodiments II-33 to 55, wherein the SMCC is present at a concentration of about 19.5 wt. %, relative to the total weight of the mixture.

Enumerated Embodiment II-57. A method of manufacturing the composition of any one of Enumerated Embodiments II-1-32, the method comprising:
roller compacting an intragranular blend comprising the elacestrant dihydrochloride and a first portion of the SMCC to provide an intragranular phase comprising granules;
mixing the intragranular phase with a second portion of the SMCC to provide a mixture; and
compressing the mixture at a pressure of about 3 kN to about 40 kN to produce the tablet,
wherein the granules comprise the elacestrant dihydrochloride and the first portion of the SMCC.

Enumerated Embodiment II-58. The method of Enumerated Embodiment II-57, further comprising mixing the elacestrant dihydrochloride with the first portion of SMCC to provide the intragranular blend.

Enumerated Embodiment II-59. A method of manufacturing the composition of any one of Enumerated Embodiments II-1-32, the method comprising:
mixing the elacestrant dihydrochloride, a first portion of the SMCC, microcrystalline cellulose, and a first portion of crospovidone to provide an intragranular blend;
mixing the intragranular blend with a first portion of magnesium stearate to give a lubricated intragranular blend;
roller compacting the lubricated intragranular blend to provide an intragranular phase comprising granules;
mixing the intragranular phase with a second portion of the SMCC, a second portion of crospovidone, and colloidal silicon dioxide to provide a compression blend;
mixing the compression blend with a second portion of magnesium stearate to provide a mixture;
compressing the mixture at a pressure of about 3 kN to about 40 kN to produce the tablet; and
coating the tablet with a film to give the composition,
wherein the granules comprise the elacestrant dihydrochloride, the first portion of SMCC, the microcrystalline cellulose, and the first portion of crospovidone, and
wherein the second portion of SMCC, the second portion of crospovidone, and the colloidal silicon dioxide are extragranular.

Enumerated Embodiment II-60. A method of manufacturing a solid pharmaceutical composition in accordance with FIG. 1.

Enumerated Embodiment II-61. The composition of any one of Enumerated Embodiments II-1-32 for use as a medicament.

Enumerated Embodiment II-62. The composition of any one of Enumerated Embodiments II-1-32 for use in treating breast cancer.

Enumerated Embodiment II-63. Use of the composition of any one of Enumerated Embodiments II-1-32 for the treatment of breast cancer.

Enumerated Embodiment II-64. Use of the composition of any one of Enumerated Embodiments II-1-32 for the manufacture of a medicament for the treatment of breast cancer.

Enumerated Embodiment II-65. A method for treating breast cancer in a subject, the method comprising administering to the subject an effective amount of the composition according to any one of Enumerated Embodiments II-1 to 32.

Enumerated Embodiment II-66. The method of Enumerated Embodiment II-65, wherein the breast cancer is an estrogen receptor positive breast cancer and/or a human epidermal growth factor receptor 2 (HER2)-negative breast cancer.

Enumerated Embodiment II-67. The method of Enumerated Embodiment II-65 or 66, wherein the breast cancer is an estrogen receptor 1 (ESR1)-mutated breast cancer.

Enumerated Embodiment II-68. The method of any one of Enumerated Embodiments II-65 to 67, wherein the breast cancer is ER+/HER2− and estrogen receptor 1 (ESR1)-mutated breast cancer.

Enumerated Embodiment II-69. The method of any one of Enumerated Embodiments II-65 to 68, wherein the breast cancer is an advanced or metastatic breast cancer.

Enumerated Embodiment II-70. The method of any one of Enumerated Embodiments II-65 to 69, wherein the breast cancer has progressed after at least one line of endocrinological therapy.

Enumerated Embodiment II-71. The method of Enumerated Embodiment II-70, wherein the endocrinological therapy comprises administration of one or more drugs selected from: a selective estrogen receptor degrader (SERD), an aromatase inhibitor, a selective estrogen receptor modulator (SERM), a human epidermal growth factor receptor 2 (HER2) inhibitor, a chemo therapeutic agent, a cdk4/6 inhibitor, an m-TOR inhibitor, a phosphoinositide 3-kinase inhibitor (PI3K inhibitor), or rituximab.

Enumerated Embodiment II-72. The method of any one of Enumerated Embodiments II-65 to 71, wherein the solid pharmaceutical composition is administered at least once per day.

Enumerated Embodiment II-73. The method of any one of Enumerated Embodiments II-65 to 72, wherein the solid pharmaceutical composition is administered once per day.

Enumerated Embodiment II-74. The method of any one of Enumerated Embodiments II-65 to 73, wherein the solid pharmaceutical composition is administered by oral administration.

Enumerated Embodiment II-75. The method of any one of Enumerated Embodiments II-65 to 74, wherein the subject is a postmenopausal woman or adult man.

Enumerated Embodiment II-76. The method of any one of Enumerated Embodiments II-65 to 75, further comprising administering to the subject about 125 to about 750 mg of fulvestrant, or a corresponding amount of a pharmaceutically acceptable salt thereof.

Enumerated Embodiment II-77. The method of Enumerated Embodiment II-76 wherein the fulvestrant is administered subcutaneously.

Enumerated Embodiment II-78. The method of Enumerated Embodiment II-76 or 77 wherein the fulvestrant is administered once every two weeks for the first six weeks of the administration.

Enumerated Embodiment II-79. The method of Enumerated Embodiment II-76 or 77, wherein the fulvestrant is administered once every two weeks for the first three doses of the administration, and once monthly for each dose thereafter.

Enumerated Embodiment III-1. A solid pharmaceutical composition comprising:
a core comprising:
elacestrant dihydrochloride, present at a concentration of about 30 wt. % to about 60 wt. %, relative to the total weight of the core; and
silicified microcrystalline cellulose (SMCC) or a blend comprising microcrystalline cellulose and colloidal silicon dioxide, wherein the SMCC or the blend is present at a concentration of about 5 wt. % to about 25 wt. %, relative to the total weight of the core,
wherein the composition is a tablet having a hardness of at least about 5 kP as measured in accordance with USP <1217>.

Enumerated Embodiment III-2. The composition of Enumerated Embodiment III-1, wherein the elacestrant dihydrochloride is present at a concentration of about 40 wt. % to about 60 wt. %, relative to the total weight of the core.

Enumerated Embodiment III-3. The composition of Enumerated Embodiment III-1 or 2, wherein the elacestrant dihydrochloride is present at a concentration of about 44 wt. % to about 47 wt. %, relative to the total weight of the core.

Enumerated Embodiment III-4. The composition of any one of Enumerated Embodiments III-1 to 3, wherein the core further comprises one or more of a binder, a disintegrant, a glidant, or a lubricant.

Enumerated Embodiment III-5. The composition of Enumerated Embodiment III-4, wherein the binder comprises one or more of gum arabic, gelatin, sodium alginate, pullulan, starch, pregelatinized starch, gum tragacanth, carboxymethylcellulose sodium, dextrin, hydroxyethyl cellulose, hydroxypropyl cellulose, hypromellose, maltodextrin, methylcellulose, polyethylene glycol, polyvinyl alcohols, povidone, copovidone, or microcrystalline cellulose.

Enumerated Embodiment III-6. The composition of Enumerated Embodiment III-4 or 5, wherein the disintegrant comprises one or more of starch, microcrystalline cellulose, sodium alginate, croscarmellose sodium, crospovidone, sodium starch glycolate, or partially pregelatinized starch.

Enumerated Embodiment III-7. The composition of any one of Enumerated Embodiments III-4 to 6, wherein the glidant comprises one or more of colloidal silicon dioxide, talc, starch, ascorbyl palmitate, calcium palmitate, or magnesium stearate.

Enumerated Embodiment III-8. The composition of any one of Enumerated Embodiments III-4 to 7, wherein the lubricant comprises one or more of magnesium stearate, stearic acid, vegetable stearin, sodium stearyl fumarate, glyceryl di-behenate, talc, silica, polyethylene glycol, or sodium lauryl sulfate.

Enumerated Embodiment III-9. The composition of any one of Enumerated Embodiments III-1 to 3, wherein the core further comprises microcrystalline cellulose, crospovidone, colloidal silicon dioxide, or magnesium stearate.

Enumerated Embodiment III-10. The composition of any one of Enumerated Embodiments III-1 to 9, wherein the SMCC or the blend is present at a concentration of about 15.0 wt. % to about 20 wt. %, relative to the total weight of the core.

Enumerated Embodiment III-11. The composition of any one of Enumerated Embodiments III-1 to 10, wherein the SMCC or the blend is present at a concentration of about 19.5 wt. %, relative to the total weight of the core.

Enumerated Embodiment III-12. The composition of any one of Enumerated Embodiments III-1 to 11, wherein the core comprises:
about 45.9 wt. % elacestrant dihydrochloride, relative to the total weight of the core;
about 26.4 wt. % microcrystalline cellulose, relative to the total weight of the core;
about 6.4 wt. % crospovidone, relative to the total weight of the core;
about 1.6 wt. % magnesium stearate, relative to the total weight of the core;
about 19.5 wt. % SMCC, relative to the total weight of the core; and
about 0.2 wt. % colloidal silicon dioxide, relative to the total weight of the core.

Enumerated Embodiment III-13. The composition of any one of Enumerated Embodiments III-1 to 12, further comprising a film coating the core.

Enumerated Embodiment III-14. The composition of Enumerated Embodiment III-13, wherein the film is present at a concentration of about 1 wt. % to about 5 wt. %, relative to the total weight of the composition.

Enumerated Embodiment III-15. The composition of any one of Enumerated Embodiments III-1 to 14, wherein the elacestrant dihydrochloride is present in granules.

Enumerated Embodiment III-16. The composition of Enumerated Embodiment III-15, wherein a first portion of the SMCC or the blend is intragranular and a second portion of the SMCC or the blend is extragranular.

Enumerated Embodiment III-17. The composition of Enumerated Embodiment III-16, wherein the first portion of the SMCC or the blend is present at a concentration of about 5 wt. % to about 10 wt. %, relative to the total weight of the core.

Enumerated Embodiment III-18. The composition of Enumerated Embodiment III-16 or 17, wherein the first portion of the SMCC or the blend is present at a concentration of about 8.0 wt. %, relative to the total weight of the core.

Enumerated Embodiment III-19. The composition of any one of Enumerated Embodiments III-16 to 18, wherein the second portion of the SMCC or the blend is present at a concentration of about 10 wt. % to about 20 wt. %, relative to the total weight of the core.

Enumerated Embodiment III-20. The composition of any one of Enumerated Embodiments III-16 to 19, wherein the second portion of the SMCC or the blend is present at a concentration of about 11.5 wt. %, relative to the total weight of the core.

Enumerated Embodiment III-21. A solid pharmaceutical composition comprising:
about 42 wt. % to about 47 wt. % elacestrant dihydrochloride;
about 20 wt. % to about 30 wt. % microcrystalline cellulose;
about 5 wt. % to about 10 wt. % crospovidone;
about 0.2 wt. % to about 3 wt. % magnesium stearate;
about 15 wt. % to about 25 wt. % SMCC; and
about 0.01 wt. % to about 1 wt. % colloidal silicon dioxide,
wherein the composition is a tablet.

Enumerated Embodiment III-22. The composition of Enumerated Embodiment III-21, wherein the elacestrant dihydrochloride is present in granules.

Enumerated Embodiment III-23. The composition of Enumerated Embodiment III-22, wherein a first portion of the SMCC is intragranular and a second portion of the SMCC is extragranular.

Enumerated Embodiment III-24. The composition of Enumerated Embodiment III-23, wherein the first portion of the SMCC is present at a concentration of about 5 wt. % to about 10 wt. %.

Enumerated Embodiment III-25. The composition of Enumerated Embodiment III-23 or 24, wherein the first portion of the SMCC is present at a concentration of about 8.0 wt. %.

Enumerated Embodiment III-26. The composition of any one of Enumerated Embodiments III-23 to 25, wherein the second portion of the SMCC is present at a concentration of about 10 wt. % to about 20 wt. %.

Enumerated Embodiment III-27. The composition of any one of Enumerated Embodiments III-23 to 26, wherein the second portion of the SMCC is present at a concentration of about 11.5 wt. %.

Enumerated Embodiment III-28. The composition of any one of Enumerated Embodiments III-1 to 27, wherein the elacestrant dihydrochloride is present in an amount of about 100 mg.

Enumerated Embodiment III-29. The composition of Enumerated Embodiment III-28, wherein the tablet has a hardness of about 5.4 to about 16 kP, as measured in accordance with USP <1217>.

Enumerated Embodiment III-30. The tablet of Enumerated Embodiment III-28 or 29, wherein the tablet has a hardness of about 9 kP, as measured in accordance with USP <1217>.

Enumerated Embodiment III-31. The composition of any one of Enumerated Embodiments III-1 to 27, wherein the elacestrant dihydrochloride is present in an amount of about 400 mg.

Enumerated Embodiment III-32. The composition of Enumerated Embodiment III-31, wherein the tablet has a hardness of about 12 to about 23 kP, as measured in accordance with USP <1217>.

Enumerated Embodiment III-33. The tablet of Enumerated Embodiment III-31 or 32, wherein the tablet has a hardness of about 17 kP, as measured in accordance with USP <1217>.

Enumerated Embodiment III-34. The composition of any one of Enumerated Embodiments III-1 to 33, wherein at least 85% of the elacestrant dihydrochloride is dissolved at 45 minutes in a medium of 0.01 N HCl, as measured in accordance with USP <711> and USP <1092> using apparatus 2.

Enumerated Embodiment III-35. The composition of Enumerated Embodiment III-34, wherein the dissolution of the elacestrant dihydrochloride is measured with the USP2 apparatus, at a stirring speed of 75 rpm, a temperature of 37° C., in 500 mL or 1000 mL of the medium, with 5 mL samples taken through a 10-micron porous filter.

Enumerated Embodiment III-36. The composition of any one of Enumerated Embodiments III-1 to 11, 13 to 20, and 28 to 35, wherein the core comprises SMCC.

Enumerated Embodiment III-37. The composition of any one of Enumerated Embodiments III-1 to 36, wherein the SMCC is present in an amount of about 42.5 mg, about 170 mg, or about 184.1 mg.

Enumerated Embodiment III-38. The composition of any one of Enumerated Embodiments III-1 to 11, 13 to 20, and 28 to 35, wherein the core comprises the blend.

Enumerated Embodiment III-39. The composition of Enumerated Embodiment III-38 wherein the blend comprises granules comprising the microcrystalline cellulose and the colloidal silicon dioxide.

Enumerated Embodiment III-40. A method of manufacturing a solid pharmaceutical composition, the method comprising:
granulating an intragranular blend comprising elacestrant dihydrochloride and a first portion of a filler to provide an intragranular phase comprising granules;
mixing the intragranular phase with a second portion of the filler to provide a mixture; and
compressing the mixture at a pressure of about 3 kN to about 35 kN to produce a tablet, wherein:
the granules comprise the elacestrant dihydrochloride and the first portion of the filler;
the second portion of the filler is extragranular;
the filler is SMCC or a blend comprising microcrystalline cellulose and colloidal silicon dioxide; and
the tablet has a hardness of about 5 kP to about 23 kP as measured in accordance with USP <1217>

Enumerated Embodiment III-41. The method of Enumerated Embodiment III-40, wherein the granulating comprises roller compacting the intragranular blend to provide the intragranular phase.

Enumerated Embodiment III-42. The method of Enumerated Embodiment III-40 or 41, further comprising mixing the elacestrant dihydrochloride with the first portion of the filler to provide the intragranular blend.

Enumerated Embodiment III-43. The method of any one of Enumerated Embodiments III-40 to 42, further comprising coating the tablet with a film.

Enumerated Embodiment III-44. The method of any one of Enumerated Embodiments III-40 to 43, further comprising combining microcrystalline cellulose and colloidal silicon dioxide, prior to the compacting of the intragranular blend, to provide the filler.

Enumerated Embodiment III-45. The method of Enumerated Embodiment III-44, wherein the filler is the SMCC.

Enumerated Embodiment III-46. The method of Enumerated Embodiment III-44 or 45, wherein the combining comprises:
adding the colloidal silicon dioxide to an aqueous slurry of the microcrystalline cellulose to provide a mixed slurry; and
spray drying the mixed slurry to provide the SMCC.

Enumerated Embodiment III-47. The method of Enumerated Embodiment III-44 wherein the filler is the blend.

Enumerated Embodiment III-48. The method of Enumerated Embodiment III-44 or 47 wherein the blend comprises granules comprising the microcrystalline cellulose and the colloidal silicon dioxide.

Enumerated Embodiment III-49. The method of any one of Enumerated Embodiments III-40 to 48, wherein the mixture further comprises one or more of a binder, a disintegrant, a glidant, or a lubricant.

Enumerated Embodiment III-50. A method of manufacturing a solid pharmaceutical composition, the method comprising,
mixing elacestrant dihydrochloride, a first portion of SMCC, microcrystalline cellulose, and a first portion of crospovidone to provide an intragranular blend;
mixing the intragranular blend with a first portion of magnesium stearate to give a lubricated intragranular blend;
roller compacting the lubricated intragranular blend to provide an intragranular phase comprising granules;
mixing the intragranular phase with a second portion of SMCC, a second portion of crospovidone, and colloidal silicon dioxide to provide a compression blend;
mixing the compression blend with a second portion of magnesium stearate to provide the mixture;
compressing the mixture at a pressure of about 3 kN to about 35 kN to produce a tablet; and
coating the tablet with a film to give the composition,
wherein the granules comprise the elacestrant dihydrochloride, the first portion of SMCC, the microcrystalline cellulose, and the first portion of crospovidone, and
wherein the second portion of SMCC, the second portion of crospovidone, and the colloidal silicon dioxide are extragranular.

Enumerated Embodiment III-51. The method of any one of Enumerated Embodiments III-40 to 46, 49, and 50, wherein the first portion of the SMCC is present in the mixture at a concentration of about 5 wt. % to about 10 wt. %, relative to the total weight of the mixture.

Enumerated Embodiment III-52. The method of any one of Enumerated Embodiments III-40 to 46 and 49 to 51, wherein the first portion of the SMCC is present in the mixture at a concentration of about 8.0 wt. %, relative to the total weight of the mixture.

Enumerated Embodiment III-53. The method of any one of Enumerated Embodiments III-40 to 46 and 49 to 52, wherein the second portion of the SMCC is present at a concentration of about 10 wt. % to about 20 wt. %, relative to the total weight of the mixture.

Enumerated Embodiment III-54. The method of any one of Enumerated Embodiments III-40 to 46 and 49 to 53, wherein the second portion of the SMCC is present at a concentration of about 11.5 wt. %, relative to the total weight of the mixture.

Enumerated Embodiment III-55. The method of any one of Enumerated Embodiments III-40 to 54, wherein the compressing is performed at a pressure of about 10 kN to about 15 kN.

Enumerated Embodiment III-56. The method of any one of Enumerated Embodiments III-40 to 55, wherein the elacestrant dihydrochloride is present in the tablet in an amount of about 100 mg.

Enumerated Embodiment III-57. The method of Enumerated Embodiment III-56, wherein the tablet has a hardness of about 5.4 to about 16.0 kP, as measured in accordance with USP <1217>.

Enumerated Embodiment III-58. The method of any one of Enumerated Embodiments III-40 to 55, wherein the elacestrant dihydrochloride is present in the tablet in an amount of about 400 mg.

Enumerated Embodiment III-59. The method of Enumerated Embodiment III-58, wherein the tablet has a hardness of 12.0 to 23.0 kP, as measured in accordance with USP <1217>.

Enumerated Embodiment III-60. The method of any one of Enumerated Embodiments III-40 to 59, wherein at least 85% of the elacestrant dihydrochloride in the tablet is dissolved after 45 minutes in water at pH 4.5 or less, as measured in accordance with USP <711> and USP <1092> using apparatus 2.

Enumerated Embodiment III-61. The method of Enumerated Embodiment III-60, wherein the dissolution of the elacestrant dihydrochloride is measured with the USP2 apparatus, at a stirring speed of 75 rpm, a temperature of 37° C., in 500 mL or 1000 mL of a medium of 0.01N HCl, with 5 mL samples taken through a 10-micron porous filter.

Enumerated Embodiment III-62. The method of any one of Enumerated Embodiments III-40 to 61, wherein the elacestrant dihydrochloride is present in the mixture at a concentration of 40 wt. % to 60 wt. %, relative to the total weight of the mixture.

Enumerated Embodiment III-63. The method of any one of Enumerated Embodiments III-40 to 62, wherein the elacestrant dihydrochloride is present in the mixture at a concentration of 44 wt. % to 47 wt. %, relative to the total weight of the mixture.

Enumerated Embodiment III-64. The method of any one of Enumerated Embodiments III-40 to 46 and 49 to 63, wherein the mixture comprises SMCC, present at a concentration of about 10.0 wt. % to about 20.0 wt. %, relative to the total weight of the mixture.

Enumerated Embodiment III-65. The method of any one of Enumerated Embodiments III-40 to 46 and 49 to 64, wherein the SMCC is present at a concentration of about 15.0 wt. % to about 20.0 wt. %, relative to the total weight of mixture.

Enumerated Embodiment III-66. The method of any one of Enumerated Embodiments III-40 to 46 and 49 to 65, wherein the SMCC is present at a concentration of about 19.5 wt. %, relative to the total weight of the mixture.

Enumerated Embodiment III-67. A method of manufacturing the composition of any one of Enumerated Embodiments III-1 to 37, the method comprising:
roller compacting an intragranular blend comprising the elacestrant dihydrochloride and a first portion of the SMCC to provide an intragranular phase comprising granules;
mixing the intragranular phase with a second portion of the SMCC to provide a mixture; and
compressing the mixture at a pressure of about 3 kN to about 35 kN to produce the tablet,
wherein the granules comprise the elacestrant dihydrochloride and the first portion of the SMCC.

Enumerated Embodiment III-68. The method of Enumerated Embodiment III-67, further comprising mixing the elacestrant dihydrochloride with the first portion of SMCC to provide the intragranular blend.

Enumerated Embodiment III-69. A method of manufacturing the composition of any one of Enumerated Embodiments III-1 to 37, the method comprising,
mixing the elacestrant dihydrochloride, a first portion of the SMCC, microcrystalline cellulose, and a first portion of crospovidone to provide an intragranular blend;
mixing the intragranular blend with a first portion of magnesium stearate to give a lubricated intragranular blend;
roller compacting the lubricated intragranular blend to provide an intragranular phase comprising granules;
mixing the intragranular phase with a second portion of the SMCC, a second portion of crospovidone, and colloidal silicon dioxide to provide a compression blend;
mixing the compression blend with a second portion of magnesium stearate to provide a mixture;
compressing the mixture at a pressure of about 3 kN to about 35 kN to produce the tablet; and
coating the tablet with a film to give the composition,
wherein the granules comprise the elacestrant dihydrochloride, the first portion of SMCC, the microcrystalline cellulose, and the first portion of crospovidone, and
wherein the second portion of SMCC, the second portion of crospovidone, and the colloidal silicon dioxide are extragranular.

Enumerated Embodiment III-70. A method of manufacturing a solid pharmaceutical composition in accordance with FIG. 1.

Enumerated Embodiment III-71. A method of manufacturing a solid pharmaceutical composition in accordance with FIG. 2.

Enumerated Embodiment III-72. The composition prepared by any one of Enumerated Embodiments III-40 to 71.

Enumerated Embodiment III-73. The composition of any one of Enumerated Embodiments III-1 to 39 and 72 for use as a medicament.

Enumerated Embodiment III-74. The composition of any one of Enumerated Embodiments III-1 to 39 and 72 for use in treating breast cancer.

Enumerated Embodiment III-75. Use of the composition of any one of Enumerated Embodiments III-1 to 39 and 72 for the treatment of breast cancer.

Enumerated Embodiment III-76. Use of the composition of any one of Enumerated Embodiments III-1 to 39 and 72 for the manufacture of a medicament for the treatment of breast cancer.

Enumerated Embodiment III-77. A method for treating breast cancer in a subject, the method comprising administering to the subject an effective amount of the composition according to any one of Enumerated Embodiments III-1 to 39 and 72.

Enumerated Embodiment III-78. The method of Enumerated Embodiment III-77, wherein the breast cancer is an estrogen receptor positive (ER+) breast cancer and/or a human epidermal growth factor receptor 2 (HER2)-negative breast cancer.

Enumerated Embodiment III-79. The method of Enumerated Embodiment III-77 or 78, wherein the breast cancer is an estrogen receptor 1 (ESR1)-mutated breast cancer.

Enumerated Embodiment III-80. The method of any one of Enumerated Embodiments III-77 to 79, wherein the breast cancer is ER+/HER2− and estrogen receptor 1 (ESR1)-mutated breast cancer.

Enumerated Embodiment III-81. The method of any one of Enumerated Embodiments III-77 to 80, wherein the breast cancer is ER+/HER2−, progesterone receptor-positive, and estrogen receptor 1 (ESR1)-mutated breast cancer.

Enumerated Embodiment III-82. The method of any one of Enumerated Embodiments III-77 to 81, wherein the breast cancer is an estrogen receptor alpha positive (ERα+) breast cancer

Enumerated Embodiment III-83. The method of any one of Enumerated Embodiments III-77 to 82, wherein the breast cancer is a node positive early breast cancer with a high risk of recurrence.

Enumerated Embodiment III-84. The method of any one of Enumerated Embodiments III-77 to 83, wherein the breast cancer is an advanced or metastatic breast cancer.

Enumerated Embodiment III-85. The method of Enumerated Embodiment III-84, wherein the metastatic breast cancer is metastatic to the brain.

Enumerated Embodiment III-86. The method of Enumerated Embodiment III-84 or 85, wherein the metastatic breast cancer is naïve to CDK4/6 inhibitors.

Enumerated Embodiment III-87. The method of any one of Enumerated Embodiments III-77 to 86, wherein the subject has ctDNA relapse.

Enumerated Embodiment III-88. The method of any one of Enumerated Embodiments III-77 to 87, wherein the breast cancer has progressed after at least one line of endocrinological therapy.

Enumerated Embodiment III-89. The method of Enumerated Embodiment III-88, wherein the endocrinological therapy comprises administration of one or more drugs selected from: a selective estrogen receptor degrader (SERD), an aromatase inhibitor, a selective estrogen receptor modulator (SERM), a human epidermal growth factor receptor 2 (HER2) inhibitor, a chemo therapeutic agent, a cdk4/6 inhibitor, an m-TOR inhibitor, a phosphoinositide 3-kinase inhibitor (PI3K inhibitor), or rituximab.

Enumerated Embodiment III-90. A method for treating endometrial cancer in a subject, the method comprising administering to the subject an effective amount of the composition according to any one of Enumerated Embodiments III-1 to 39 and 72.

Enumerated Embodiment III-91. The method of Enumerated Embodiment III-90, wherein the endometrial cancer is p53 wild type ER+advanced or recurrent endometrial cancer.

Enumerated Embodiment III-92. The method of any one of Enumerated Embodiments III-77 to 91, wherein the solid pharmaceutical composition is administered at least once per day.

Enumerated Embodiment III-93. The method of any one of Enumerated Embodiments III-77 to 92, wherein the solid pharmaceutical composition is administered once per day.

Enumerated Embodiment III-94. The method of any one of Enumerated Embodiments III-77 to 93, wherein the solid pharmaceutical composition is administered by oral administration.

Enumerated Embodiment III-95. The method of any one of Enumerated Embodiments III-77 to 94, wherein the subject is a postmenopausal woman or adult man.

Enumerated Embodiment III-96. The method of any one of Enumerated Embodiments III-77 to 95, further comprising administering to the subject an additional therapeutic agent.

Enumerated Embodiment III-97. The method of Enumerated Embodiment III-96, wherein the additional therapeutic agent is an mTOR inhibitor, a CDK4/6 inhibitor, or a threonine kinase inhibitor.

Enumerated Embodiment III-98. The method of Enumerated Embodiment III-97, wherein the mTOR inhibitor is everolimus.

Enumerated Embodiment III-99. The method of Enumerated Embodiment III-97 or 98, wherein the CDK4/6 inhibitor is palbociclib, abemaciclib, or ribociclib.

Enumerated Embodiment III-100. The method of any one of Enumerated Embodiments III-97 to 99, wherein the threonine kinase inhibitor is capavasertib.

### EXAMPLES

### Example 1. Compressibility of Elacestrant Dihydrochloride

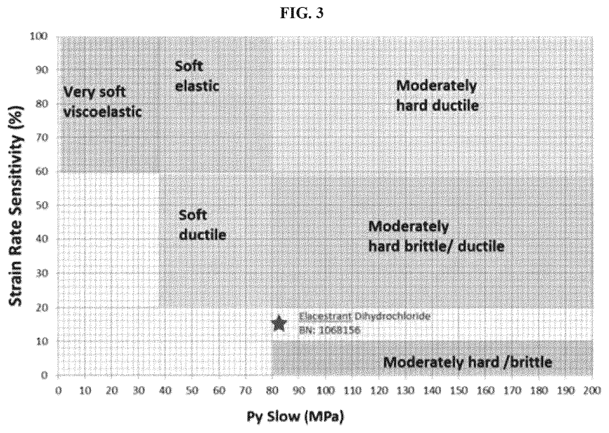

To investigate the behavior of elacestrant dihydrochloride under mechanical stress, a compressibility study was performed. The results are shown in FIG. 3. Elacestrant dihydrochloride showed a significant strain rate sensitivity at 15.5%. Thus, deformation was expected to be affected by speed. Crushing strength was rather low at slow speed and decreased as speed increased due to elastic recovery. High ejection forces were observed at slow speed which could lead to sticking to the tablet tooling during compression runs. The insights and recommendations from this work led to careful selection of excipients to keep tablet strength high for tablets with drug loads above 25%.

### Example 2. Effect of Disintegrant Level on Dissolution

To improve tablet disintegration, two development formulations were prepared with different disintegrant levels, and the effect of disintegrant concentration and tablet hardness levels on dissolution were investigated. The hardness levels provided herein were all measured in accordance with USP <1217>. In general, the increase of the disintegrant level did not improve tablet disintegration time, as shown in Table 1. Thus, the “low disintegrant” level was selected for further development.

TABLE 1
Effect of Disintegrant Level and Tablet Hardness on
Disintegration Time of Elacestrant Tablets (400 mg)

| | | | | Average | |
|---|---|---|---|---|---|
| | Disintegrant | Tablet | | Disintegration | |
| Formulation | Level | Hardness | | Time | |
| “Low Disintegrant” | 2.4 wt. % | 12.5 | kP | ~11.1 | min. |
| | | ~19 | kP | ~18.4 | min. |
| “High Disintegrant” | 5.6 wt. % | 12.5 | kP | ~8.4 | min. |
| | | ~19 | kP | ~18.4 | min. |

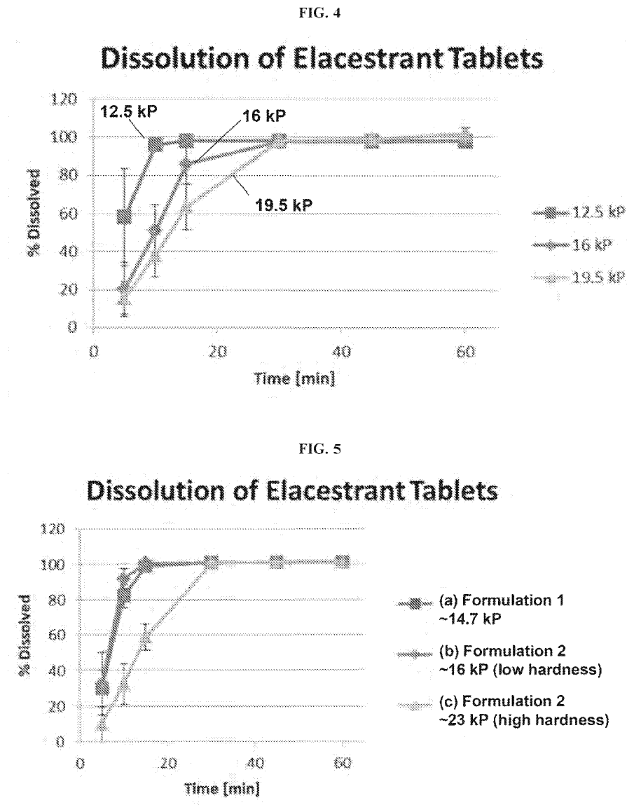

To investigate relationship between tablet hardness and dissolution profile, dissolution was compared for tablets having the “low disintegrant” formulation shown in Table 2, at different hardness values. The dissolution profiles of the formulations disclosed herein were measured for six single tablets with USP2 apparatus, at a stirring speed of 75 rpm from 0 to 45 minutes and a stirring speed of 250 rpm at 45 minutes onwards, a temperature of 37° C., in 500 mL (for 100 mg tablets) or 1000 mL (for 400 mg tablets) of a 0.01 N HCl medium (the QC medium), with 5 mL samples, and no medium replacement, taken through a 10-micron porous filter and characterized by HPLC. Referring to FIG. 4, the data shows a clear relationship between tablet hardness and disintegration time for the “low disintegrant” formulation.

TABLE 2
“Low-Disintegrant” Formulation

| Component | mg/tablet | wt. % |
|---|---|---|
| Elacestrant Dihydrochloride | 400 | 45.23 |
| Microcrystalline Cellulose | 226.86 | 26.65 |
| Crospovidone | 56.61 | 6.40 |
| Magnesium Stearate | 14.15 | 1.60 |
| SMCC | 184.13 | 20.82 |
| Colloidal Silicon Dioxide | 2.65 | 0.30 |
| Total Tablet Core Weight | 884.4 mg | 100.0 |

### Example 3. Evaluation of Effect of Tablet Hardness on Oral Bioavailability of Elacestrant Tablets

Due to the strong correlation of tablet hardness and dissolution, the impact of tablet hardness on bioavailability was further investigated using different formulations, shown in Table 3 below. Two prototypes (Formulation 2a and 2b, Table 4) with low and high hardness, respectively, were manufactured and compared to the Phase 3 formulation (Formulation 1). Formulations 2a and 2b were prepared by the method depicted by FIG. 1. The respective dissolution profiles are shown in FIG. 5. While the dissolution of the low hardness prototype (Formulation 2a) was similar to the Phase 3 comparator (Formulation 1), the high hardness prototype (Formulation 2b) had a significantly slower dissolution. Despite their different dissolution behavior, both prototypes (Formulas 2a and 2b) were found to be bioequivalent to the Phase 3 comparator (Formula 1). Formulas 2a and 2b were placed on ICH stability to confirm the stability of the product. Since the Formula 2 was a simplified version of the Phase 3 formulation (Formula 1), with fewer excipients (Table 3), a comparable stability profile was expected. This was confirmed in stability studies at long term and accelerated conditions, where the high and low hardness prototypes showed excellent stability comparable to the Phase 3 formulation. In addition to the real-time stability data, an in-silico stability assessment was performed via an Accelerated Stability Assessment Program (ASAP) that confirmed suitable stability of the pilot PK prototype drug product.

TABLE 3
Exemplary Elacestrant Dihydrochloride Tablet Formulations

| | | Formulation 1 | | | Formulation 2 | | |
|---|---|---|---|---|---|---|---|
| Component | Function | wt. % | 100 mg | 400 mg | wt. % | 100 mg | 400 mg |
| Elacestrant Dihydrochloride | API | 45.23 | 100.0 | 400.0 | 45.9 | 100.0 | 400.0 |
| Microcrystalline Cellulose | Binder | 14.93 | 33.0 | 132.0 | 26.4 | 57.5 | 230 |
| Croscarmellose Sodium | Disintegrant | 5.52 | 10.0 | 40.0 | — | — | — |
| Crospovidone | Disintegrant | 5.52 | 10.0 | 40.0 | 6.4 | 14.0 | 56 |
| Sodium Starch Glycolate | Disintegrant | 5.52 | 10.0 | 40.0 | — | — | — |
| SMCC¹ | Filler | — | — | — | 19.5 | 42.5 | 170 |
| Colloidal Silicon Dioxide | Glidant | 1.35 | 3.0 | 84.0 | 0.2 | 0.5 | 2.0 |
| Magnesium Stearate | Lubricant | 1.40 | 3.1 | 12.4 | 1.6 | 3.5 | 14 |
| Mannitol | Filler | 18.82 | 41.6 | 166.4 | — | — | — |
| Partially Pregelatinized Starch | Disintegrant | 4.70 | 10.4 | 41.6 | — | — | — |
| Core Tablet Weight, mg | | 100.0 | 221.1 | 884.4 | 100.0 | 218.0 | 872.0 |
| OPADRY ® II 85F18422 White² | Film Coating | 3.0 | 6.63 | 26.5 | 3.0 | 6.54 | 26.16 |
| Coated Tablet Weight, mg | | 103.0 | 227.7 | 910.9 | 103.0 | 224.5 | 898.2 |

¹SMCC = silicified microcrystalline cellulose, composed of microcrystalline cellulose and colloidal silica. For these examples, a commercial product (PROSOLV ® SMCC 90 from JRS Pharma) was used.
²Contains talc, polyethylene glycol, polyvinyl alcohol, and titanium dioxide.

TABLE 4
Hardness of Prototype Formulations 2a and 2b and Formulation 1

| Formulation | Hardness | |
|---|---|---|
| Formulation 2a (Low Hardness) | ~16 | kP |
| Formulation 2b (High Hardness) | ~23 | kP |
| Formulation 1 (Phase 3 Comparator) | ~14.7 | kP |

### Example 4. Further Studies of Effect of Hardness on Dissolution Profile

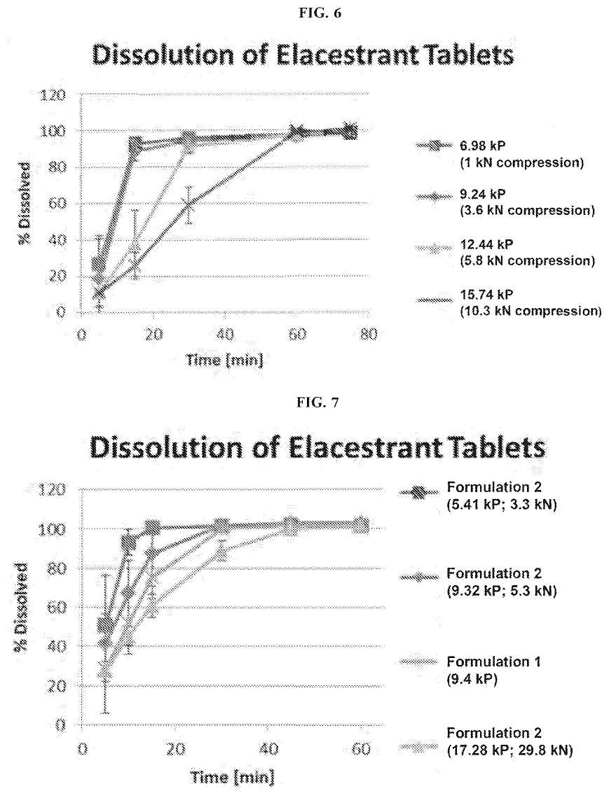

Further studies were conducted to assess the effect of tablet hardness on dissolution profile for Formulation 2. FIG. 6 shows dissolution of 100 mg tablets of Formulation 2 having different hardness values. The resulting tablets exhibited the same correlation of tablet hardness and dissolution as observed previously (see FIGS. 3-4).

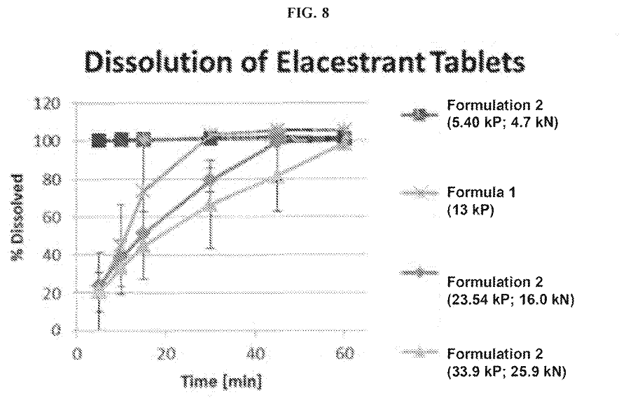

Additional dissolution studies were performed for Formulation 2 with increasing compression force to identify the target tablet hardness to produce tablets with similar dissolution profiles as the Phase 3 comparator (Formulation 1). For the 100 mg tablets, the quantity of elacestrant dihydrochloride released decreased (≥85% elacestrant dihydrochloride dissolution was shifted from 10 min to 30 min) with increasing (3.3 kN to 29.8 kN) compression force (FIG. 7). The same behavior was observed for the 400 mg tablets (FIG. 8). The quantity of elacestrant dihydrochloride released decreased (≥85% elacestrant dihydrochloride dissolution was shifted from 5 min to 60 min) with increasing (4.7 kN to 25.9 kN) compression force and increasing hardness.

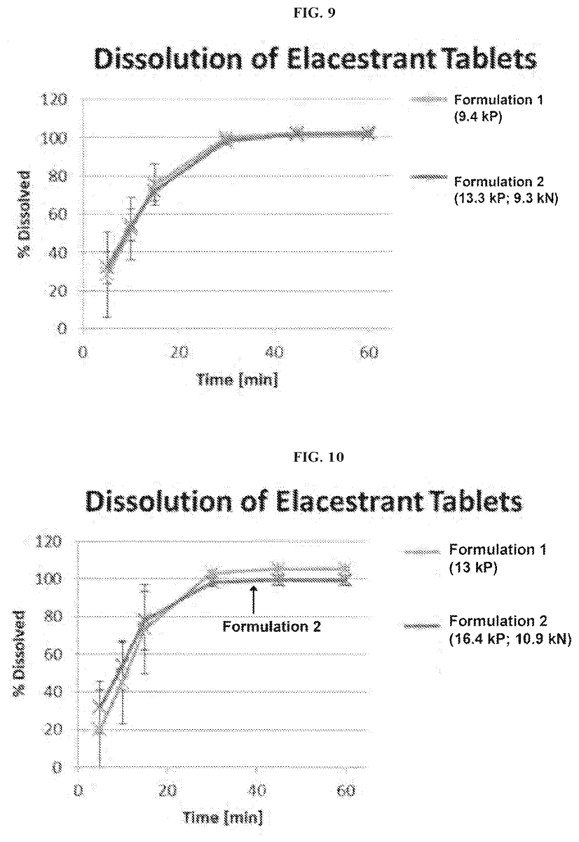

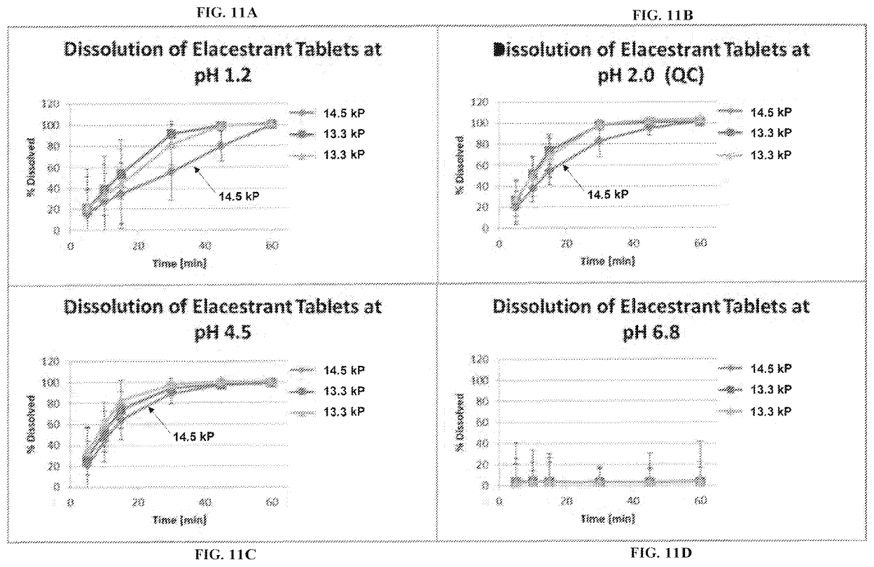

Based on the data, compression force and hardness were identified for both 100 mg and 400 mg tablets (Formulation 2) to yield dissolution profiles comparable to the Phase 3 comparator (Formulation 1). FIG. 9 and FIG. 10 show dissolution profiles for Formulation 2, 100 mg and 400 mg tablets, respectively, at the target compression and hardness values, compared to the Phase 3 comparator (Formulation 1).

### Example 5. Comparison of Dissolution Profiles for Formulation 2 at Target Hardness Versus Phase 3 Comparator (Formulation 1)

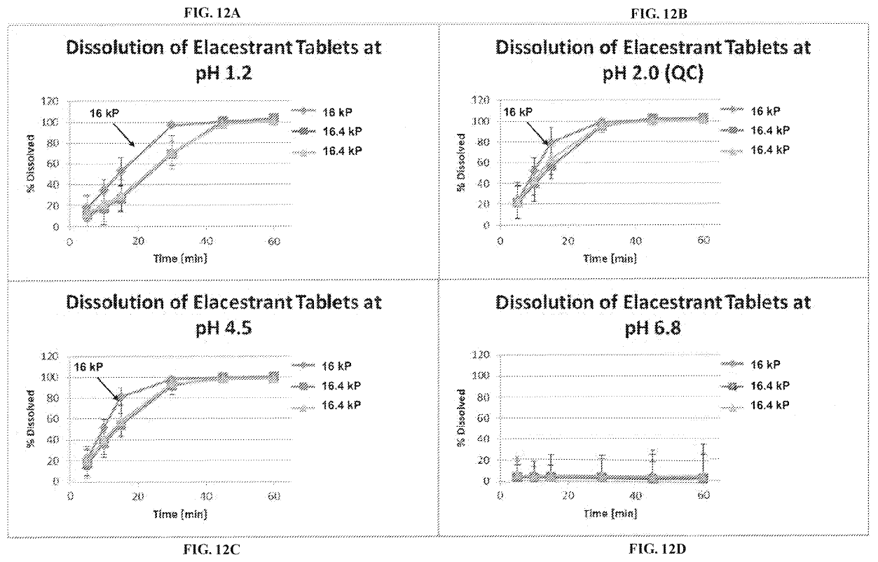

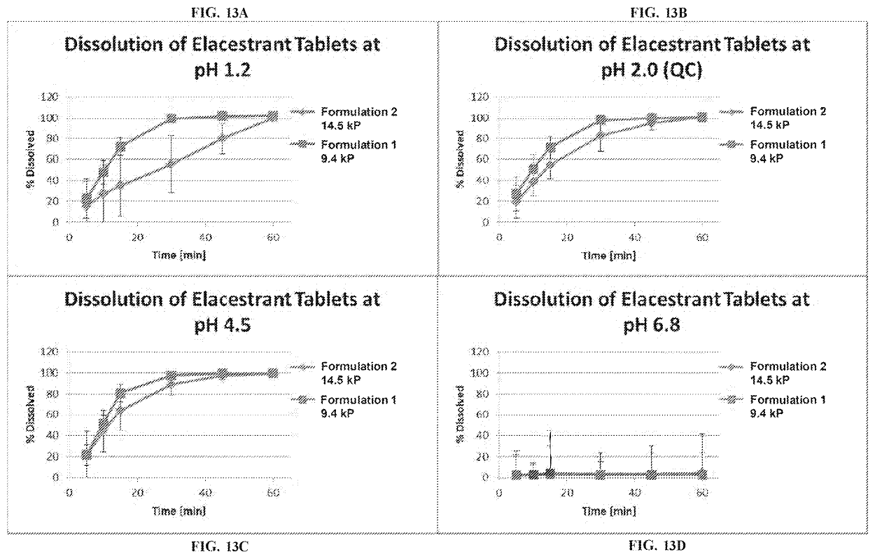

Dissolution profiles for Formulation 2 at the target hardness (13.3 kP and 16.4 kP for 100 mg and 400 mg tablets, respectively), were generated using testing conditions that represented the physiological range of pH 1.2 (0.1 N HCl) to pH 6.8 (phosphate buffer), as well as in the QC medium (0.01 N HCl) and at pH 4.5 (acetate buffer). The dissolution profiles under these various conditions are shown in FIGS. 11A-D (100 mg tablets) and FIGS. 12A-D (400 mg tablets).

Comparison of the dissolution profiles for 100 mg tablet in 4 different media are shown in FIGS. 11A-D. One batch was intentionally compressed at a higher compression force, resulting in tablets with higher tablet hardness (14.5 kP). This was done to determine the impact of hardness on dissolution and bioavailability. The dissolution profiles at both pH 1.2 and pH 2.0 for this batch exhibit slower dissolution than the other two batches that were compressed at a lower hardness, demonstrating the discriminatory capability of the dissolution method under these conditions. At pH 4.5, the dissolution profiles did not separate, as was observed at the lower pH values, indicating that at this pH, the dissolution conditions were not capable of discriminating the differences among these batches. At the highest tested pH (pH 6.8), the solubility of elacestrant dihydrochloride is significantly lower, and therefore this test condition is not a reliable method for testing the dissolution of elacestrant dihydrochloride 100 mg tablets. The data in FIG. 11 show that the QC dissolution method can detect differences between batches (in this case tablets at the high end of the hardness range).

FIGS. 12A-D show similar data, only in this case for the elacestrant dihydrochloride 400 mg tablets. Here, one batch was compressed at a lower hardness (16 kP) to determine the impact of hardness on dissolution and subsequent bioavailability. The dissolution profiles at both pH 1.2 and pH 2.0 for this batch exhibit faster dissolution than the other two batches that were compressed at a higher hardness (16.4 kP), demonstrating the discriminatory capability of the dissolution method under these conditions. At pH 4.5, the dissolution profiles of the 400 mg tablets also showed the ability to separate the dissolution curves, similar to what was observed at the lower pH values. Again, at the highest tested pH (pH 6.8), the solubility of elacestrant dihydrochloride is so low that this test condition is not a reliable method for testing the dissolution of elacestrant dihydrochloride 400 mg tablets. The data in FIGS. 12A-D show that the QC dissolution method is capable of detecting differences among batches. It should be noted that, in a Pilot PK Study, elacestrant dihydrochloride 400 mg tablets compressed at the high end of the hardness range were found to be bioequivalent to the Phase 3 clinical trial tablet, again indicating that the dissolution method is overly discriminating when compared to actual bioequivalence data.

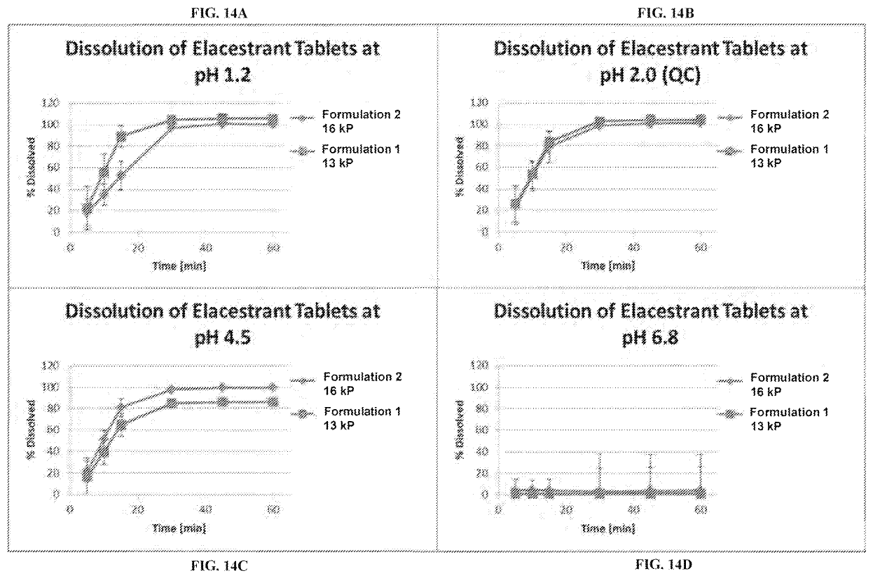

One batch per dosage strength was selected for a study to confirm the bioequivalence of Formulation 2 to the Phase 3 formulation (Formulation 1). For the 100 mg strength, the Formulation 2 batch was intentionally compressed to a higher hardness (14.5 kP) to produce tablets at the low end of the dissolution range and establish a better understanding of the relationship between tablet hardness and bioavailability. The 400 mg strength the batch was compressed to a hardness (16 kP) which would generate a dissolution profile that fell between the dissolution profiles for the Pilot PK batches, that had already demonstrated bioequivalence in the Pilot PK Study. Comparison of the dissolution profiles at various media conditions are shown in FIGS. 13A-D (100 mg tablets) and FIGS. 14A-D (400 mg tablets).

FIGS. 13A-D compare the dissolution profiles of the elacestrant dihydrochloride 100 mg drug product used in the Pivotal BE study under 4 different dissolution conditions. The dissolution profiles at both pH 1.2 and pH 2.0 for this batch exhibit slower dissolution than the Phase 3 Comparator batch (Formulation 1). At pH 4.5, the dissolution profiles showed some separation, not as significant as the separation observed at the lower pH values, indicating that the dissolution test conditions were not capable of discriminating the difference between these batches. At the highest tested pH (pH 6.8) the solubility of the drug is so low that this test condition is not a reliable method for testing the dissolution of elacestrant dihydrochloride 100 mg tablets. The curves were compared using f2 analysis. The f2 values at pH 1.2 and 2.0 showed that the two dissolution curves were not similar (f2<50), but at pH 4.5 they were similar (f2>50). Although the data at pH 6.8 also indicate the two curves are similar, the percent released was so low that this comparison was not of any value. The data in FIGS. 13A-D show that the QC dissolution method is capable of detecting differences between batches (in this case tablets at the high end of the hardness range), but these differences were not reflected in the outcome of the Pivotal BE study.

FIGS. 14A-D compare the dissolution profiles of the elacestrant 400 mg tablets used in the Pivotal BE study under 4 different dissolution conditions. The elacestrant 400 mg Formula 3 batch was compared to the Pivotal Phase 3 batch (Formulation 1). The dissolution profiles at pH 1.2 showed some separation between curves, but at pH 2.0 there was no separation of curves. The pH 4.5 dissolution profiles also showed some separation of the dissolution curves. Again, at the highest tested pH (pH 6.8), the solubility of the drug is so low that this test condition is not a reliable method for testing the dissolution of elacestrant dihydrochloride 400 mg tablets. These curves were compared using f2 analysis. The f2 values showed that the two dissolution curves were not similar (f2<50), but at pH 2.0 they were similar (f2≥50). The data in FIGS. 14A-D show that differences could be observed between the dissolution profiles based on the method used. However, these differences were not reflected in the outcome of the Pivotal BE study.

### Example 6. PBBM Model Development for Virtual Bioequivalence Trials

A physiologically based biopharmaceutics model (PBBM) was developed using the GastroPlus® software (v9.8.2) and ADMET Predictor® (v.10.3) for virtual bioequivalence trials to show safety and efficacy of the tablet formulations according to the present disclosure. The PBBM model was developed and based on 31 clinical scenarios (including 15 fit-for-purpose clinical scenarios out of 4 clinical trials). The PBBM incorporated drug product dissolution in a mechanistic way, using the product hydrodynamic particle size distribution (P-PSD HD) approach, which was able to predict the dissolution profile of the same batch in different media.

A proposed dissolution acceptance criterion of Q=80% at 45 min in the FDA approved QC methods for 100 mg and 400 mg tablets is deemed justified based on: (1) the results of virtual bioequivalence (VBE) studies using GastroPlus®; and (2) the exposure-response (ER) data available for elacestrant. Specifically, the proposed dissolution acceptance criterion for elacestrant tablets, 100 mg and 400 mg, are justified based on the establishment of strength dependent dissolution safe spaces. The upper limit of the safe space is based on dissolution of batches tested in pivotal clinical trials and thus, considered self-evident. The lower limits are based on model predictions of virtual batches' dissolution profiles (virtual batches A and B) and further supported by ER information.

Virtual Batch A's (VBA's) dissolution profile is representative of a batch meeting the Q=80% at 45 min acceptance criterion. Virtual Batch B's (VBB's) profile is more permissive and is representative of a batch meeting the Q=75% at 45 min acceptance criterion. These virtual batches were shown to be bioequivalent to the reference batches (representative of pivotal Phase 3 trials) in the fasted and fed state. Repeat dose simulation for 400 mg VBB (representative of Q=75% in 45 min) in the fed state shows that 90% of the population will display effective plasma elacestrant concentrations since the Cₘᵢₙ at steady state is higher than the threshold of 20 ng/mL. In addition, repeat dose simulations for 100 mg VBB (representative of Q=75% in 45 min) in the fed state show that the Cₘᵢₙ at steady state is comparable to that observed for 100 mg Formulation 1 (Phase 3 clinical reference) and other pivotal clinical data. The lower bounds of the dissolution safe space are further supported by ER analysis, which showed that neither elacestrant exposure nor any of the covariates tested reached the predefined level of significance during non-parametric and parametric survival analysis for PFS (progression-free survival), indicating no differences in PFS across the observed range of elacestrant exposures.

### Modeling Strategy

The PBBM was built based on measured and predicted physicochemical and biopharmaceutical properties of elacestrant. The metabolic clearance was specified based on clinical PK data following one minute infusion of 1 mg elacestrant in study RAD1901-001 and in silico predictions of Michaelis-Menten constant (Km) values (ADMET Predictor® v.10.3) for CYP3A4, a major enzyme involved in the drug metabolism. A physiologically based pharmacokinetic (PBPK) model was applied based on the Lukacova model to calculate the tissue to plasma partition coefficients. The dissolution was mechanistically integrated based on the QC dissolution methods for 100 mg and 400 mg tablets. The P-PSD HD approach is retained for 100 mg and 400 mg tablets. The P-PSD HD approach was validated (fit-for-purpose) on two batches for which dissolution was performed in multiple media and then applied to all clinical and virtual tablet batches to extract a batch specific input to the model. In addition, a mechanistic understanding of drug precipitation was integrated in the model and fitted to the oral PK profile obtained in the fasted state following single dose administration of elacestrant 200 mg in healthy volunteers.

The PBBM was validated based on 31 clinical scenarios from several studies in healthy volunteers and patients with doses ranging from 10 to 1000 mg (capsule formulation) and 100 mg and 400 mg (tablet formulation and their variants). The model was also validated in various prandial states using fasted, low, and high-fat diets and with and without proton pump inhibitor treatment. Sensitivity analyses were run on the validated model to identify main sources of intra- and intersubject variability and explain the limitations to drug absorption. Using the validated PBBM, the virtual dissolution profiles of tablets, 100 mg and 400 mg and VBE studies, the edge of safe space for drug product dissolution was determined. Based on the safe space, a clinically relevant dissolution acceptance criterion of Q=80% at 45 min is evident for both 100 mg and 400 mg tablets.

### Summary of Physicochemical and Biopharmaceutical Properties for Elacestrant Dihydrochloride and Elacestrant (Free Base)

The physicochemical and biopharmaceutical properties for elacestrant dihydrochloride and its free base, elacestrant, were defined using a combination of in silico estimates from the ADMET Predictor® module that were based on the chemical structure, along with in vitro and in vivo data obtained from the literature and internal data. Table 5 summarizes the parameter values used for the elacestrant dihydrochloride pharmaceutically based pharmacokinetics (PBPK) model.

The solubility vs. pH profile is solved using conventional methods known to those in the art and using the measure pKa values for elacestrant (Table 5). The pHₘₐₓ for elacestrant is 4.3. These values were used to model in vitro drug product dissolution. The in vivo solubility values of elacestrant dihydrochloride are calculated as a function of the volume administered and the food volume, where relevant.

Various formulations were used for development. For the purpose of the PBBM, only drug substance in capsules and tablets from Formulation 1 and Formulation 2 were used. (See Table 3.)

TABLE 5
Input Parameters for Elacestrant Dihydrochloride PBPK Model Development

| | | |
|---|---|---|
| | Value for Elacestraut | |
| PBPK Parameter | Dihydrochloride | Rationale/Reference(s) |

1. Physicochemical and Binding Properties

| | | |
|---|---|---|
| Molecular mass (g/mol) | [symbol] | From structure |
| Salt to free base ratio | 1.159 | From structure |
| Type of drug substance | Salt is crystalline | |
| Log P | 5.56 | Measured |
| pKa | 6.39(B), 7.74(B), 11.98(A) | Measured |
| Precipitation time ( [symbol] ) | Mechanistic model | |
| | Lindfors | |
| | Lambda = 50 μm | |
| | Sigma = 0.006 J · m⁻² | |
| Intrinsic solubility (mg/mL) | Free base: 1.15E−4 | Calculated from measured values on |
| | (pH = 10) | the free base A |
| | Salt: 39.4 (pH = 4.3) | |
| Human blood-to-plasma | 2.5 | Initial value of 0.701 in RAD1901-111, |
| ratio (R [symbol] ) | | adjusted to observed distribution in |
| | | IV PK profile generated in RAD1901-001 |
| [symbol] plasma | 0.01 (Human) | 21RAD233 |
| | 0.0161 (Rat) | |
| | 0.00919 (Monkey) | |

2. Absorption

| | | |
|---|---|---|
| Human effective jejunal | 0.6 | Initial value of 0.32 10 [symbol] cm/s |
| permeability (P [symbol] ) | | scaled from in vitro Caco2 data of |
| (×10⁻⁴ cm/s) | | 0.75 10 [symbol] cm/s compared to atenolol |
| | | values of 0.47 10 [symbol] cm/s (16RAD200) |
| | | and using human measured P [symbol] for |
| | | atenolot of 0.2 10 [symbol] cm/s [1]. |
| | | Value adjusted to 0.6 10 [symbol] cm/s |
| | | based on human PK profiles observed in |
| | | RAD1901-001 |
| Dissolution model | Johnson | Default, using P-PSD for each batch fitted |

2. Absorption

| | | |
|---|---|---|
| Human effective jejunal | 0.6 | Initial value of 0.32 10 [symbol] cm/s |
| permeability (P [symbol] ) | | scaled from in vitro Caco2 data of |
| (×10× cm/s) | | 0.75 10 [symbol] cm/s compared to atenolol |
| | | values of 0.47 10 [symbol] cm/s (16RAD200) |
| | | and using human measured P [symbol] for |
| | | atenolol of 0.2 10 [symbol] cm/s [1]. |
| | | Value adjusted to 0.6 10 [symbol] cm/s |
| | | based on human PK profiles observed in |
| | | RAD1901-001 |
| Dissolution model | Johnson | Default, using P-PSD for each batch fitted |

3. Distribution

| | | |
|---|---|---|
| Method | Full body PBPK, Lukacova | GastroPlus default approach |
| | method of K [symbol] prediction | |
| | for all tissues | |

4. Metabolism

| | | |
|---|---|---|
| CYP3A4 K [symbol] (mg/l) | 2.586 | APv10.3 |
| PSPK CYP3A4 V [symbol] (mg/s/mg-enz) | 0.0304 | Fitted to 1 mg IV 1 min infusion in RAD1901-001 |
| Gut CYP3A4 V [symbol] (mg/s) | 1 | Fitted to oral 10 mg data in RAD1901-001 |

[symbol]

indicates data missing or illegible when filed

### Integration of Dissolution in the PBBM

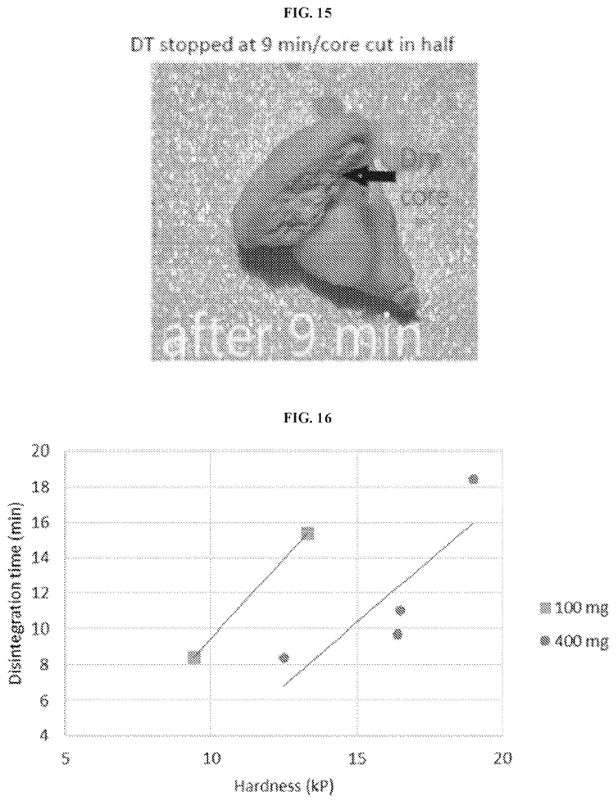

To select the model that best predicts the in vivo dissolution for the different formulations or virtual batches of elacestrant dihydrochloride tablets, the failure modes impacting dissolution of the elacestrant tablets were analyzed. Despite the presence of disintegrants in the formulations of elacestrant dihydrochloride tablets, it was observed that above a certain compression force threshold, the tablet surprisingly does not disintegrate immediately in contact with water. Instead, a gelling layer has been observed on the surface of elacestrant dihydrochloride tablets which prevents rapid permeation by water into the tablet—and subsequent disintegration—at most tablet compression forces and appears to drive the mechanism of drug release. FIG. 15 shows an elacestrant dihydrochloride tablet for which dissolution was stopped and the core was cut in half after 9 minutes. The tablet exhibits a dry core surrounded by a smooth, gel-like layer. Thus, erosion is the presumed mechanism of dissolution in the elacestrant dihydrochloride tablets according to the present disclosure.

The tablet compression force and tablet hardness increase the disintegration time measured on 100 mg or 400 mg tablets. Since the shape of the tablet is different between the two dosage strengths, the relationship of disintegration time and tablet hardness depends on the dose of the tablet (shape of the tablet), as shown in Table 6 and FIG. 16.

TABLE 6
Hardness and Disintegration Times Measured on 100
mg and 400 mg Elacestrant Dihydrochloride Tablets

| | Dose | Hardness | Disintegration |
|---|---|---|---|
| Formulation | (mg) | (kP) | Time (min.) |
| 2 | 100 | 9.4 | 8.4 |
| 2 | 100 | 13.3 | 15.4 |
| 2 | 400 | 16.5 | 11.0 |
| 2 | 400 | 16.4 | 9.7 |
| 2 | 400 | 12.5 | 8.4 |
| 2 | 400 | 19 | 18.4 |

Referring to FIG. 16, the tablet disintegration time is clearly slower for a 100 mg than for a 400 mg tablet for an equivalent tablet hardness. This is related to the lower surface area available for the 100 mg tablet, which will lead to less erosion than for the 400 mg tablet. A relationship can be drawn between tablet dissolution and main critical quality attributes of the tablets. Since the release mechanism in vitro of elacestrant tablets appears to be through erosion, there seems to be a limited impact of DS particle size on dissolution compared to the impact of tablet hardness. Thus, for 100 mg and 400 mg batches tested in vitro using the QC dissolution methods (USP2, 75 rpm, 37° C., 0.01 N HCl pH 2 with 500 mL for 100 mg tablets and 1000 mL for 400 mg tablets), the percent dissolved at 5 min or 15 min can be correlated to the hardness of the tablets.

To this end, Table 7 summarizes the values for tablet hardness and percent dissolved at 5 min and 15 min in the QC methods (most discriminant time points) for development and clinical batches obtained at different hardness values. For an eroding tablet (presumed mechanism of elacestrant tablets drug release), the surface-to-volume (S/V) ratio of the tablet strength would be expected to increase the percent dissolved at any time point since more surface area per drug load would be available for erosion and dissolution. Additionally, an increase in the tablet hardness would be expected to reduce the tablet dissolution rate since, by closing the tablet porosity, the progression of the waterfront into the tablet would be slower. Hence, these two quality attributes can be included in the model. The S/V ratio of the 100 mg and 400 mg tablets across batches of Formulations 1-2 was estimated at 0.943 mm⁻¹ and 0.562 mm⁻¹, respectively. The 100 mg tablet S/V ratio divided by the 400 mg tablet S/V ratio is therefore estimated at 1.68.

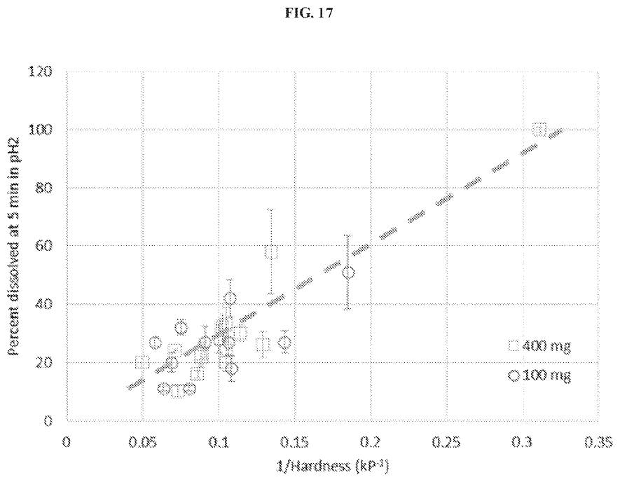

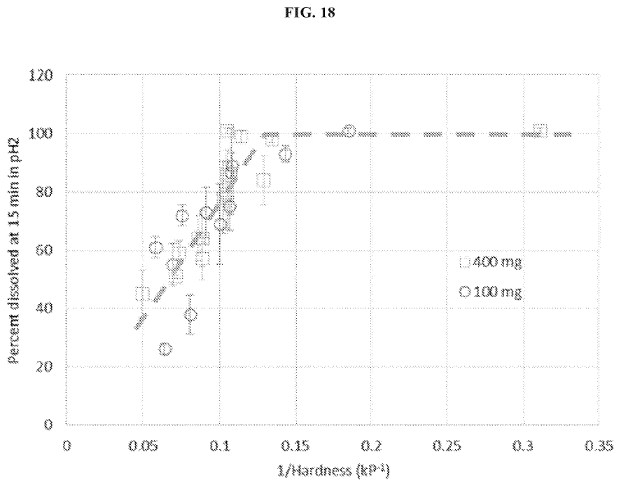

FIG. 17 illustrates the relationship between percent dissolved at 5 min and the inverse of the tablet hardness for all tablets tested, as shown in Table 7. Meanwhile, FIG. 18 illustrates the relationship between percent dissolved at 15 min and the inverse of the tablet hardness for all tablets tested, as shown in Table 7. To establish the correlation, the parameters shown in FIG. 17 and FIG. 18, the inverse hardness values for the 400 mg tablets is multiplied by a factor of 1.68 to enable comparison to the 100 mg tablet data.

TABLE 7
Measured Hardness and Percent Dissolved at 5 min and 15
min for Formulations 1-2, 100 mg and 400 mg Tablets

| | | | Percent | Standard | Percent | Standard |
|---|---|---|---|---|---|---|
| | Dose | Hardness | Dissolved, 5 min | Deviation | Dissolved, 15 min | Deviation |
| Formulation | (mg) | (kP) | (%) | (%) | (%) | (%) |
| 2 | 100 | 6.98 | 27 | 3.8 | 93 | 2.8 |
| 2 | 100 | 9.24 | 18 | 4.3 | 89 | 4.5 |
| 2 | 100 | 12.44 | 11 | 0.9 | 38 | 6.8 |
| 2 | 100 | 15.74 | 11 | 0.8 | 26 | 1.8 |
| 2 | 100 | 5.41 | 51 | 12.8 | 101 | 1.0 |
| 2 | 100 | 9.32 | 42 | 6.3 | 87 | 14.8 |
| 2 | 100 | 13.3 | 32 | 2.6 | 72 | 3.6 |
| 2 | 100 | 17.28 | 27 | 1.4 | 61 | 3.7 |
| 1 | 100 | 9.4 | 27 | 4.6 | 75 | 8.3 |
| 2 | 100 | 14.5 | 20 | 3.2 | 55 | 7.2 |
| 2 | 100 | 11 | 27 | 5.4 | 73 | 8.8 |
| 2 | 100 | 10 | 28 | 4.8 | 69 | 13.8 |
| 2 | 400 | 5.4 | 100 | 1.0 | 101 | 1.0 |
| 2 | 400 | 16.4 | 32 | 4.5 | 78 | 12.5 |
| 2 | 400 | 23.54 | 24 | 0.5 | 51 | 0.5 |
| 2 | 400 | 33.9 | 20 | 2.2 | 45 | 8.1 |
| 1 | 400 | 13 | 26 | 4.4 | 84 | 8.4 |
| 1 | 400 | 14.7 | 30 | 3.0 | 99 | 2.0 |
| 2 | 400 | 16 | 33 | 5.9 | 101 | 1.0 |
| 2 | 400 | 23 | 10 | 2.0 | 59 | 4.1 |
| 2 | 400 | 16 | 24 | 4.1 | 79 | 11.9 |
| 2 | 400 | 19 | 22 | 3.5 | 57 | 7.4 |
| 2 | 400 | 19 | 23 | 3.7 | 64 | 9.6 |
| 2 | 400 | 12.5 | 58 | 14.5 | 98 | 1.0 |
| 2 | 400 | 16 | 20 | 2.8 | 86 | 8.6 |
| 2 | 400 | 19.5 | 16 | 1.4 | 64 | 7.7 |

FIG. 17 shows that for very low tablet hardness values, the tablet will disintegrate rapidly and provide full release at the first time point of measurement (5 min). This was observed for the 400 mg tablet batch of Formulation 2 with a tablet hardness of 5.4 kP. It is estimated from the extrapolation to 100% dissolution at 5 min that the minimum 100 mg tablet hardness below which full disintegration will occur is 3.1 kP. For these tablets with relatively low hardness, the release mechanism compared to similar batches with higher hardness therefore changed to disintegrating tablets, rather than eroding tablets.

FIG. 18 shows that the mechanism of release of elacestrant dihydrochloride tablets and variants is through tablet erosion, which is in turn a function of the tablet S/V ratio and tablet hardness. The P-PSD approach will capture the effect of tablet hardness on the slower erosion of certain formulations. The 100 mg tablets compared to 400 mg tablets will display a lower P-PSD for an equivalent tablet hardness due to S/V considerations.

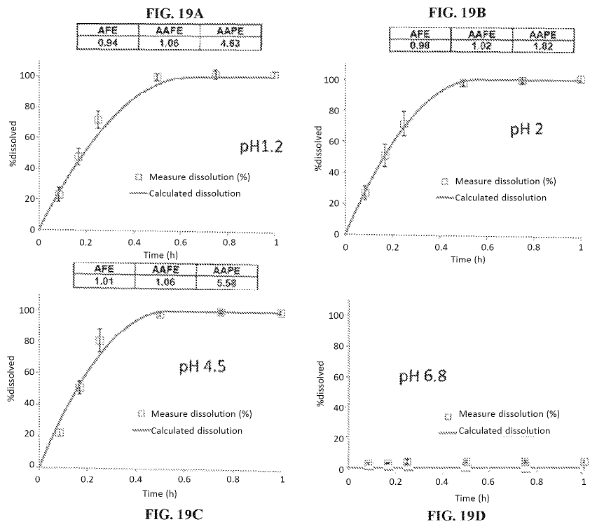

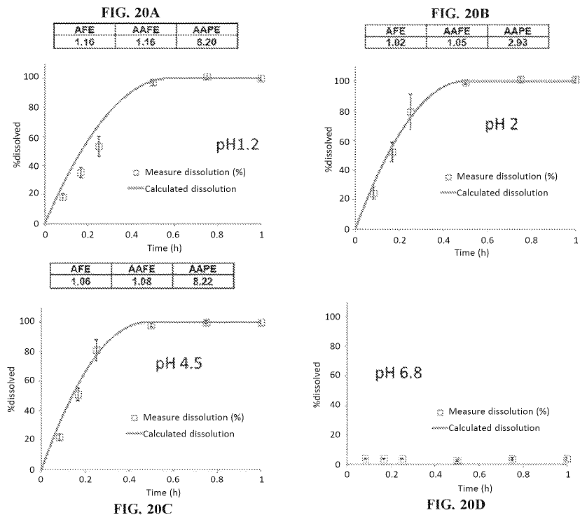

The P-PSD HD approach described by Pepin et al. was utilized for tablets due to the erosion mechanism for drug release. See X. J. H. Pepin, et al., Mechanistic Models for USP2 Dissolution Apparatus, Including Fluid Hydrodynamics and Sedimentation, 111 J. PHARM. SCI. 185 (2021). Indeed, there is no rapid disintegration for 100 mg or 400 mg elacestrant dihydrochloride tablets, and the tablet shape and hardness are the major drivers for drug dissolution. Since the mechanism of release appears to be driven by erosion, the hydrodynamic P-PSD approach was implemented, taking into consideration the effect of agitation in the USP2 apparatus. For larger particles or eroding tablets the effect of agitation needs to be considered. See id.; see also X. J. H. Pepin, et al., Physiologically Based Biopharmaceutics Model for Selumetinib Food Effect Investigation and Capsule Dissolution Safe Space—Part I: Adults, 40 PHARM. RES. 387 (2023). The dissolution rate of selected batches of elacestrant dihydrochloride 100 mg and 400 mg batches at different pH levels is shown in FIGS. 19A-D and FIGS. 20A-D to demonstrate the suitability of this modeling approach.

### Example 7. Model Application for Virtual Bioequivalence Studies

Two separate sets of virtual BE (VBE) studies are conducted to define safe spaces for 100 mg and 400 mg tablets, using the PBBM model discussed in Example 6. Because the QC dissolution methods are distinct for 100 mg and 400 mg tablets, two safe spaces need to be defined. Four elacestrant dihydrochloride virtual batches were thus defined: 100 mg VBA; 100 mg VBB; 400 mg VBA; and 400 mg VBB.

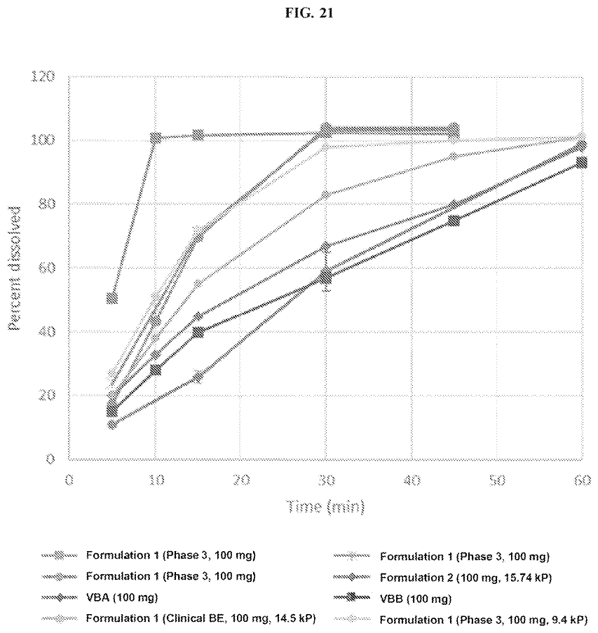

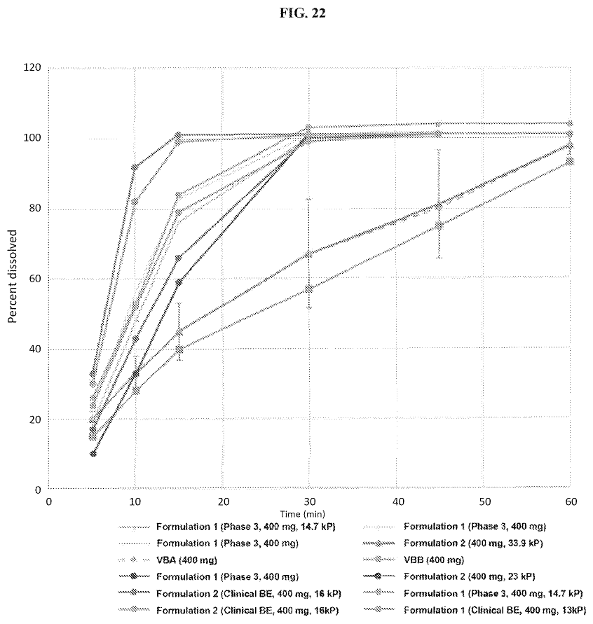

Regarding the dissolution profiles of the virtual batches, VBA meets the criterion of Q=80% at 45 min, and VBB the criterion of Q=80% at 50 min in both dissolution methods for the respective strengths. The dissolution profile of 100 mg batches is illustrated in FIG. 21. The dissolution profile of 400 mg batches is illustrated in FIG. 22.

Referring to FIG. 21, although outside the knowledge space for building the safe space, the dissolution of 100 mg VBA and VBB batches is not an extrapolation of the 100 mg tablet proven ranges of the design space, since the 100 mg batch of Formulation 2, tableted to 15.74 kP, dissolves slower than both 100 mg VBA and VBB until 80% dissolution is achieved in the 100 mg tablet QC method. Similarly, referring now to FIG. 22, for the 400 mg strength, although outside the knowledge space for building the safe space, the dissolution of 400 mg VBA and VBB batches is not an extrapolation of the 400 mg tablet proven ranges of the design, since the 400 mg batch of Formulation 2, tableted to 33.9 kP, has an equivalent dissolution rate to 400 mg VBA. The variability observed in the dissolution Formulation 2 (33.9 kP) also encompasses the VBB profile until 90% dissolution is achieved in the 400 mg tablet QC method.

### Design of Virtual Bioequivalence (VBE) Studies

All VBE studies conducted to define safe spaces for 100 mg and 400 mg elacestrant tablets against chosen Phase 3 pivotal batch references (Formulation 1) is shown in Table 8.

TABLE 8
VBE Studies Conducted to Establish Lower Edge of Safe Space

| VBE Study | Tablet | | Test | Number | Number of | Prandial |
|---|---|---|---|---|---|---|
| Set # | Dose (mg) | Reference Batch | Batch | of Studies | Subjects | State |
| 1 | 100 | Formulation 1 | Actual | 10 | 38 | Fasted |
| 2 | 100 | (Phase 3, 9.4 kP) | VBA | | | |
| 3 | 100 | | VBB | | | |
| 4 | 400 | Formulation 1 | Actual | | | |
| 5 | 400 | (Phase 3, 14-15 kP) | VBA | | | |
| 6 | 400 | | VBB | | | |
| 7 | 100 | Formulation 1 | Actual | | | Low-fat, |
| 8 | 100 | (Phase 3, 9.4 kP) | VBA | | | low- |
| 9 | 100 | | VBB | | | calorie |
| 10 | 400 | Formulation 1 | Actual | | | meal |
| 11 | 400 | (Phase 3, 14-15 kP) | VBA | | | |
| 12 | 400 | | VBB | | | |

The sets of VBE studies #1, #4, #7, and #10 are designed to verify in the fasted and fed state with low-fat, low-calorie meal (LL), whether the Phase 3 clinical reference batches for 100 mg and 400 mg tablets can be demonstrated to be bioequivalent to themselves using n=10 VBE studies with n=38 subjects. The results of the VBE studies are shown in Tables 9-20.

TABLE 9
Results of VBE Set #1

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 101.8 | 97.2 | 106.3 | 101.5 | 99.0 | 104.0 | 101.5 | 99.0 | 104.0 |
| Study 2 | 99.5 | 95.3 | 103.6 | 99.7 | 97.2 | 102.2 | 99.7 | 97.2 | 102.2 |
| Study 3 | 98.7 | 94.2 | 103.2 | 98.8 | 96.4 | 101.2 | 98.8 | 96.3 | 101.2 |
| Study 4 | 97.0 | 93.7 | 100.3 | 97.9 | 95.9 | 99.8 | 97.9 | 95.9 | 99.8 |
| Study 5 | 103.6 | 99.5 | 107.6 | 102.5 | 100.6 | 104.5 | 102.5 | 100.5 | 104.5 |
| Study 6 | 97.1 | 93.2 | 101.0 | 98.9 | 97.0 | 100.8 | 98.9 | 96.9 | 100.8 |
| Study 7 | 102.2 | 98.1 | 106.3 | 101.2 | 99.1 | 103.3 | 101.2 | 99.0 | 103.3 |
| Study 8 | 101.0 | 96.4 | 105.7 | 101.0 | 98.5 | 103.6 | 101.0 | 98.5 | 103.6 |
| Study 9 | 102.3 | 97.4 | 107.2 | 101.6 | 99.1 | 104.1 | 101.6 | 99.1 | 104.2 |
| Study 10 | 103.2 | 99.9 | 106.6 | 102.9 | 101.0 | 104.9 | 102.9 | 100.9 | 104.9 |

TABLE 10
Results of VBE Set #2

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 90.3 | 86.0 | 94.5 | 92.1 | 90.0 | 94.1 | 92.0 | 89.9 | 94.1 |
| Study 2 | 93.1 | 89.5 | 96.7 | 91.7 | 89.7 | 93.8 | 91.7 | 89.7 | 93.8 |
| Study 3 | 87.2 | 83.7 | 90.7 | 89.2 | 87.3 | 91.1 | 89.2 | 87.2 | 91.1 |
| Study 4 | 91.1 | 87.0 | 95.2 | 91.3 | 89.1 | 93.5 | 91.3 | 89.1 | 93.5 |
| Study 5 | 88.8 | 85.3 | 92.4 | 89.7 | 87.5 | 921.0 | 89.7 | 87.5 | 92.0 |
| Study 6 | 91.0 | 87.3 | 94.7 | 91.5 | 89.4 | 93.5 | 91.4 | 89.4 | 93.5 |
| Study 7 | 93.8 | 90.0 | 97.6 | 93.2 | 91.2 | 95.1 | 93.2 | 91.2 | 95.1 |
| Study 8 | 88.1 | 84.6 | 91.5 | 90.1 | 88.0 | 92.3 | 90.1 | 88.0 | 92.3 |
| Study 9 | 94.2 | 90.2 | 98.2 | 93.3 | 91.2 | 995.4 | 93.3 | 91.2 | 95.4 |
| Study 10 | 88.9 | 84.2 | 93.5 | 90.9 | 88.5 | 93.3 | 90.9 | 88.5 | 93.3 |

TABLE 11
Results of VBE Set #3

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 90.8 | 86.3 | 95.4 | 91.6 | 89.1 | 94.0 | 91.5 | 89.1 | 94.0 |
| Study 2 | 93.4 | 89.9 | 97.0 | 92.1 | 89.7 | 94.5 | 92.1 | 89.7 | 94.5 |
| Study 3 | 86.3 | 82.7 | 90.0 | 88.0 | 85.5 | 90.6 | 88.0 | 85.5 | 90.6 |
| Study 4 | 89.8 | 85.4 | 94.2 | 90.3 | 87.8 | 92.7 | 90.3 | 87.8 | 92.7 |
| Study 5 | 86.1 | 82.8 | 89.4 | 87.9 | 86.0 | 89.9 | 87.9 | 85.9 | 89.9 |
| Study 6 | 87.7 | 85.4 | 89.9 | 89.7 | 88.2 | 91.2 | 89.6 | 88.1 | 91.1 |
| Study 7 | 88.6 | 84.2 | 92.9 | 89.2 | 86.5 | 91.9 | 89.2 | 86.5 | 91.9 |
| Study 8 | 92.4 | 88.5 | 96.3 | 91.4 | 89.3 | 93.5 | 91.4 | 89.3 | 93.5 |
| Study 9 | 90.5 | 85.9 | 95.1 | 90.1 | 87.4 | 92.9 | 90.1 | 87.4 | 92.8 |
| Study 10 | 92.6 | 88.7 | 96.4 | 92.2 | 90.0 | 94.3 | 92.1 | 89.9 | 94.3 |

TABLE 12
Results of VBE Set #4

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 104.6 | 99.1 | 110.1 | 104.4 | 99.4 | 109.5 | 104.4 | 99.4 | 109.5 |
| Study 2 | 102.4 | 96.0 | 108.9 | 101.7 | 96.6 | 106.9 | 101.7 | 96.6 | 106.9 |
| Study 3 | 97.6 | 91.7 | 103.5 | 99.4 | 94.3 | 104.5 | 99.4 | 94.3 | 104.4 |
| Study 4 | 102.5 | 97.7 | 107.4 | 103.1 | 98.8 | 107.4 | 103.1 | 98.8 | 107.4 |
| Study 5 | 106.1 | 102.3 | 109.9 | 107.6 | 102.7 | 112.6 | 107.6 | 102.7 | 112.6 |
| Study 6 | 102.8 | 97.7 | 10718 | 102.8 | 98.4 | 107.1 | 102.7 | 98.4 | 107.1 |
| Study 7 | 104.1 | 97.8 | 110.5 | 103.5 | 97.6 | 109.4 | 103.5 | 97.6 | 109.4 |
| Study 8 | 104.7 | 101.2 | 108.2 | 104.6 | 100.8 | 108.4 | 104.6 | 100.8 | 108.4 |
| Study 9 | 102.8 | 98.5 | 107.0 | 102.9 | 99.1 | 106.7 | 102.9 | 99.1 | 106.7 |
| Study 10 | 105.3 | 99.0 | 111.6 | 103.9 | 98.9 | 108.8 | 103.9 | 99.0 | 108.8 |

TABLE 13
Results of VBE Set #5

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 93.5 | 89.1 | 97.8 | 93.8 | 89.9 | 97.8 | 93.8 | 89.9 | 97.8 |
| Study 2 | 90.5 | 86.3 | 94.7 | 90.4 | 87.1 | 93.8 | 90.4 | 87.1 | 93.8 |
| Study 3 | 91.8 | 87.5 | 96.1 | 92.3 | 87.9 | 96.7 | 92.3 | 87.9 | 96.8 |
| Study 4 | 92.3 | 89.4 | 95.2 | 91.5 | 88.8 | 94.1 | 91.4 | 88.8 | 94.1 |
| Study 5 | 94.7 | 88.8 | 100.6 | 95.3 | 89.8 | 100.8 | 95.3 | 89.8 | 100.8 |
| Study 6 | 93.2 | 88.9 | 97.6 | 93.1 | 89.8 | 96.3 | 93.0 | 89.8 | 96.3 |
| Study 7 | 91.8 | 87.2 | 96.4 | 92.4 | 87.8 | 97.0 | 92.4 | 87.8 | 97.0 |
| Study 8 | 93.6 | 89.2 | 97.9 | 93.1 | 89.3 | 96.9 | 93.1 | 89.3 | 96.9 |
| Study 9 | 91.9 | 86.1 | 97.8 | 91.9 | 87.1 | 96.7 | 91.9 | 87.1 | 96.7 |
| Study 10 | 96.8 | 91.6 | 102.0 | 95.7 | 91.0 | 100.3 | 95.7 | 91.0 | 100.3 |

TABLE 14
Results of VBE Set #6

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 93.1 | 88.8 | 97.5 | 93.2 | 89.5 | 96.9 | 93.2 | 89.5 | 96.9 |
| Study 2 | 91.7 | 87.8 | 95.7 | 92.7 | 88.9 | 96.5 | 92.7 | 88.9 | 96.5 |
| Study 3 | 93.7 | 89.1 | 98.3 | 93.0 | 88.7 | 97.3 | 93.0 | 88.7 | 97.3 |
| Study 4 | 93.0 | 88.5 | 97.6 | 93.2 | 88.7 | 97.7 | 93.2 | 88.7 | 97.7 |
| Study 5 | 92.2 | 88.9 | 95.6 | 92.7 | 89.5 | 95.8 | 92.7 | 89.5 | 95.8 |
| Study 6 | 92.4 | 87.5 | 97.3 | 92.8 | 88.6 | 97.0 | 92.8 | 88.6 | 97.0 |
| Study 7 | 92.6 | 87.3 | 97.8 | 91.6 | 87.1 | 96.2 | 91.6 | 87.1 | 96.2 |
| Study 8 | 92.2 | 88.3 | 96.2 | 92.8 | 89.3 | 96.4 | 92.8 | 89.3 | 96.4 |
| Study 9 | 93.2 | 89.0 | 97.4 | 93.7 | 90.4 | 97.1 | 93.7 | 90.4 | 97.1 |
| Study 10 | 94.3 | 91.5 | 97.1 | 94.5 | 91.7 | 97.2 | 94.5 | 91.7 | 97.2 |

TABLE 15
Results of VBE Set #7

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 96.4 | 92.5 | 100.4 | 97.1 | 94.2 | 100.1 | 97.1 | 94.2 | 100.1 |
| Study 2 | 97.2 | 93.4 | 101.0 | 98.0 | 95.4 | 100.6 | 98.0 | 95.4 | 100.6 |
| Study 3 | 101.3 | 97.1 | 105.4 | 101.1 | 98.3 | 103.9 | 101.1 | 98.3 | 103.9 |
| Study 4 | 95.0 | 0.1 | 99.8 | 96.4 | 92.9 | 99.9 | 96.4 | 92.9 | 99.9 |
| Study 5 | 101.9 | 97.1 | 106.7 | 101.2 | 97.8 | 104.6 | 101.2 | 97.8 | 104.6 |
| Study 6 | 103.7 | 100.1 | 107.2 | 1025 | 99.7 | 105.3 | 102.5 | 99.7 | 105.4 |
| Study 7 | 100.1 | 96.4 | 103.8 | 100.0 | 97.4 | 102.5 | 100.0 | 97.4 | 102.5 |
| Study 8 | 98.6 | 94.7 | 102.5 | 99.2 | 96.3 | 102.0 | 99.2 | 96.4 | 102.0 |
| Study 9 | 105.9 | 101.7 | 110.1 | 103.3 | 100.2 | 106.5 | 103.3 | 100.2 | 106.5 |
| Study 10 | 96.2 | 92.3 | 100.2 | 96.4 | 93.5 | 99.3 | 96.4 | 93.5 | 99.3 |

TABLE 16
Results of VBE Set #8

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 96.4 | 93.8 | 98.9 | 97.5 | 95.5 | 99.4 | 97.4 | 95.5 | 99.4 |
| Study 2 | 96.8 | 93.8 | 99.8 | 97.0 | 94.8 | 99.3 | 97.0 | 94.8 | 99.3 |
| Study 3 | 100.9 | 98.3 | 103.5 | 100.0 | 98.1 | 101.8 | 100.0 | 98.1 | 101.8 |
| Study 4 | 98.6 | 95.6 | 101.6 | 98.7 | 96.5 | 100.9 | 98.7 | 96.5 | 100.9 |
| Study 5 | 97.9 | 94.9 | 101.0 | 98.1 | 95.6 | 100.7 | 98.1 | 95.6 | 100.7 |
| Study 6 | 99.3 | 95.0 | 103.6 | 98.7 | 9.2 | 102.2 | 98.7 | 95.2 | 102.2 |
| Study 7 | 96.7 | 93.2 | 100.2 | 95.8 | 992.8 | 98.8 | 95.8 | 92.8 | 98.8 |
| Study 8 | 96.3 | 92.7 | 99.9 | 97.7 | 95.1 | 100.4 | 97.7 | 95.1 | 100.4 |
| Study 9 | 98.7 | 95.7 | 101.7 | 98.9 | 96.7 | 101.2 | 98.9 | 96.7 | 101.2 |
| Study 10 | 96.2 | 93.6 | 98.9 | 97.8 | 95.6 | 100.0 | 97.8 | 95.6 | 100.0 |

TABLE 17
Results of VBE Set #9

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 97.8 | 94.8 | 100.9 | 97.9 | 95.5 | 100.4 | 97.9 | 95.5 | 100.4 |
| Study 2 | 95.7 | 92.6 | 98.7 | 97.0 | 94.5 | 99.6 | 97.0 | 994.5 | 99.6 |
| Study 3 | 101.2 | 94.5 | 108.0 | 99.9 | 95.1 | 104.7 | 99.9 | 95.1 | 104.7 |
| Study 4 | 96.9 | 93.9 | 99.9 | 98.2 | 95.7 | 100.6 | 98.2 | 95.7 | 100.6 |
| Study 5 | 99.3 | 96.7 | 101.9 | 98.8 | 96.7 | 100.8 | 98.8 | 96.7 | 100.8 |
| Study 6 | 99.2 | 95.5 | 102.9 | 98.8 | 96.1 | 101.4 | 98.7 | 96.1 | 101.4 |
| Study 7 | 99.2 | 95.7 | 1021.17 | 97.7 | 94.8 | 100.6 | 97.7 | 94.8 | 100.6 |
| Study 8 | 93.5 | 89.7 | 97.4 | 94.8 | 91.6 | 97.9 | 94.8 | 91.6 | 97.9 |
| Study 9 | 95.8 | 92.3 | 99.4 | 96.6 | 93.4 | 99.8 | 96.6 | 93.4 | 99.8 |
| Study 10 | 99.5 | 96.0 | 103.0 | 97.9 | 94.9 | 100.9 | 97.9 | 94.9 | 100.9 |

TABLE 18
Results of VBE Set #10

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 97.4 | 93.6 | 101.1 | 98.8 | 96.1 | 101.5 | 98.8 | 96.1 | 101.5 |
| Study 2 | 97.5 | 93.9 | 101.0 | 98.2 | 95.7 | 100.8 | 98.2 | 95.7 | 100.8 |
| Study 3 | 99.0 | 95.9 | 102.0 | 100.4 | 98.2 | 102.6 | 100.4 | 98.1 | 102.6 |
| Study 4 | 101.8 | 99.4 | 104.1 | 100.8 | 98.9 | 102.7 | 100.8 | 98.8 | 102.7 |
| Study 5 | 98.0 | 93.9 | 102.1 | 98.8 | 95.9 | 101.8 | 98.8 | 95.8 | 101.8 |
| Study 6 | 97.2 | 93.2 | 101.2 | 97.5 | 94.6 | 100. | 97.5 | 94.6 | 100.5 |
| Study 7 | 99.2 | 96.2 | 102.2 | 99.5 | 97.2 | 101.8 | 99.5 | 97.2 | 101.8 |
| Study 8 | 98.1 | 95.3 | 100.9 | 99.3 | 96.9 | 101.6 | 99.3 | 96.9 | 101.6 |
| Study 9 | 98.5 | 95.9 | 101.2 | 99.0 | 97.0 | 101.0 | 99.0 | 97.0 | 101.0 |
| Study 10 | 99.6 | 97.2 | 102.0 | 100.0 | 98.1 | 101.9 | 100.0 | 98.1 | 101.9 |

TABLE 19
Results of VBE Set #11

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 93.7 | 88.1 | 99.2 | 96.1 | 92.3 | 99.9 | 96.1 | 92.3 | 99.9 |
| Study 2 | 96.6 | 94.0 | 99.1 | 96.9 | 94.7 | 99.1 | 96.9 | 94.7 | 99.1 |
| Study 3 | 97.4 | 95.2 | 99.7 | 98.4 | 96.6 | 100.2 | 98.4 | 96.6 | 100.2 |
| Study 4 | 95.1 | 921.4 | 97.8 | 96.7 | 94.6 | 98.7 | 96.7 | 94.6 | 98.7 |
| Study 5 | 94.8 | 90.9 | 98.7 | 96.0 | 93.2 | 98.7 | 96.0 | 93.2 | 98.7 |
| Study 6 | 99.3 | 96.2 | 102.5 | 99.5 | 97.1 | 101.9 | 99.4 | 97.0 | 101.9 |
| Study 7 | 96.4 | 93.5 | 99.4 | 96.9 | 94.6 | 99.2 | 96.9 | 94.6 | 99.2 |
| Study 8 | 91.0 | 86.2 | 97.8 | 95.2 | 92.1 | 98.3 | 95.2 | 92.1 | 98.3 |
| Study 9 | 93.3 | 89.5 | 97.1 | 95.7 | 93.2 | 98.1 | 95.6 | 93.2 | 98.1 |
| Study 10 | 96.8 | 93.5 | 100.1 | 98.2 | 95.9 | 100.4 | 98.1 | 95.9 | 100.4 |

TABLE 20
Results of VBE Set #12

| | Cₘₐₓ | | | AUC_{inf} | | | AUCₜ | | |
|---|---|---|---|---|---|---|---|---|---|
| | GMR % | L90% CI | H90% CI | GMR % | L90% CI | H90% CI | GMR % | L90% CI | H90% CI |
| Study 1 | 89.3 | 86.4 | 92.2 | 88.1 | 85.8 | 90.5 | 88.1 | 85.7 | 90.5 |
| Study 2 | 89.9 | 87.6 | 92.2 | 89.4 | 87.5 | 91.3 | 89.4 | 87.5 | 91.3 |
| Study 3 | 90.5 | 88.2 | 92.9 | 90.0 | 88.1 | 919 | 90.0 | 88.1 | 91.8 |
| Study 4 | 89.3 | 86.5 | 92.1 | 89.3 | 87.1 | 91.6 | 89.3 | 87.1 | 91.6 |
| Study 5 | 89.7 | 87.9 | 91.4 | 88.4 | 86.8 | 90.0 | 88.4 | 86.8 | 90.0 |
| Study 6 | 89.4 | 86.7 | 92.0 | 88.7 | 86.4 | 90.9 | 88.7 | 86.4 | 90.9 |
| Study 7 | 92.0 | 89.0 | 95.1 | 90.9 | 88.6 | 93.2 | 90.9 | 88.6 | 93.2 |
| Study 8 | 86.2 | 83.9 | 88.5 | 86.8 | 84.4 | 88.8 | 86.8 | 84.7 | 88.8 |
| Study 9 | 88.4 | 85.3 | 91.5 | 88.6 | 86.1 | 91.0 | 88.6 | 86.1 | 91.0 |
| Study 10 | 91.4 | 88.4 | 94.3 | 89.9 | 87.5 | 92.4 | 89.9 | 87.5 | 92. |

Results from VBE studies shown in Tables 9-11 show that 100 mg VBA and 100 mg VBB are anticipated to be bioequivalent to the Phase 3 pivotal clinical reference (100 mg, Formulation 1, 9.4 kP) in the fasted state. Meanwhile the studies shown in Tables 15-17 show that 100 mg VBA and 100 mg VBB are anticipated to be bioequivalent to the Phase 3 pivotal clinical reference (100 mg Formulation 1) in the fed state.

Overall, the average GMR for 100 mg elacestrant dihydrochloride batches for Formulation 1 (100 mg, 9.4 kP), VBA, and VBB show a reduction in the fasted and fed state as expected from their dissolution characteristics, but keeping within BE limits for all the studies conducted. Therefore, 100 mg elacestrant dihydrochloride batches for Formulation 1 (100 mg, 9.4 kP), VBA, and VBB are anticipated to be bioequivalent to each other. (See Table 21.)

TABLE 21
Results of Estimated GMR Test/Reference for
100 mg Tablets in Fasted and Fed State

Average from 10 VBE

| | | Cₘₐₓ | AUC_{inf} | AUCₜ |
|---|---|---|---|---|
| VBE # | Clinical Simulation | Ratio | Ratio | Ratio |
| 1 | 100 mg Formulation 1 | 100.6 | 100.6 | 100.6 |
| | (9.4 kP) fasted | | | |
| 2 | 100 mg VBA fasted | 90.7 | 91.3 | 91.3 |
| 3 | 100 mg VBB fasted | 89.8 | 90.3 | 90.2 |
| 7 | 100 mg Formulation 1 | 99.5 | 99.5 | 99.5 |
| | (9.4 kP) fed | | | |
| 8 | 100 mg VBA fasted | 97.8 | 98.0 | 98.0 |
| 9 | 100 mg VBB fasted | 97.8 | 97.8 | 97.8 |

Results from VBE studies shown in Tables 12-14 show that 400 mg VBA and 400 mg VBB are anticipated to be bioequivalent to the Phase 3 pivotal clinical reference batch (400 mg Formulation 1) in the fasted state. Additionally, Tables 18-20 show that 400 mg VBA and 400 mg VBB also are anticipated to be bioequivalent to the Phase 3 pivotal clinical reference batch (400 mg Formulation 1) in the fed state.

Overall, the average GMR for 400 mg elacestrant dihydrochloride batches for Formulation 1 (pivotal clinical reference), VBA, and VBB show a reduction in the fasted and fed state, as expected from their dissolution characteristics, but keeping within BE limits for all the studies conducted. Therefore 400 mg elacestrant dihydrochloride batches for Formulation 1 (pivotal clinical reference), VBA, and VBB are anticipated to be bioequivalent to each other. See Table 22.

TABLE 22
Results of Estimated GMR Test/Reference for
400 mg Tablets in Fasted and Fed State

| | | Average from 10 VBE | | |
|---|---|---|---|---|
| | | Cₘₐₓ | AUC_{inf} | AUCₜ |
| VBE # | Clinical Simulation | Ratio | Ratio | Ratio |
| 4 | 400 mg Formulation 1, fasted | 103.3 | 103.4 | 103.4 |
| 5 | 400 mg VBA, fasted | 93.0 | 92.9 | 92.9 |
| 6 | 400 mg VBB, fasted | 92.8 | 93.0 | 93.0 |
| 10 | 400 mg Formulation 1, fasted | 98.6 | 99.2 | 99.2 |
| 11 | 400 mg VBA, fasted | 95.4 | 97.0 | 96.9 |
| 12 | 400 mg VBB, fasted | 89.6 | 89.0 | 89.0 |

### Example 8. Simulation of Repeat Dose Administration

Since the VBE results were obtained by comparing VBA and VBB performance for both strengths (100 mg and 400 mg) to the Phase 3 clinical references or BE study batches, simulations of repeat dose administration 200 mg OD and 400 mg OD were conducted in order to evaluate whether the minimal efficacious plasma concentration of 20 ng/mL (Cₘᵢₙ at steady state) could be reached. These doses correspond to the recommended doses for elacestrant dihydrochloride. These simulations were conducted in the fed state using 2×100 mg tablets of VBB (for 200 mg OD) and 400 mg VBB (for 400 mg OD). For comparison, the Phase 3 clinical reference batches were also simulated using the same dosing schedule.

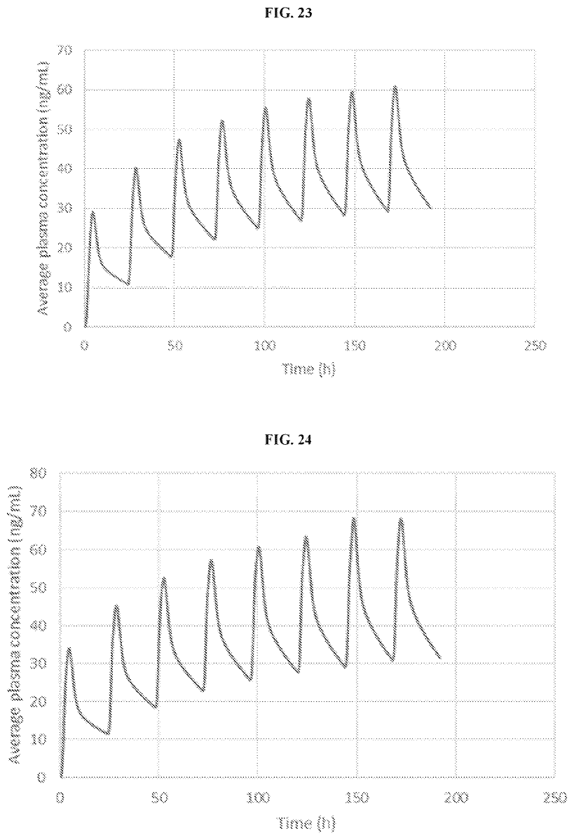

In the fed state with low-fat, low-calorie meal, the once daily administration of 2×100 mg elacestrant dihydrochloride batch VBB would lead to the average PK profile shown in FIG. 23. The calculated average Cₘᵢₙ at steady state is 38 ng/ml, with a geomean of 29 ng/ml, a low 90% CI of 18 ng/ml, and a high 90% CI of 41 ng/mL.

In the fed state with low-fat, low-calorie meal, the once daily administration of 2×100 mg elacestrant dihydrochloride batch Phase 3 clinical reference tablets (Formulation 1) would lead to the average PK profile shown in FIG. 24. The calculated average Cₘᵢₙ at steady state is 35 ng/ml, with a geomean of 31 ng/mL, a low 90% CI of 14 ng/mL, and a high 90% CI of 48 ng/mL.

Based on these findings, the steady state Cₘᵢₙ predicted after OD administration of 2×100 mg VBB tablets is very similar to that observed after administration of 2×100 mg Phase 3 clinical reference tablets (Formulation 1). Simulations performed with 2×100 mg Phase 3 clinical reference tablets (Formulation 1) in the fed state show that 60% of the patients would be expected to show C_{min,ss} above 20 ng/ml, and this value increases to 68% for VBB. Therefore, the efficacy of both Phase 3 clinical reference tablets (Formulation 1) and VBB 100 mg batches are anticipated to be the same at the 200 mg dose given OD.

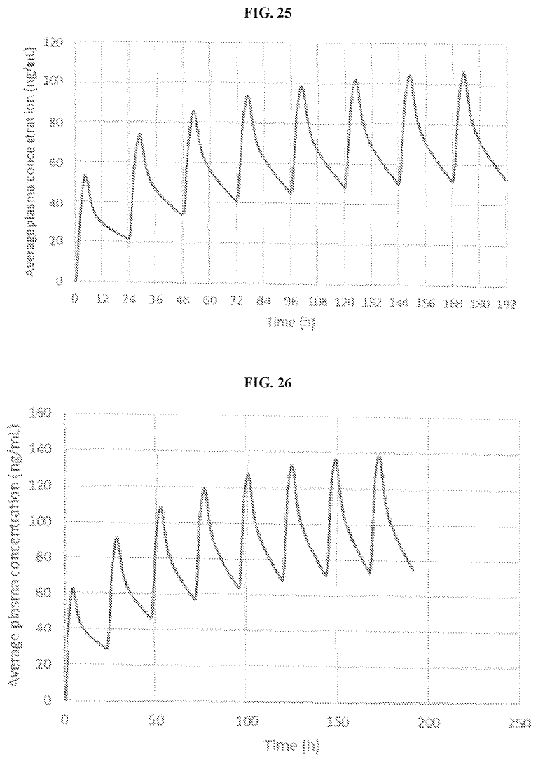

In the fed state with low-fat, low-calorie meal, the once daily administration of 400 mg elacestrant dihydrochloride batch VBB would lead to the average PK profile shown in FIG. 25. The calculated average Cₘᵢₙ at steady state is 38 ng/ml, with a geomean of 52 ng/ml, a low 90% CI of 23 ng/ml, and a high 90% CI of 81 ng/mL.

In the fed state with low-fat, low-calorie meal, the once daily administration of 400 mg elacestrant dihydrochloride tablets of Formulation 1 (Phase 3 clinical reference) would lead to the average PK profile shown in FIG. 26. The calculated average Cₘᵢₙ at steady state is 38 ng/ml, with a geomean of 73 ng/mL, a low 90% CI of 53 ng/mL, and a high 90% CI of 94 ng/mL.

As demonstrated by the simulated repeat dose administration, the model can predict the profiles observed for elacestrant dihydrochloride in the fasted and fed states following single or repeat administration. Late multiple peaks in the plasma profile of elacestrant can be observed from 6-10 hours post-administration. The role of enterohepatic recirculation can be ruled out because: (1) these multiple peaks are not observed on IV profiles; (2) no multiple peaks are observed up to 192 hour PK sampling despite numerous food administrations; and (3) for the 50 mg capsule administered in the fed state (not shown), where the dissolution and absorption is essentially complete in the upper GI tract (with 93% predicted absorption), no late peaks are observed. These late peaks can be explained by absorption from the cecum and colon, which could be due to: (1) faster dissolution of the dosage form from the cecum and colon due to the ability of the drug salt to create an acidic micro-environment; and/or (2) the effect of peristaltic forces on the terminal disintegration of the tablets after they have reached the lower GI tract, which could lead to faster drug release.

### Example 9. Bioavailability Simulations

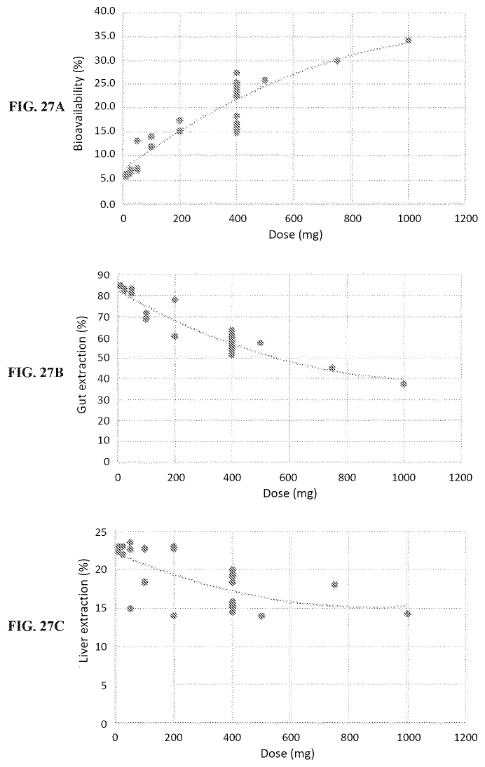

Without considering formulation or prandial status, the predicted bioavailability of elacestrant versus dose is shown in FIGS. 27A-C. The bioavailability estimated at 100 mg dose is around 10%, which is in agreement with measured values in the clinic. When comparing the effect of dose from 10 mg to 1000 mg on the drug bioavailability, the bioavailability is dose-dependent with more than dose proportionality in exposure. This is explained mechanistically in the model by a gradual saturation of the first pass gut extraction, which starts at 90% for low doses and falls to 40% for a 1000 mg dose (FIG. 27B).

### Example 10. Effect of Food

The effect of food was simulated at different doses, types of formulation, and types of food (e.g., low-calorie, low-fat meal; high-fat meal, etc.). The percent dissolved, absorbed, entering the portal vein, and in systemic circulation are overlaid with PK profiles (veinous return) for the various clinical scenarios where the same formulation was administered.

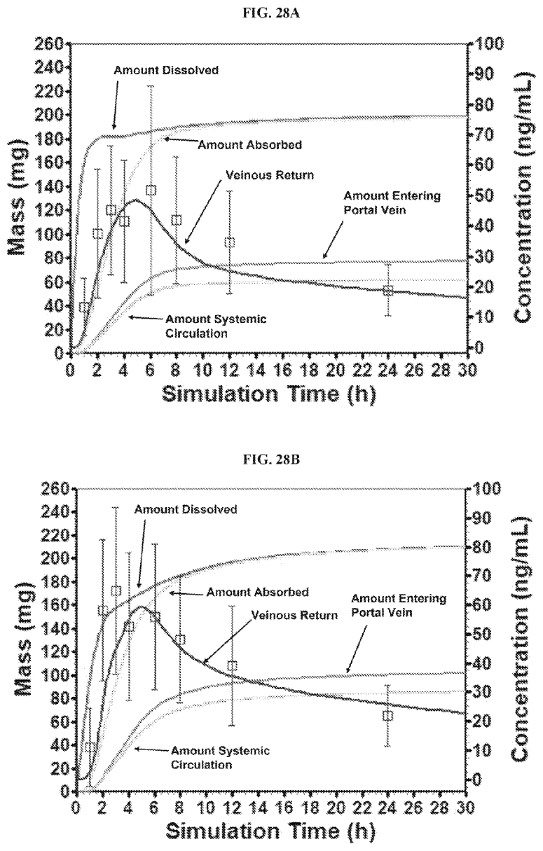

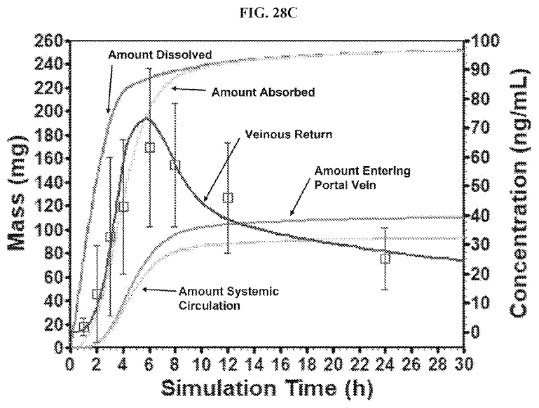

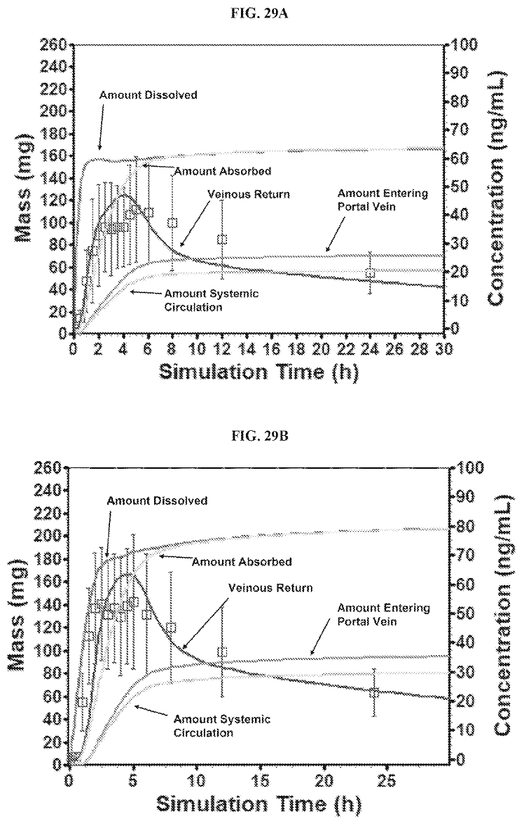

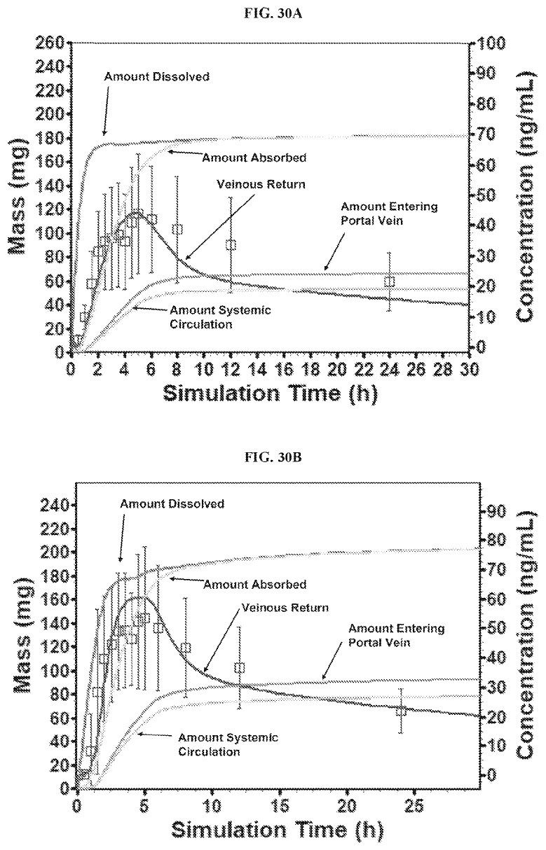

As shown in FIGS. 28A-C, FIGS. 29A-B, FIGS. 30A-B, and FIGS. 31A-B, the common observations for the effect of food on elacestrant dihydrochloride dissolution and absorption can be summarized as follows. Across all food effect studies, the fed state stomach leads to a slower dosage form dissolution compared to the fasted stomach, due to the pH difference between the fed and fasted stomach. Additionally, in the acidic stomach, solubility is higher, resulting in faster dissolution. However, in the fasted state, the gastric residence time is smaller, and after stomach emptying, the dissolution does not continue because the drug has reached a solubility limit and there is a low drug permeability in the intestine, which does not favor more dissolution or absorption. The combination of these two properties limits the drug fraction absorbed in the fasted state. Thus, in the fasted state the drug product dissolution rate in the stomach is not limiting the drug absorption but the amount dissolved prior to stomach emptying limits the extent of absorption.

In the fed state, the dissolution is slower, but because the solubility in the lumen of the small intestine is higher due to the presence of bile salts, the dissolution does not stop after stomach emptying. The drug dissolution also lasts longer in the stomach due to a higher residence time and volume.

These observations also underscore the differences between a high-fat, high-calorie meal (HH) and a low-fat, low-calorie meal (LL). For a low-fat, low-calorie meal, the stomach residence is less and the solubilization potential of the small intestinal fluids are less than for the high-fat, high-calorie meal. Therefore, for a low-fat low-calorie meal, almost the same amount of drug is dissolved at the end of stomach emptying at 400 mg dose for tablets in the fed state, compared to the fasted state (FIGS. 28A-B and FIGS. 29A-B). About 20 mg drug dose is dissolved during the intestinal transit.

High-fat, high-calorie meals lead to longer residence in the stomach, which increases the extent of dissolution from the stomach during digestion. For a 50 mg drug dose in a capsule, the dissolution is almost complete, followed by complete absorption, which leads to the largest food effect for elacestrant dihydrochloride, with an 87% increase in exposure. For a 400 mg tablet in study RAD1901-116, a high-fat high-calorie meal leads to the largest amount of drug dissolved at the end of stomach emptying. This results in drug supersaturation and precipitation, but the drug is able to redissolve during intestinal transit, owing to the effect of bile salts in the intestine. As a result, the effect of food on elacestrant dihydrochloride 400 mg tablets is less than that observed on 50 mg capsules (not shown).

### Example 11. Effect of Formulation on Bioavailability

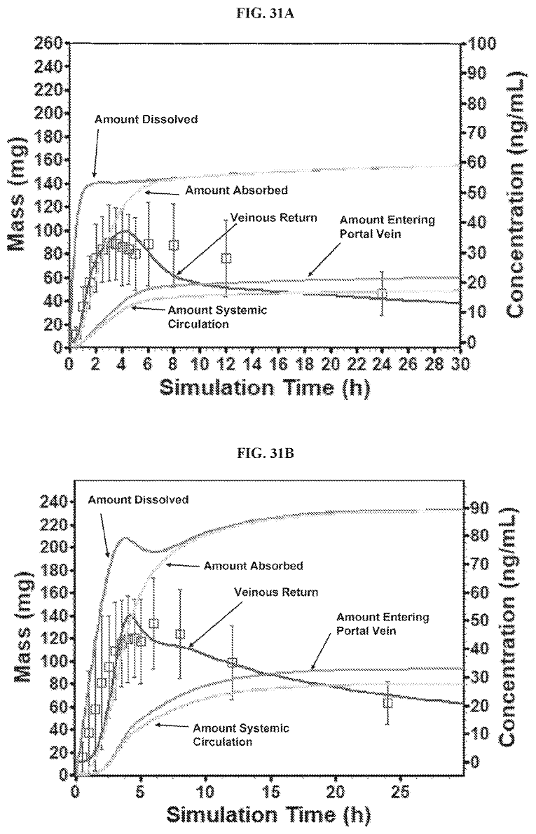

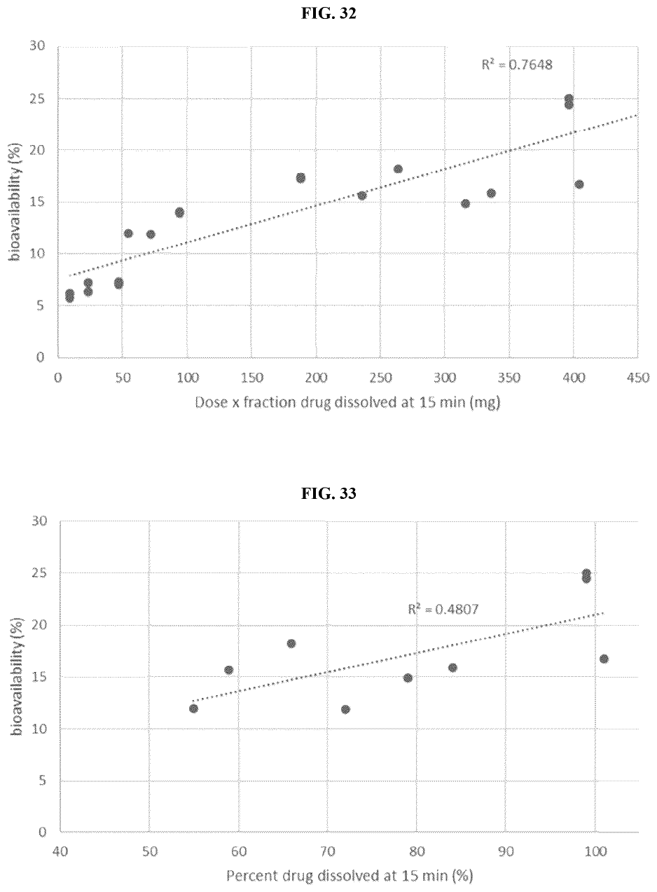

Referring to FIG. 32, the predicted bioavailability in the fasted state was plotted versus the product of drug dose and fraction of drug dissolved at 15 min in vitro across all formulations. The bioavailability of tablets was plotted against percent dissolved at 15 min, as shown in FIG. 33. As shown in the figures, the drug product dissolution influences the predicted drug bioavailability. As expected, the P-PSD determined from the dissolution batches tested in the clinic provides for a better descriptor of the in vivo dissolution. In addition, the acidification of the lower segments of the GI tract also depends on the drug concentration in these segments, which is also a function of the formulation. Thus, the correlations shown in FIG. 32 and FIG. 33 offer a simplified view of complex interplay of phenomena modeled in the PBBM. Nonetheless, the data demonstrates that QC dissolution methods are predictive of in vivo exposure.

### Example 12. Safe Space Establishment

Typically, safe space is built by comparing the upper and lower bounds of dissolution profiles such that all batches having dissolution profiles within these upper and lower limits of are bioequivalent. In addition, it is possible to define the safe space by comparing the upper and lower dissolution limits to the clinical/bioavailability batches or to an appropriate reference standard, including the pivotal batch, provided additional information (e.g., exposure-response data) is available. Two safe spaces for elacestrant dihydrochloride tablets were established, given that the proposed dissolution methods are strength dependent.

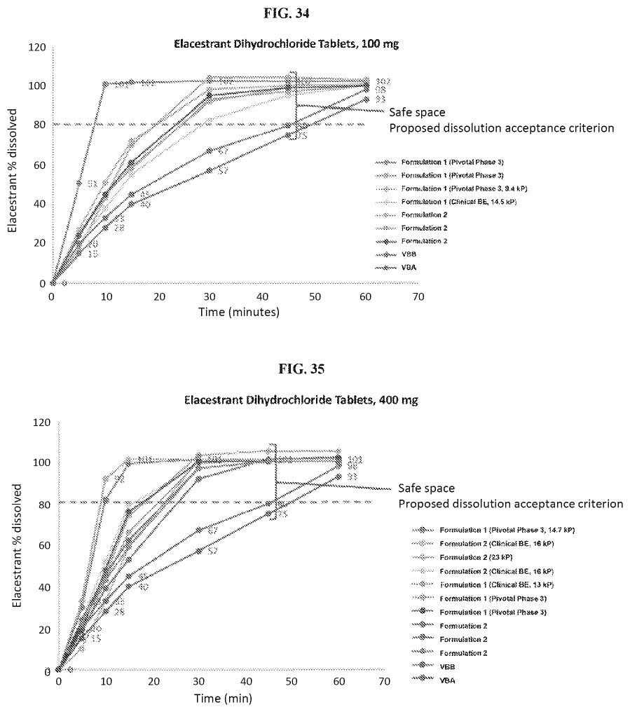

FIG. 34 illustrates the safe space for 100 mg elacestrant dihydrochloride tablets. The upper limit of the safe space for the 100 mg strength was established using the fastest dissolving batch, which was Formulation 1 tested in pivotal Phase 3 studies. As described in Example 6, using the validated PBBM, VBE studies showed that VBB 100 mg batch was bioequivalent to the Phase 3 clinical reference in the fasted and fed states and could define the lower bound of the safe space—that is, the dissolution specification (i.e., Q=75% at 45 min). Specifically, batches dissolving at a dissolution acceptance criterion of Q=80% at 45 min are expected to be bioequivalent to Phase 3 clinical reference for 100 mg strength.

Repeat dosing exposures in the fed state at the recommended effective (lower) dose with 200 mg (2×100 mg) VBB in the fed state are anticipated to lead to effective exposure in 68% of the population, which is similar to what was observed for Phase 3 clinical reference batch (60%) and similar to observations in the clinic, where approximately half the patients are below the threshold of 20 ng/ml at the dose of 200 mg. Further, it is anticipated that batches dissolving at the lower bound of this safe space would result in steady state plasma concentrations higher than those predicted for VBB, which led to equivalent exposure compared to the Phase 3 clinical reference, as discussed in Example 8.

Likewise for the 400 mg tablets, VBE studies showed that batch VBB 400 mg was bioequivalent to the pivotal Phase 3 clinical reference batch in the fasted and fed state. The limits of the safe space for the 400 mg strength were established using Formulation 2 (clinical BE, 16 kP) as the upper bound and batch VBB as the lower bound. (See FIG. 35.) As discussed in Example 7, using the validated PBBM, VBE studies showed that the VBB 400 mg batch was BE to the Phase 3 clinical reference in the fasted and fed state and could define the lower bound of the safe space-that is, the dissolution specification (i.e., Q=75% at 45 min). Specifically, batches dissolving at the acceptance criterion of Q=80% at 45 min in the QC dissolution method are then anticipated to be bioequivalent to the Phase 3 clinical reference batch for the 400 mg strength.

Further, as discussed in Example 8, repeat dosing exposures in the fed state at the recommended effective dose with 400 mg VBB are anticipated to lead to effective exposure in 90% of the population. In fact, the Cₘᵢₙ concentrations after repeating dosing of VBB were above the Cₘᵢₙ concentrations at steady state for batches tested in pivotal clinical trials supporting the efficacy of elacestrant dihydrochloride tablets.

Unless otherwise defined, all terms (including technical and scientific terms) used herein have the same meaning as commonly understood by one of ordinary skill in the art to which this invention belongs. The following references provide one of skill with a general definition of many of the terms used in this invention: Singleton et al., Dictionary of Microbiology and Molecular Biology (²ⁿd ed. 1994); The Cambridge Dictionary of Science and Technology (Walker ed., 1988); The Glossary of Genetics, ^{5t}h Ed., R. Rieger et al. (eds.), Springer Verlag (1991); and Hale & Marham, The Harper Collins Dictionary of Biology (1991); FDA Guidance for Industry: Bioequivalence Studies with Pharmacokinetic Endpoints for Drugs Submitted under an ANDA (2013). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 15%, preferably up to 10%, preferably up to 5%, and preferably up to 1% of a given value. Alternatively, the term can mean within an order of magnitude, preferably within 5-fold, and more preferably within 2-fold, of a value.

It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All patents, patent applications, provisional applications, and publications referred to or cited herein are incorporated by reference in their entirety, including all figures and tables, to the extent they are not inconsistent with the explicit teachings of this specification.

The compositions and methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

1. A solid pharmaceutical composition comprising:
a core comprising:
elacestrant dihydrochloride, present at a concentration of about 30 wt. % to about 60 wt. %, relative to the total weight of the core; and
silicified microcrystalline cellulose (SMCC) or a blend comprising microcrystalline cellulose and colloidal silicon dioxide, wherein the SMCC or the blend is present at a concentration of about 5 wt. % to about 25 wt. %, relative to the total weight of the core,
wherein the composition is a tablet having a hardness of at least about 5 kP as measured in accordance with USP <1217>.

2-8. (canceled)

9. The composition of claim 1, wherein the core further comprises microcrystalline cellulose, crospovidone, colloidal silicon dioxide, or magnesium stearate.

10. The composition of claim 1, wherein the SMCC or the blend is present at a concentration of about 15.0 wt. % to about 20 wt. %, relative to the total weight of the core.

11. The composition of claim 1, wherein the SMCC or the blend is present at a concentration of about 19.5 wt. %, relative to the total weight of the core.

12. The composition of claim 1, wherein the core comprises:
about 45.9 wt. % elacestrant dihydrochloride, relative to the total weight of the core;
about 26.4 wt. % microcrystalline cellulose, relative to the total weight of the core;
about 6.4 wt. % crospovidone, relative to the total weight of the core;
about 1.6 wt. % magnesium stearate, relative to the total weight of the core;
about 19.5 wt. % SMCC, relative to the total weight of the core; and
about 0.2 wt. % colloidal silicon dioxide, relative to the total weight of the core.

13. The composition of claim 1, further comprising a film coating the core.

14. The composition of claim 13, wherein the film is present at a concentration of about 1 wt. % to about 5 wt. %, relative to the total weight of the composition.

15. The composition of claim 1, wherein the elacestrant dihydrochloride is present in granules.

16. The composition of claim 15, wherein a first portion of the SMCC or the blend is intragranular and a second portion of the SMCC or the blend is extragranular.

17-20. (canceled)

21. A solid pharmaceutical composition comprising:
about 42 wt. % to about 47 wt. % elacestrant dihydrochloride;
about 20 wt. % to about 30 wt. % microcrystalline cellulose;
about 5 wt. % to about 10 wt. % crospovidone;
about 0.2 wt. % to about 3 wt. % magnesium stearate;
about 15 wt. % to about 25 wt. % SMCC; and
about 0.01 wt. % to about 1 wt. % colloidal silicon dioxide,
wherein the composition is a tablet.

22. The composition of claim 21, wherein the elacestrant dihydrochloride is present in granules.

23. The composition of claim 22, wherein a first portion of the SMCC is intragranular and a second portion of the SMCC is extragranular.

24-27. (canceled)

28. The composition of claim 1, wherein the elacestrant dihydrochloride is present in an amount of about 100 mg.

29. The composition of claim 28, wherein the tablet has a hardness of about 5.4 to about 16 kP, as measured in accordance with USP <1217>.

30. The composition of claim 28, wherein the tablet has a hardness of about 9 kP, as measured in accordance with USP <1217>.

31. The composition of claim 1, wherein the elacestrant dihydrochloride is present in an amount of about 400 mg.

32. The composition of claim 31, wherein the tablet has a hardness of about 12 to about 23 kP, as measured in accordance with USP <1217>.

33. The composition of claim 31, wherein the tablet has a hardness of about 17 kP, as measured in accordance with USP <1217>.

34. The composition of claim 1, wherein at least 85% of the elacestrant dihydrochloride is dissolved at 45 minutes in a medium of 0.01 N HCl, as measured in accordance with USP <711> and USP <1092> using apparatus 2, at a stirring speed of 75 rpm, a temperature of 37° C., in 500 mL or 1000 mL of the medium.

35-39. (canceled)

40. A method of manufacturing a solid pharmaceutical composition, the method comprising:
granulating an intragranular blend comprising elacestrant dihydrochloride and a first portion of a filler to provide an intragranular phase comprising granules;
mixing the intragranular phase with a second portion of the filler to provide a mixture; and
compressing the mixture at a pressure of about 3 kN to about 35 kN to produce a tablet,
wherein:
the granules comprise the elacestrant dihydrochloride and the first portion of the filler;
the second portion of the filler is extragranular;
the filler is SMCC or a blend comprising microcrystalline cellulose and colloidal silicon dioxide; and
the tablet has a hardness of about 5 kP to about 23 kP as measured in accordance with USP <1217>.

41-49. (canceled)

50. A method of manufacturing a solid pharmaceutical composition, the method comprising,
mixing elacestrant dihydrochloride, a first portion of SMCC, microcrystalline cellulose, and a first portion of crospovidone to provide an intragranular blend;
mixing the intragranular blend with a first portion of magnesium stearate to give a lubricated intragranular blend;
roller compacting the lubricated intragranular blend to provide an intragranular phase comprising granules;
mixing the intragranular phase with a second portion of SMCC, a second portion of crospovidone, and colloidal silicon dioxide to provide a compression blend;
mixing the compression blend with a second portion of magnesium stearate to provide the mixture;
compressing the mixture at a pressure of about 3 kN to about 35 kN to produce a tablet; and
coating the tablet with a film to give the composition,
wherein the granules comprise the elacestrant dihydrochloride, the first portion of SMCC, the microcrystalline cellulose, and the first portion of crospovidone, and
wherein the second portion of SMCC, the second portion of crospovidone, and the colloidal silicon dioxide are extragranular.

51-76. (canceled)

77. A method for treating breast cancer in a subject, the method comprising administering to the subject an effective amount of the composition according to claim 1.

78-89. (canceled)

90. A method for treating endometrial cancer in a subject, the method comprising administering to the subject an effective amount of the composition according to claim 1.

91-100. (canceled)

101. A method for treating breast cancer in a subject, the method comprising administering to the subject an effective amount of the composition according to claim 21.

102. A method for treating endometrial cancer in a subject, the method comprising administering to the subject an effective amount of the composition according to claim 21.
